(19) United States
(12) Patent Application Publication
(10) Pub. No.: US 2013/0121468 A1
(43) Pub. Date: May 16, 2013

(54) RADIOGRAPH IMAGING SYSTEM, RADIOGRAPH IMAGING METHOD, AND IMAGE DISPLAY METHOD

(71) Applicant: FUJIFILM Corporation, Tokyo (JP)

(72) Inventors: Yasunori OHTA, Kanagawa-ken (JP); Naoyuki NISHINO, Kanagawa-ken (JP)

(73) Assignee: FUJIFILM CORPORATION, Tokyo (JP)

(21) Appl. No.: 13/730,369

(22) Filed: Dec. 28, 2012

Related U.S. Application Data
(63) Continuation of application No. PCT/JP2011/065732, filed on Jul. 8, 2011.

(30) Foreign Application Priority Data
Jul. 30, 2010 (JP) 2010-172241
Jul. 30, 2010 (JP) 2010-172257
Jul. 30, 2010 (JP) 2010-172691

(51) Int. Cl. A61B 6/00

(52) U.S. Cl. CPC A61B 6/46
USPC 378/63

(57) ABSTRACT

In the disclosed radiograph imaging system, the transmission/reception of a signal is possible between on-site communication units and a waiting area communication unit. Also, the waiting area communication unit transmits at least an imaging menu to the on-site communication units.

### CROSS-REFERENCE TO RELATED APPLICATIONS AND PRIORITY CLAIMS

This application is a Continuation of International Application No. PCT/JP2011/065732 filed on Jul. 8, 2011, which was published under PCT Article 21(2) in Japanese, which is based upon and claims the benefit of priority from Japanese Patent Applications No. 2010-172241 filed on Jul. 30, 2010, No. 2010-172257 filed on Jul. 30, 2010, and No. 2010-172691 filed on Jul. 30, 2010, the contents all of which are incorporated herein by reference.

### TECHNICAL FIELD

The present invention relates to a radiographic image capturing system (radiograph imaging system) and a radiographic image capturing method (radiograph imaging method) for applying radiation from a radiation source to a subject, detecting radiation that has passed through the subject with a radiation detector, and converting the detected radiation into a radiographic image. The present invention also relates to an image displaying method (image display method) for displaying an image capturing menu concerning the radiographic image and capturing of the radiographic image.

### BACKGROUND ART

In the medical field, there have widely been used radiation image capturing apparatus, which apply radiation to a subject and guide radiation that has passed through the subject to a radiation conversion panel (radiation detector) in order to capture a radiographic image. Known forms of radiation conversion panels include a conventional radiographic film for recording a radiographic image by way of exposure, and a stimulable phosphor panel for storing radiation energy representing a radiographic image in a phosphor and emitting stimulated light representing the radiographic image if the stimulable phosphor panel is irradiated with stimulating light. The radiographic film with the recorded radiographic image is supplied to an image developing device, which develops the radiographic image. The stimulable phosphor panel is supplied to an image reading device, which reads the radiographic image from the stimulable phosphor panel as a visible image.

In an operating room or the like, it is necessary to read a recorded radiographic image immediately from a radiation conversion panel after the radiographic image has been captured for the purpose of quickly and appropriately treating the patient. As a radiation detector that meets such a requirement, there have been developed a radiation detector of a direct conversion type having a solid-state detector for converting radiation directly into an electric signal, and a radiation detector of an indirect conversion type having a scintillator for temporarily converting radiation into visible light and a solid-state detector for converting the visible light into an electric signal.

The radiation detector is housed in a radiation detecting cassette (cassette device), which is permeable to radiation.

As disclosed in Japanese Laid-Open Patent Publication No. 2003-093354, such radiographic image capturing apparatus are developed on the assumption that they will be used in order to capture radiographic images of patients in hospitals.

There are potential demands for capturing radiographic images outside of hospitals. To meet such demands, radiographic image capturing apparatus, which are mounted on motor vehicles dedicated for medical checkups, have been proposed in the art (see Japanese Laid-Open Patent Publication No. 2008-206740). However, such proposed radiographic image capturing apparatus mounted on medical checkup motor vehicles are relatively large in size. Needs have arisen for capturing radiographic images of persons suffering from natural disasters at disaster sites, or persons who are receiving home-care services in their homes. However, existing medical checkup motor vehicles cannot be used in the former applications as it is difficult to get them to disaster sites. Although existing medical checkup motor vehicles may be driven to homes of persons who are receiving home-care services, i.e., home care treatment sites, the image capturing process is highly burdensome to people to be imaged, because such people have to be taken from their homes into the medical checkup motor vehicle in order to capture radiographic images. Therefore, there have been demands for small-size portable radiographic image capturing apparatus for use at natural disaster sites or homes receiving home-care services.

There has been developed a portable radiographic image capturing apparatus, which can be folded into a compact form as a whole, as disclosed in Japanese Laid-Open Patent Publication No. 11-104117.

### SUMMARY OF INVENTION

### Problems to be Solved by the Invention

If a radiographic image capturing apparatus is reduced in overall size and weight, it becomes easy to carry around. A doctor or radiological technician carries the radiographic image capturing apparatus to a disaster site or a home care treatment site. At the disaster site or the home care treatment site, the doctor or radiological technician assembles the radiographic image capturing apparatus and captures a radiographic image using the same.

Radiographic images can usually only be captured by doctors or similarly qualified persons who are authorized as nationally licensed practitioners. In Japan, for example, persons who are legally permitted to engage in the business of applying radiation to a human body (to capture a radiographic image of the human body) are limited to doctors and dentists (hereinafter simply referred to as “doctors”) and medical radiological technicians (hereinafter simply referred to as “radiological technicians”) according to the Radiology Technicians Act. If a doctor or radiological technician who has legal authority concerning application of radiation to a subject is unable to go to a disaster site or a home care treatment site for some reason, then a person other than a doctor or radiological technician, i.e., a person who is not qualified as a medical radiological technician according to the Radiology Technicians Act (hereinafter referred to as “operator”), may take a radiographic image capturing apparatus to the site and perform a preparatory procedure to make the radiographic image capturing apparatus ready to capture radiographic images, e.g., to position a body region to be imaged of the subject with respect to a cassette device of the radiation detector. However, such an operator is not legally permitted to capture radiographic images of the subject with the radiographic image capturing apparatus. According to the present practice, a qualified person such as a doctor or radiological technician needs to go to the disaster site or the home care treatment site in order to capture radiographic images of the subject with the radiographic image capturing apparatus.

To eliminate the above shortcomings, the technologies disclosed in Japanese Laid-Open Patent Publication No. 2003-093354 and Japanese Laid-Open Patent Publication No. 2008-206740 may be applied in order to capture radiographic images of a subject, according to instructions from a doctor or a radiological technician who remains in a waiting location (e.g., a medical organization or a medical checkup motor vehicle) where the doctor or radiological technician cannot see the subject directly.

According to the technology disclosed in Japanese Laid-Open Patent Publication No. 2003-093354, an image (radiographic image) of an affected region of an emergency patient (subject) who has been carried into a medical organization is sent to the mobile terminal of a doctor who is not available at the medical organization, and the doctor is asked to give instructions as to a next radiographic image to be captured of the emergency patient. If the technology disclosed in Japanese Laid-Open Patent Publication No. 2003-093354 is applied directly, then the image of the affected region of the emergency patient, which is sent to the mobile terminal of the doctor in order to seek the doctor's instructions as to a next radiographic image to be captured, may possibly be a radiographic image that has been captured without the approval of the doctor. In addition, since the image of the affected region of the emergency patient needs to be sent to the mobile terminal of the doctor in order to seek the doctor's instructions as to a next radiographic image to be captured, the doctor is unable to instruct a person at the site how to capture a radiographic image of the patient in real time.

According to the technology disclosed in Japanese Laid-Open Patent Publication No. 2008-206740, the exposure of a subject to radiation is interrupted based on an optical image representing a body movement of the subject. Even if the technology disclosed in Japanese Laid-Open Patent Publication No. 2008-206740 were applied directly, the doctor is unable to instruct a person at the site to capture a radiographic image of the patient in real time.

In order for a doctor or radiological technician to send proper instructions as to how to capture radiographic images of a subject to a site while the doctor or radiological technician remains in a waiting location, it is desirable to capture an image of the site with a camera, to send an optical image (still image or moving image) captured by the camera to the waiting location, and to allow the doctor or radiological technician to give real-time instructions while viewing the optical image, which is displayed on a display screen of a display unit. At this time, it is desirable to display information, which the doctor or radiological technician wants to see, as largely as possible on the display screen, depending on how a radiographic image of the subject is to be captured, i.e., before and after a radiographic image of the subject is captured. However, the background art has not suggested an image displaying method for assisting a doctor or radiological technician in giving instructions as to how to capture radiographic images.

At sites, it may be assumed that the doctor or radiological technician needs to capture radiographic images of subjects, e.g., injured persons and disaster victims at disaster sites, which are not expected by the doctor or radiological technician. In this case, it is desirable for the doctor or radiological technician to be able to give real-time instructions as to how to capture radiographic images, to set (rewrite) an image capturing menu concerning capturing of radiographic images, and to capture radiographic images based on the set image capturing menu. However, no proposal has been presented in the background art with respect to setting of an image capturing menu depending on a subject at the site, and capturing of radiographic images based on the set image capturing menu.

In a case that radiographic images of several injured persons and disaster victims, i.e., subjects, at disaster sites, for example, are captured, if the captured radiographic images and information of the subjects are not accurately associated with each other, it may be extremely difficult for the doctor to interpret the radiographic images and make accurate diagnoses of subjects based on interpretation of the radiographic images. However, nothing is proposed in the background art concerning associating radiographic images and subject information with each other after radiographic images have been captured of several subjects.

### Objects of Invention

The present invention has been made in view of the above problems. It is an object of the present invention to provide a radiographic image capturing system, a radiographic image capturing method, and an image displaying method, which are capable of capturing an image of a subject without requiring a doctor or a radiological technician to travel directly to a disaster site or a home care treatment site.

Another object of the present invention is to provide a radiographic image capturing system and an image displaying method, which are capable of displaying information on a screen that a doctor or radiological technician wants to see in as a large scale as possible depending on a captured radiographic image, at a time that the doctor or radiological technician gives instructions to a disaster site or a home care treatment site while viewing an optical image of the site displayed on the screen.

Still another object of the present invention is to provide a radiographic image capturing system and a radiographic image capturing method, which are capable of acquiring an appropriate radiographic image of a subject based on an image capturing menu that has been set or rewritten depending on the subject at a disaster site or a home care treatment site.

Yet another object of the present invention is to provide a radiographic image capturing system and a radiographic image capturing method, which allow a doctor to perform an appropriate and efficient process of interpreting a radiographic image of a subject and to make a diagnosis of the subject by accurately associating information of the subject at a disaster site or a home care treatment site and the radiographic image of the subject with each other.

### Description of Invention

To achieve the above objects, there is provided in accordance with the present invention a radiographic image capturing system comprising:

a radiation source for outputting radiation;

a radiation detector for detecting radiation that has passed through a subject and converting the detected radiation into a radiographic image upon application of the radiation to the subject from the radiation source;

a portable terminal for controlling the radiation source and the radiation detector based on an image capturing menu concerning capturing of the radiographic image;

an on-site camera disposed together with the radiation source, the radiation detector, and the portable terminal at a site where the subject is present, for capturing an on-site image representative of the site;

an on-site communication unit disposed at the site;

a console disposed at a waiting location where a doctor or radiological technician having authority to apply the radiation to the subject waits while being unable to see the subject directly; and

a waiting location communication unit disposed at the waiting location,

wherein the on-site communication unit and the waiting location communication unit are able to send signals to and receive signals from each other; and

the waiting location communication unit transmits at least the image capturing menu to the on-site communication unit.

According to the present invention, since at least the image capturing menu is sent from the waiting location communication unit to the on-site communication unit, the portable terminal can control the radiation source and the radiation detector based on the image capturing menu received by the on-site communication unit in order to capture a radiographic image of the subject.

According to the present invention, furthermore, since signals can be sent and received between the on-site communication unit and the waiting location communication unit, an image of the site that is captured by the on-site camera and the radiographic image acquired by the radiation detector can be sent from the waiting location communication unit to the on-site communication unit.

According to the present invention, therefore, the doctor or radiological technician waiting at the waiting location while being unable to see the subject directly can give the operator at the site an instruction to capture a radiographic image of the subject in real time. Consequently, a radiographic image of the subject can be captured without the need for the doctor or radiological technician to travel directly to the site.

Specific features of the present invention (first through third inventions) will be described below in items [1] through [3].

[1] A radiographic image capturing system according to a first invention has the following arrangements in addition to those of the radiographic image capturing system described above according to the present invention.

In the radiographic image capturing system, the portable terminal includes a portable terminal display unit;

the console includes a console display unit;

the on-site communication unit transmits at least one of the radiographic image and the on-site optical image captured by the on-site camera to the waiting location communication unit; and

each of the portable terminal display unit and the console display unit is capable of displaying at least one of the on-site optical image, the radiographic image, and the image capturing menu, and of changing displayed content depending on how the radiographic image is captured.

An image displaying method according to the first invention comprises:

placing a radiation source, a radiation detector, a portable terminal, an on-site camera, and an on-site communication unit at a site where a subject is present, and placing a console and a waiting location communication unit at a waiting location where a doctor or radiological technician having authority to apply the radiation to the subject waits while being unable to see the subject directly;

capturing an on-site optical image representative of the site with the on-site camera, transmitting the on-site optical image from the on-site communication unit to the waiting location communication unit, and transmitting at least an image capturing menu from the waiting location communication unit to the on-site communication unit, for thereby allowing a portable terminal display unit of the portable terminal and a console display unit of the console to display at least one of the on-site optical image and the image capturing menu; and

controlling the radiation source and the radiation detector with the portable terminal based on the image capturing menu, so as to apply the radiation from the radiation source to the subject, and to convert the radiation that has passed through the subject into a radiographic image with the radiation detector, and transmitting the radiographic image from the on-site communication unit to the waiting location communication unit, for thereby allowing each of the portable terminal display unit and the console display unit to display at least one of the radiographic image, the on-site optical image, and the image capturing menu.

According to the first invention described above, before a radiographic image is captured, an on-site optical image is sent from the on-site communication unit to the waiting location communication unit, whereas an image capturing menu is sent from the waiting location communication unit to the on-site communication unit. Therefore, the console display unit and the portable terminal display unit display at least one of the image capturing menu and the on-site optical image.

The doctor or radiological technician waiting in the waiting location can give an appropriate instruction (e.g., to position the subject) to the operator at the site while viewing the on-site optical image displayed on the console display unit. If the image capturing menu is displayed on the console display unit, then by viewing the on-site optical image, the doctor or radiological technician can confirm whether or not the operator is working properly according to the content of the image capturing menu.

The operator can grasp what type of radiographic image of the subject is captured by viewing the image capturing menu displayed on the portable terminal display unit, and hence can perform an appropriate operation (e.g., to position the subject) depending on the image capturing menu. If the on-site optical image is displayed on the portable terminal display unit, then the operator can easily confirm the situation at the site.

After the radiographic image has been captured, the on-site optical image and the radiographic image are sent from the on-site communication unit to the waiting location communication unit, whereupon the console display unit and the portable terminal display unit display at least one of the image capturing menu, the on-site optical image, and the radiographic image.

Therefore, by viewing the radiographic image displayed on the console display unit, the doctor or radiological technician can judge whether or not an appropriate image according to the image capturing menu (an image suitable for the doctor to interpret and make a diagnosis of the subject) has been obtained. The operator can also know whether or not the radiographic image has been captured appropriately by viewing the radiographic image displayed on the portable terminal display unit.

According to the first invention, as with the present invention described above, therefore, the doctor or radiological technician waiting at the waiting location while being unable to see the subject directly can give the operator at the site an instruction to capture a radiographic image of the subject in real time while viewing the content displayed on the console display unit. Consequently, a radiographic image of the subject can be captured without the need for the doctor or radiological technician to travel directly to the site.

As described above, since signals representative of the image capturing menu, the on-site optical image, and the radiographic image are sent and received between the on-site communication unit and the waiting location communication unit, the console display unit is capable of displaying on the screen information that the doctor or radiological technician wants to see in as large a size as possible depending on how the radiographic image is captured. Further, the portable terminal display unit is capable of displaying on the screen information that the operator wants to see in as large a size as possible depending on how the radiographic image is captured.

[2] A radiographic image capturing system according to a second invention has the following arrangements in addition to those of the radiographic image capturing system described above according to the present invention.

In the radiographic image capturing system, the on-site communication unit transmits at least the on-site optical image captured by the on-site camera to the waiting location communication unit; and

the console is capable of changing content of the image capturing menu depending on a situation of the subject included in the on-site optical image before the radiographic image is captured.

A radiographic image capturing method according to the second invention comprises:

placing a radiation source, a radiation detector, a portable terminal, an on-site camera, and an on-site communication unit at a site where a subject is present, and placing a console and a waiting location communication unit at a waiting location where a doctor or radiological technician having authority to apply the radiation to the subject waits while being unable to directly see the subject;

capturing an on-site optical image representative of the site with the on-site camera, and transmitting the on-site optical image from the on-site communication unit to the waiting location communication unit;

changing content of an image capturing menu with the console depending on a situation of the subject included in the on-site optical image;

transmitting at least the image capturing menu from the waiting location communication unit to the on-site communication unit; and

controlling the radiation source and the radiation detector with the portable terminal based on the image capturing menu received through the on-site communication unit, so as to apply the radiation from the radiation source to the subject, and to detect the radiation that has passed through the subject and convert the detected radiation into a radiographic image with the radiation detector.

According to the second invention, before a radiographic image is captured, an on-site optical image is sent from the on-site communication unit to the waiting location communication unit. The console establishes (changes) content of the image capturing menu depending on a situation of the subject included in the on-site optical image, and sends the established image capturing menu from the waiting location communication unit to the on-site communication unit. As a result, the portable terminal controls the radiation source and the radiation detector based on the established image capturing menu in order to capture a radiographic image of the subject.

According to the second invention, as with the present invention described above, therefore, the doctor or radiological technician waiting at the waiting location while being unable to see the subject directly can give the operator at the site an instruction to capture a radiographic image of the subject in real time based on the on-site optical image received by the waiting location communication unit. Consequently, a radiographic image of the subject can be captured without the need for the doctor or radiological technician to travel directly to the site.

An appropriate radiographic image can be acquired by establishing (rewriting) an image capturing menu depending on the subject, and by capturing a radiographic image of the subject based on the established image capturing menu.

[3] A radiographic image capturing system according to a third invention has the following arrangements in addition to those of the radiographic image capturing system described above according to the present invention:

The radiographic image capturing system further comprises:

a memory;

wherein the on-site communication unit transmits at least one of the radiographic image and the on-site optical image captured by the on-site camera to the waiting location communication unit; and

the console or the portable terminal stores at least one of the image capturing menu, the radiographic image, and the on-site optical image in association with each other in the memory.

A radiographic image capturing method according to the third invention comprises:

placing a radiation source, a radiation detector, a portable terminal, an on-site camera, and an on-site communication unit at a site where a subject is present, and placing a console and a waiting location communication unit at a waiting location where a doctor or radiological technician having authority to apply the radiation to the subject waits while being unable to see the subject directly;

capturing an on-site optical image representative of the site with the on-site camera, transmitting the on-site optical image from the on-site communication unit to the waiting location communication unit, and transmitting at least an image capturing menu from the waiting location communication unit to the on-site communication unit;

controlling the radiation source and the radiation detector with the portable terminal based on the image capturing menu, so as to apply the radiation from the radiation source to the subject, and to convert the radiation that has passed through the subject into a radiographic image with the radiation detector, and transmitting the radiographic image from the on-site communication unit to the waiting location communication unit; and

storing at least the image capturing menu, the radiographic image, and the on-site optical image in association with each other in a memory, with the console or the portable terminal.

According to the third invention, an on-site optical image and a radiographic image are sent from the on-site communication unit to the waiting location communication unit, whereas at least an image capturing menu is sent from the waiting location communication unit to the on-site communication unit. The console or the portable terminal associates at least the image capturing menu, the radiographic image, and the on-site optical image with each other, and stores them in the memory.

According to the third invention, as with the present invention described above, therefore, the doctor or radiological technician waiting at the waiting location while being unable to see the subject directly can give the operator at the site an instruction to capture a radiographic image of the subject in real time, based on the on-site optical image received by the waiting location communication unit. Consequently, a radiographic image of the subject can be captured without the need for the doctor or radiological technician to travel directly to the site.

Since at least the image capturing menu, the radiographic image, and the on-site optical image are associated with each other and stored in the memory, information of the subject at the site and the radiographic image of the subject can be accurately associated with each other. As a result, the doctor is able to interpret the radiographic image and make a diagnosis of the subject appropriately and efficiently.

### BRIEF DESCRIPTION OF DRAWINGS

FIG. 1 is a perspective view of a radiographic image capturing system according to an exemplary embodiment of the present invention;

FIG. 2 is a perspective view of a radiographic image capturing apparatus shown in FIG. 1;

FIGS. 3A and 3B are perspective views of a radiation source device shown in FIG. 2;

FIG. 4 is a side elevational view of the radiographic image capturing apparatus shown in FIG. 2;

FIG. 5 is a side elevational view of the radiographic image capturing apparatus shown in FIG. 2;

FIG. 6 is a side elevational view of the radiographic image capturing apparatus shown in FIG. 2;

FIG. 7 is a perspective view showing the manner in which the radiographic image capturing apparatus shown in FIG. 2 is carried;

FIG. 8 is a view showing an internal structure of the radiation source device shown in FIG. 2;

FIG. 9 is a plan view of a cassette device shown in

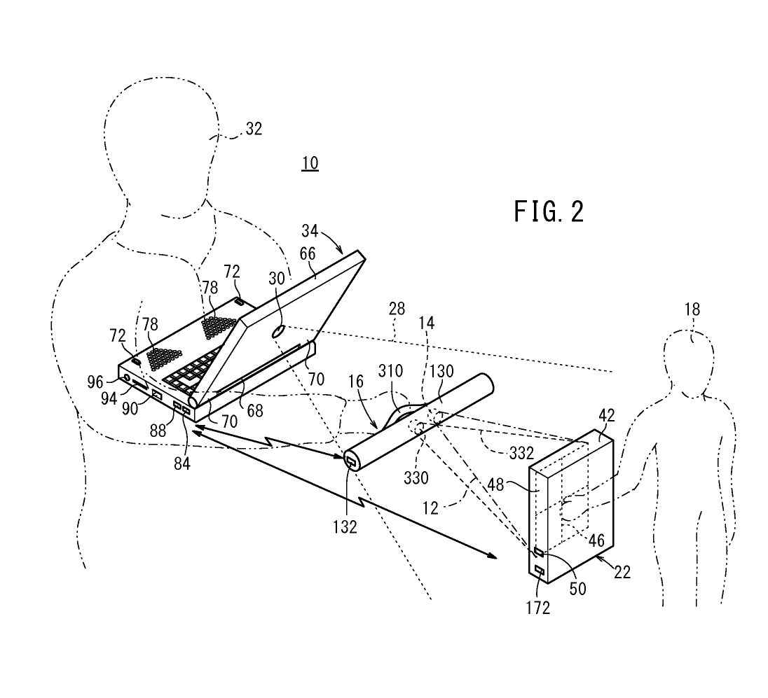

FIG. 2;

FIG. 10 is a view showing a matrix made up of pixels in a radiation detector;

FIG. 11 is a block diagram of the cassette device;

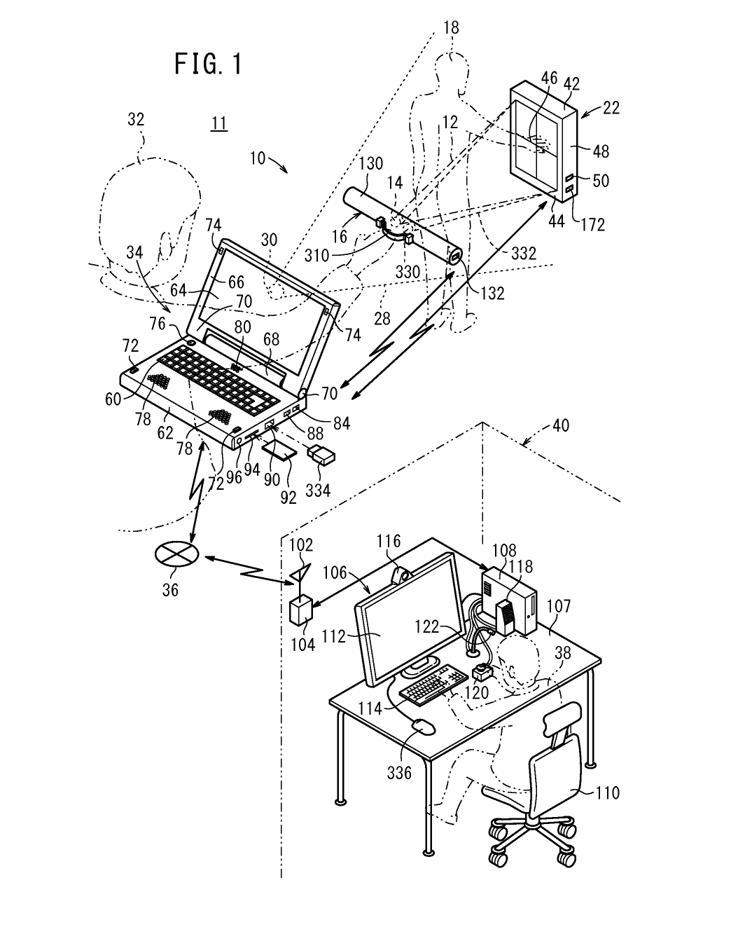

FIG. 12 is a block diagram of the radiographic image capturing apparatus shown in FIG. 1;

FIG. 13 is a block diagram of a medical organization shown in FIG. 1;

FIG. 14 is a flowchart of an operation sequence (first embodiment) of the radiographic image capturing system shown in FIG. 1;

FIG. 15 is a flowchart of the operation sequence of the radiographic image capturing system shown in FIG. 1;

FIG. 16 is a flowchart of the operation sequence of the radiographic image capturing system shown in FIG. 1;

FIG. 17 is a flowchart of the operation sequence of the radiographic image capturing system shown in FIG. 1;

FIG. 18 is a flowchart of the operation sequence of the radiographic image capturing system shown in FIG. 1;

FIG. 19 is a flowchart of the operation sequence of the radiographic image capturing system shown in FIG. 1;

FIG. 20 is a flowchart of the operation sequence of the radiographic image capturing system shown in FIG. 1;

FIG. 21 is a flowchart of another operation sequence (second embodiment) of the radiographic image capturing system shown in FIG. 1;

FIG. 22 is a flowchart of still another operation sequence (third embodiment) of the radiographic image capturing system shown in FIG. 1;

FIG. 23 is a view of a display screen displayed on a display unit of a console;

FIG. 24 is a view of a display screen on the display unit of the console;

FIG. 25 is a view showing at an enlarged scale a display area of the display screens shown in FIGS. 23 and 24;

FIG. 26 is a view showing at an enlarged scale a display area of the display screens shown in FIGS. 23 and 24;

FIG. 27 is a view showing respective display areas shown in FIGS. 23 and 24, which are displayed in two respective windows;

FIG. 28 is a view showing respective display areas shown in FIGS. 23 and 24, which are displayed in two respective windows;

FIG. 29 is a view showing respective display areas shown in FIGS. 23 and 24, which are displayed in two respective windows;

FIG. 30 is a view showing respective display areas shown in FIGS. 23 and 24, which are displayed in two respective windows;

FIG. 31 is a view of a display screen displayed on a display unit of a portable information terminal;

FIG. 32 is a view showing at an enlarged scale a display area of the display screen shown in FIG. 31;

FIG. 33 is a view showing at an enlarged scale a display area of the display screen shown in FIG. 31;

FIG. 34 is a view showing at an enlarged scale a display area of the display screen shown in FIG. 31;

FIG. 35 is a view showing respective display areas of the display screen shown in FIG. 31, which are displayed in three respective windows;

FIG. 36 is a view showing respective display areas of the display screen shown in FIG. 31, which are displayed in three respective windows;

FIG. 37 is a view showing respective display areas of the display screen shown in FIG. 31, which are displayed in three respective windows;

FIG. 38 is a view showing respective display areas of the display screen shown in FIG. 31, which are displayed in three respective windows;

FIG. 39 is a view showing respective display areas of the display screen shown in FIG. 31, which are displayed in three respective windows;

FIG. 40 is a view of a display screen, which is displayed after one of a plurality of procedures displayed in a list shown in FIG. 24 is selected;

FIG. 41 is a view showing a displayed list of image capturing conditions based on the selected procedure shown in FIG. 40;

FIG. 42 is a view of a display screen, which is displayed after one of the image capturing conditions displayed in the list shown in FIG. 41 is selected;

FIG. 43 is a view of a display screen, which is displayed immediately after order information, a procedure, and image capturing conditions are set;

FIG. 44 is a view of a display screen, which is displayed after order information, a procedure, and image capturing conditions are set;

FIG. 45 is a view of a display screen, which is displayed on the display unit of the console, for setting a thickness of a region to be imaged and an SID;

FIG. 46 is a view of a display screen, which is displayed on the display unit of the console, for setting a thickness of a region to be imaged and an SID;

FIG. 47 is a view of a display screen, which is displayed on the display unit of the console, for setting image capturing conditions;

FIG. 48 is a view of a display screen, which is displayed on the display unit of the console, for setting image capturing conditions;

FIG. 49 is a view of a display screen, which is displayed on the display unit of the console, for setting image capturing conditions;

FIG. 50 is a view of a display screen, which is displayed on the display unit of the console, for indicating that settings of an image capturing menu have been transmitted to a site;

FIG. 51 is a view of a display screen representing settings of an image capturing menu, which is displayed on a display unit of a portable information terminal;

FIG. 52 is a view of a display screen representing settings of an image capturing menu on the display unit of the portable information terminal;

FIG. 53 is a view of a display screen on the display unit of the portable information terminal, indicating that an acknowledgment of settings has been transmitted to the medical organization;

FIG. 54 is a view of a display screen on the display unit of the portable information terminal after the acknowledgment of settings has been transmitted;

FIG. 55 is a view of a display screen on the display unit of the console at a time that acknowledgment of settings has been received;

FIG. 56 is a view of a display screen asking the user to confirm settings;

FIG. 57 is a view of a display screen on the display unit of the console indicating a result of an authentication process;

FIG. 58 is a view of a display screen on the display unit of the console indicating a result of the authentication process;

FIG. 59 is a view of a display screen on the display unit of the portable information terminal indicating a result of the authentication process;

FIG. 60 is a view of a display screen on the display unit of the portable information terminal indicating a result of the authentication process;

FIG. 61 is a view of a display screen on the display unit of the portable information terminal at a time of positioning a subject;

FIG. 62 is a view of a display screen on the display unit of the console at a time of positioning a subject;

FIG. 63 is a view of a display screen on the display unit of the portable information terminal at a time of positioning a subject;

FIG. 64 is a view of a display screen on the display unit of the console at a time of positioning a subject;

FIG. 65 is a view of a display screen on the display unit of the portable information terminal at a time that positioning of the subject is OK;

FIG. 66 is a view of a display screen on the display unit of the portable information terminal at a time that positioning of the subject is OK;

FIG. 67 is a view of a display screen on the display unit of the portable information terminal indicating an instruction from a doctor or radiological technician;

FIG. 68 is a view of a display screen on the display unit of the portable information terminal indicating an instruction from the doctor or radiological technician;

FIG. 69 is a view of a display screen on the display unit of the portable information terminal at a time that positioning of the subject is OK;

FIG. 70 is a view of a display screen on the display unit of the portable information terminal indicating an instruction from the doctor or radiological technician;

FIG. 71 is a view of a display screen on the display unit of the console for judging whether or not positioning of the subject is OK;

FIG. 72 is a view of a display screen on the display unit of the console at a time of positioning the subject;

FIG. 73 is a view of a display screen on the display unit of the console at a time of positioning the subject;

FIG. 74 is a view of a display screen on the display unit of the console at a time that an exposure switch is displayed;

FIG. 75 is a view of a display screen on the display unit of the portable information terminal after a radiographic image has been captured;

FIG. 76 is a view of a display screen on the display unit of the portable information terminal after the radiographic image has been captured;

FIG. 77 is a view of a display screen on the display unit of the console for judging whether or not the radiographic image is OK;

FIG. 78 is a view of a display screen on the display unit of the console for indicating whether or not serial image capturing should be made implemented;

FIG. 79 is a view of a display screen on the display unit of the console indicating whether a radiographic image of the subject should be captured under different conditions, or whether a radiographic image of another subject should be captured;

FIG. 80 is a view of a display screen on the display unit of the portable information terminal indicating that capturing of a radiographic image is OK;

FIG. 81 is a view of a display screen on the display unit of the portable information terminal after a radiographic image has been captured;

FIG. 82 is a view of a display screen on the display unit of the console indicating whether or not the radiographic image is OK;

FIG. 83 is a view of a display screen on the display unit of the portable information terminal indicating that capturing of a radiographic image is No-Good and that the radiographic image needs to be recaptured;

FIG. 84 is a view of a display screen on the display unit of the portable information terminal at a time of positioning the subject in a case where a radiographic image of the subject needs to be recaptured;

FIG. 85 is a view of a display screen on the display unit of the console at a time of positioning the subject in a case where a radiographic image of the subject needs to be recaptured;

FIG. 86 is a view of a display screen on the display unit of the portable information terminal, showing at an enlarged scale a display area of a radiographic image;

FIG. 87 is a view of a display screen on the display unit of the console, showing at an enlarged scale a display area of a radiographic image;

FIG. 88 is a view of a display screen on the display unit of the console, showing, in four respective windows, a camera image of the subject, two camera images at a time of positioning the subject, and a radiographic image;

FIG. 89 is a view of a display screen on the display unit of the portable information terminal, showing, in four respective windows, a camera image of the subject, two camera images at a time of positioning the subject, and a radiographic image;

FIG. 90 is a view of a display screen on the display unit of the console, showing a camera image of the face of the subject including the subject's pupils;

FIG. 91 is a view of a display screen on the display unit of the portable information terminal, showing a camera image of the face of the subject including the subject's pupils;

FIG. 92 is a view of a display screen on the display unit of the console, showing a list of procedures;

FIG. 93 is a view of a display screen on the display unit of the console for setting order information;

FIG. 94 is a view of a display screen on the display unit of the console for judging whether or not settings of an image capturing menu are OK;

FIG. 95 is a view of a display screen on the display unit of the portable information terminal after a notification that settings are OK has been transmitted;

FIG. 96 is a view of a display screen on the display unit of the console at a time that notification that settings are OK is received;

FIG. 97 is a view of a display screen on the display unit of the portable information terminal after a notification that settings are No-Good has been transmitted;

FIG. 98 is a view of a display screen on the display unit of the console at a time that notification that the settings are No-Good is received;

FIG. 99 is a perspective view showing a manner in which the portable information terminal, the radiation source device, and the cassette device are charged in a medical organization;

FIG. 100 is a perspective view of a radiographic image capturing system (radiographic image capturing apparatus) according to a first modification;

FIG. 101 is a perspective view of a radiographic image capturing system (radiographic image capturing apparatus) according to a second modification;

FIG. 102 is a perspective view of a radiographic image capturing system (radiographic image capturing apparatus) according to a third modification;

FIG. 103 is a perspective view of a radiographic image capturing system (radiographic image capturing apparatus) according to a fourth modification;

FIG. 104 is a perspective view of a radiographic image capturing system (radiographic image capturing apparatus) according to a fifth modification;

FIG. 105 is a perspective view of a radiographic image capturing system (radiographic image capturing apparatus) according to a sixth modification;

FIG. 106 is a perspective view of a radiographic image capturing system (radiographic image capturing apparatus) according to a seventh modification;

FIG. 107 is a perspective view of a radiographic image capturing system (radiographic image capturing apparatus) according to an eighth modification;

FIG. 108 is a perspective view of a radiographic image capturing system (radiographic image capturing apparatus) according to a ninth modification;

FIG. 109 is a perspective view of a radiographic image capturing system (radiographic image capturing apparatus) according to a tenth modification;

FIG. 110A is a cross-sectional view showing schematically an internal structure of a cassette device according to an eleventh modification;

FIG. 110B is a cross-sectional view showing schematically and by way of example a scintillator shown in FIG. 110A; and

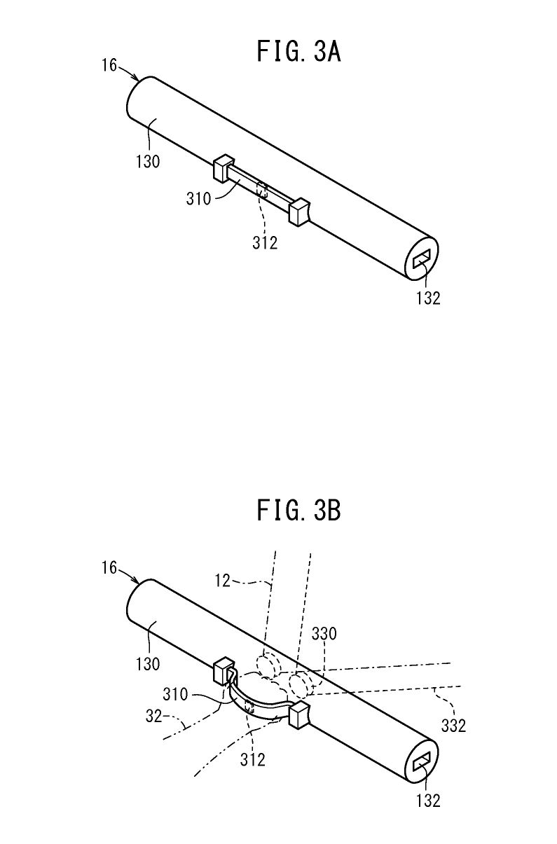

FIGS. 111A and 111B are perspective views showing another arrangement of the radiation source device shown in FIGS. 3A and 3B.

### DESCRIPTION OF EMBODIMENTS

A radiographic image capturing system according to a preferred exemplary embodiment of the present invention, in relation to a radiographic image capturing method and an image displaying method, will be described in detail below with reference to FIGS. 1 through 111B.

### [Arrangements of Embodiments]

As shown in FIGS. 1 and 2, a radiographic image capturing system 11 according to the present exemplary embodiment includes a radiographic image capturing apparatus 10. The radiographic image capturing apparatus 10 has a radiation source device 16, a cassette device 22, and a portable information terminal 34 (portable terminal).

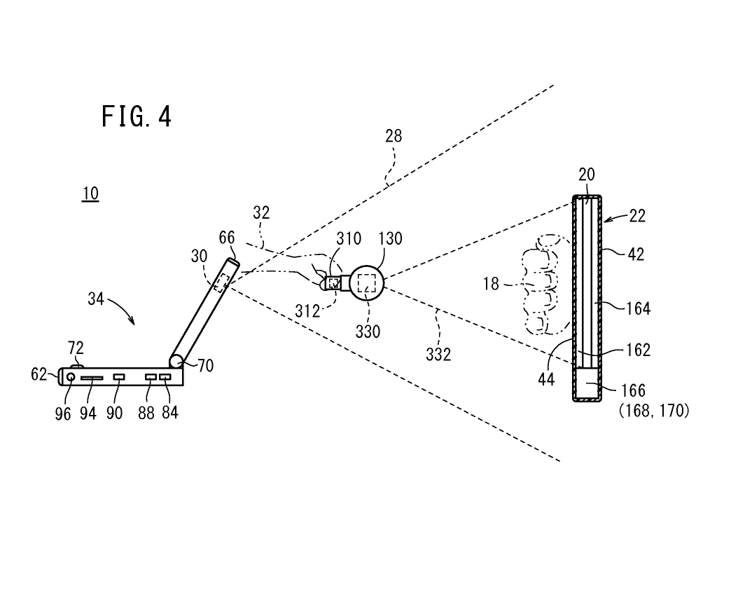

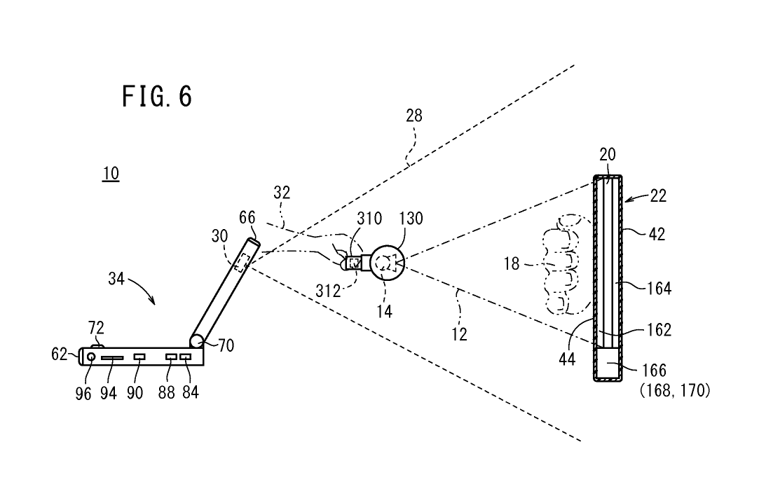

The radiation source device 16 houses therein a radiation source 14 for emitting radiation 12. The cassette device 22 houses therein a radiation detector 20 (see FIGS. 4 through 6) for converting radiation 12 that has passed through a subject 18 into a radiographic image. The portable information terminal 34, which is electrically connected through wireless links to the radiation source device 16 and the cassette device 22, can be operated by an operator 32 of the radiographic image capturing apparatus 10. The portable information terminal 34 is capable of sending signals to and receiving signals from a medical organization 40 (waiting location) to which a doctor (or radiological technician) 38 belongs, via a network 36 such as a public network or the like by way of wireless communications.

Radiographic images can usually be captured only by doctors or similarly qualified persons who are authorized as nationally licensed practitioners. The operator 32 refers to a person who is not qualified as a medical radiological technician according to the Radiology Technicians Act of Japan, or more specifically, a person other than doctors and dentists (hereinafter simply referred to as “doctors”) and medical radiological technicians (hereinafter simply referred to as “radiological technicians”) who have legal authority to apply radiation 12 to the subject 18.

According to the present exemplary embodiment, the subject 18 is present at a disaster site or a home care treatment site, whereas the doctor (or radiological technician) 38 is present (waits) in a remote medical organization 40 where the doctor 38 is unable to see the subject 18 directly. The doctor 38 is unable to go to the disaster site or the home care treatment site for certain reasons, whereas the operator 32 travels to the disaster site or the home care treatment site in lieu of the doctor 38.

According to the present exemplary embodiment, the radiographic image capturing apparatus 10 is brought to the above site for applying radiation 12 to the subject 18 in order to capture a radiographic image of the subject 18. The doctor 38 waits in a waiting location, which is a location where the doctor 38 is unable to see the subject 18 directly. Such a waiting location includes not only a disaster site or a home care treatment site, but also may be a location in the medical organization 40 where the doctor 38 is unable to see the subject 18 directly. Hereinafter, reference numeral 38 will be used to represent a doctor.

The portable information terminal 34 incorporates a web camera 30 (portable terminal camera, site camera) for capturing an image of a predetermined imaging area 28. The radiation source device 16 also incorporates a web camera 330 (radiation source camera, on-site camera) for capturing an image of a predetermined imaging area 332. The web camera 30 is integral with the portable information terminal 34, whereas the web camera 330 is integral with the radiation source device 16, which includes the radiation source 14.

The web camera 30 continuously captures an optical image of the imaging area 28, and outputs a camera image (moving image) which is representative of the continuously captured optical image. The web camera 30 can also intermittently capture optical images of the imaging area 28 at given time intervals, and output a camera image (still image) representative of the intermittently captured optical image or a camera image (still image) captured at a certain time.

The web camera 330, similar to the web camera 30, also continuously captures an optical image of the imaging area 332, and outputs a camera image (moving image) which is representative of the continuously captured optical image. The web camera 330 can also intermittently capture optical images of the imaging area 332 at given time intervals, and output a camera image (still image) representative of the intermittently captured optical image or a camera image (still image) captured at a certain time.

The web camera 30 is made integral with the portable information terminal 34 by being housed in the portable information terminal 34, as shown in FIGS. 1, 2, and 4 through 6. Alternatively, the web camera 30 may be made integral with the portable information terminal 34 by being coupled or connected to the portable information terminal 34, at least during times that the radiographic image capturing apparatus 10 is in use.

Therefore, the web camera 30 may be made integral with the portable information terminal 34 in any one of the following situations (1) through (3). (1) The web camera 30 is connected to the portable information terminal 34 by a cable, which is included in the radiographic image capturing apparatus 10. (2) The web camera 30 is connected to the portable information terminal 34 by a cable, which is provided by the operator 32. (3) During times that the radiographic image capturing apparatus 10 is in use, the portable information terminal 34 is coupled to the web camera 30, and during times that the radiographic image capturing apparatus 10 is serviced for maintenance or is not in use, the web camera 30 can be spaced or separated from the portable information terminal 34.

In order to enable the web camera 30 to be spaced from the portable information terminal 34 during times that the radiographic image capturing apparatus 10 is serviced for maintenance or is not in use, the web camera 30 may be coupled to the portable information terminal 34 by a coupling means such as a clip or the like. The web camera 30 is coupled to the portable information terminal 34 by the coupling means only at times that the radiographic image capturing apparatus 10 is in use. The coupling means may incorporate a ball joint to facilitate coupling of the web camera 30 to the portable information terminal 34 and to change the orientation thereof freely. If the web camera 30 is coupled to the portable information terminal 34 by way of such a coupling means, the web camera 30 and the portable information terminal 34 must be connected to each other through a wired link, e.g., a USB cable, or a wireless link.

If the web camera 30 and the portable information terminal 34 are connected to each other by a cable, then since the web camera 30 can independently be placed in a desired position within the range permitted by the length of the cable, the web camera 30 can be positioned with greater freedom than if the web camera 30 were incorporated in the portable information terminal 34.

The web camera 330 is made integral with the radiation source device 16 by being housed in the radiation source device 16, as shown in FIGS. 1, 2, 3B, and 4. Alternatively, the web camera 330 is made integral with the radiation source device 16 by being coupled or connected to the radiation source device 16, at least at times that the radiographic image capturing apparatus 10 is in use.

Therefore, the web camera 330 may be made integral with the radiation source device 16 in any of the following situations (1) through (3). (1) The web camera 330 is connected to the radiation source device 16 by a cable, which is included in the radiographic image capturing apparatus 10. (2) The web camera 330 is connected to the portable information terminal 34 by a cable, which is provided by the operator 32. (3) During times that the radiographic image capturing apparatus 10 is in use, the radiation source device 16 is coupled to the web camera 330, and at times that the radiographic image capturing apparatus 10 is serviced for maintenance or is not in use, the web camera 330 can be spaced or separated from the radiation source device 16.

In order to enable the web camera 330 to be spaced from the radiation source device 16 at times that the radiographic image capturing apparatus 10 is serviced for maintenance or is not in use, the web camera 330 may be coupled to the radiation source device 16 by a coupling means such as a clip or the like. The web camera 330 is coupled to the radiation source device 16 by the coupling means only at times that the radiographic image capturing apparatus 10 is in use. The coupling means may incorporate a ball joint to allow the web camera 330, which is coupled to the radiation source device 16, to freely change the orientation thereof. If the web camera 330 is coupled to the radiation source device 16 by the coupling means, the web camera 330 and the radiation source device 16 must be connected to each other through a wired link, e.g., a USB cable, or a wireless link.

If the web camera 330 and the radiation source device 16 are connected to each other through a cable, then since the web camera 30 can be placed independently in a desired position, the web camera 330 can be positioned with greater freedom than if the web camera 330 were incorporated in the radiation source device 16.

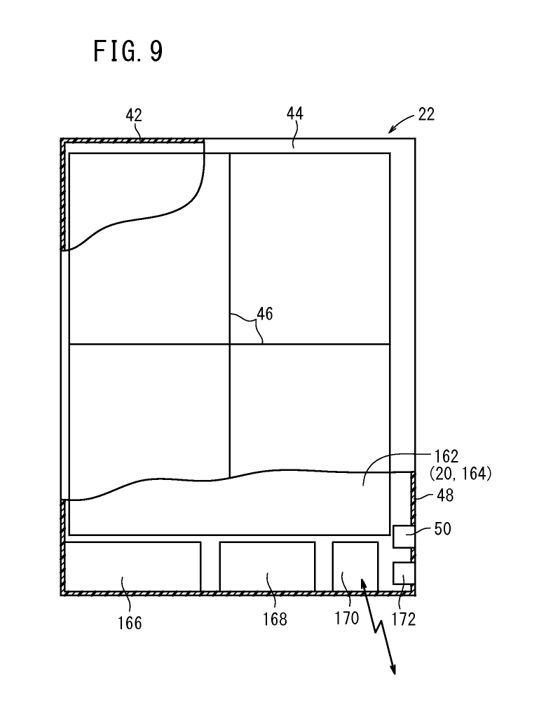

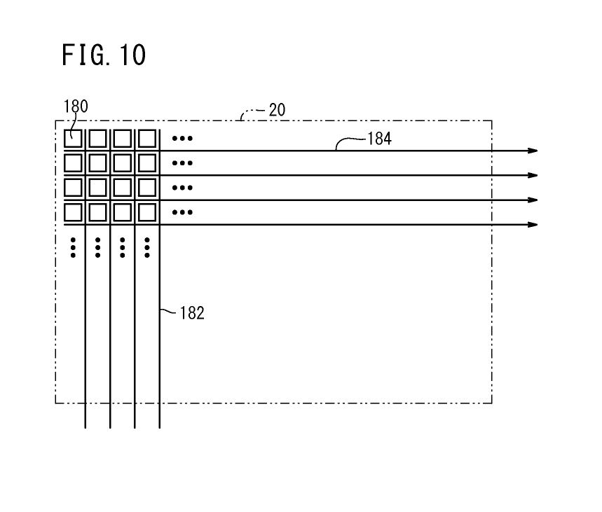

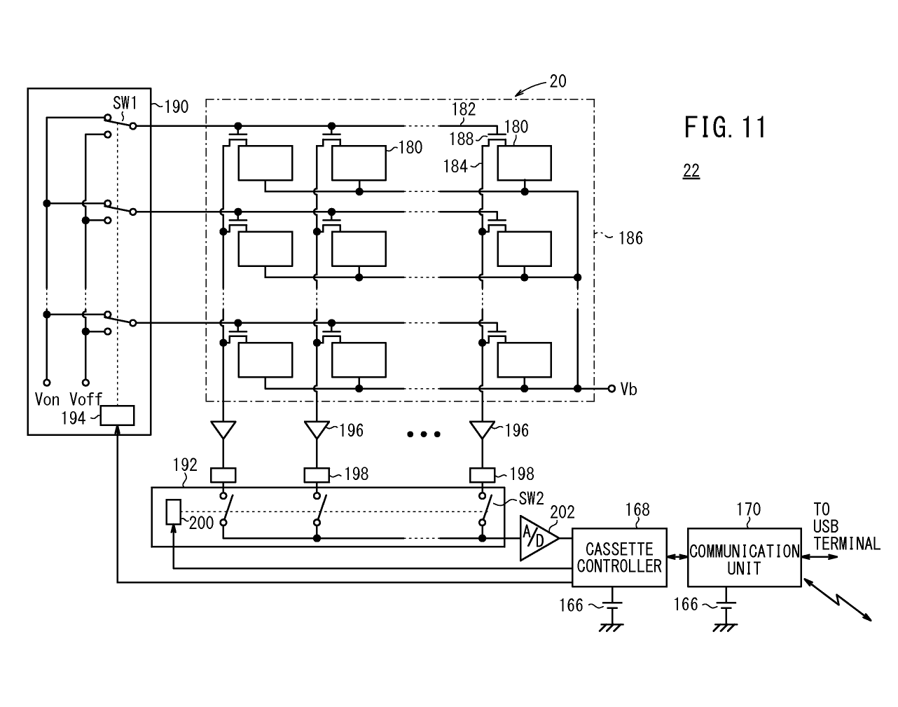

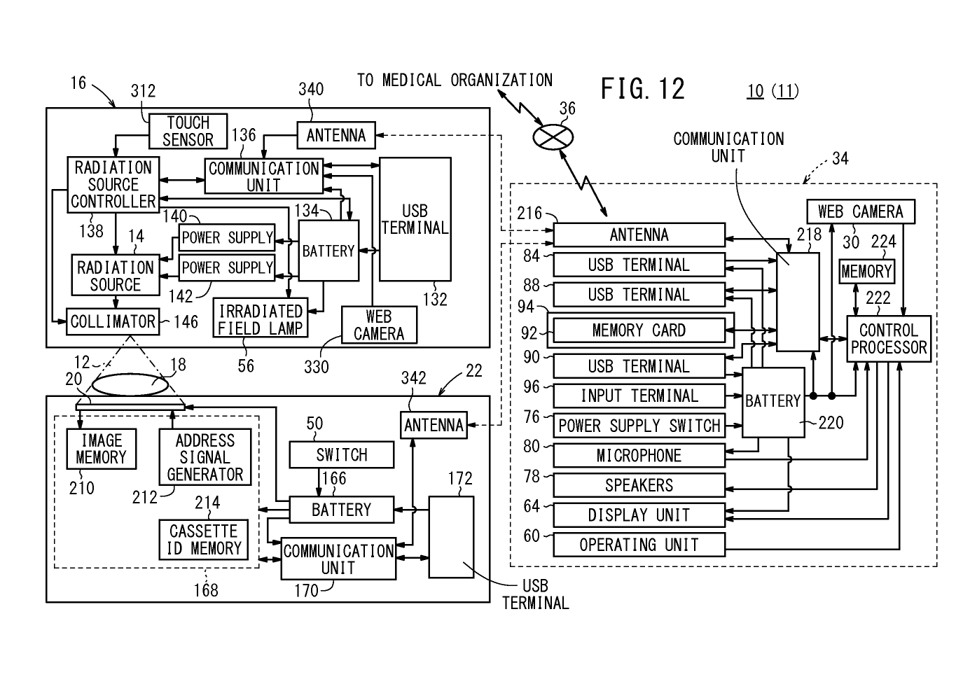

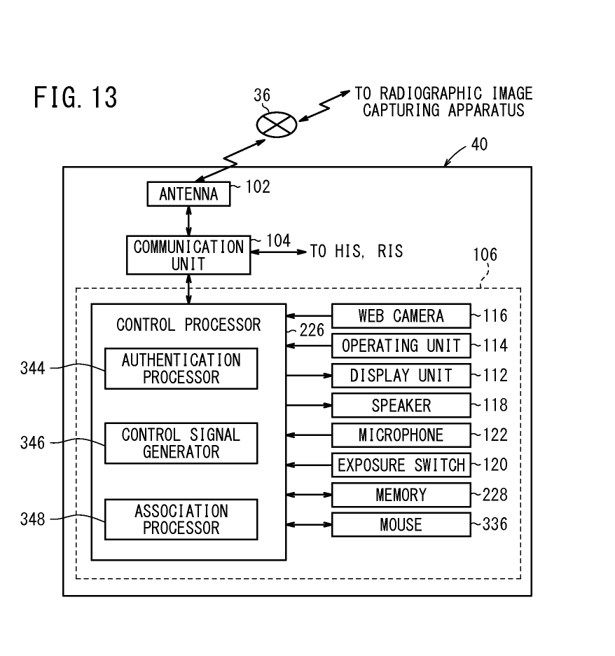

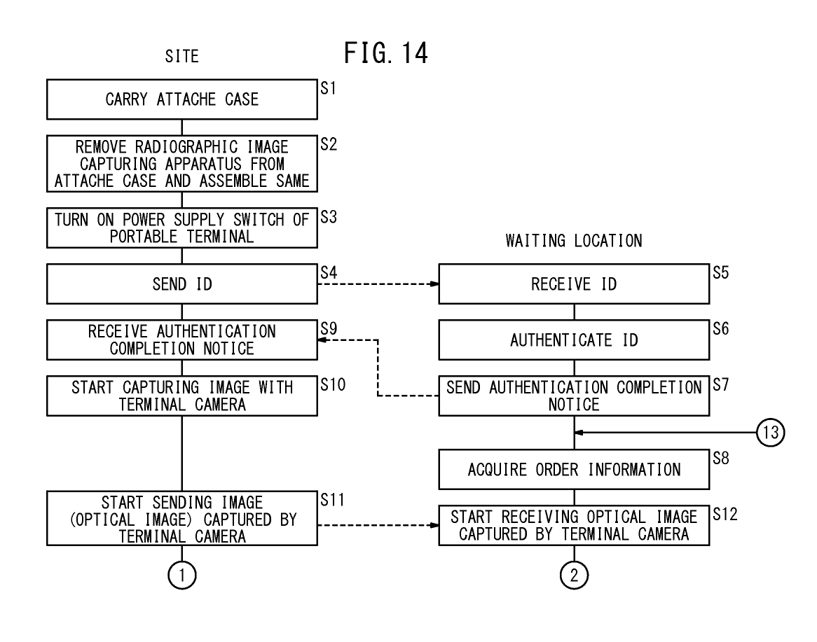

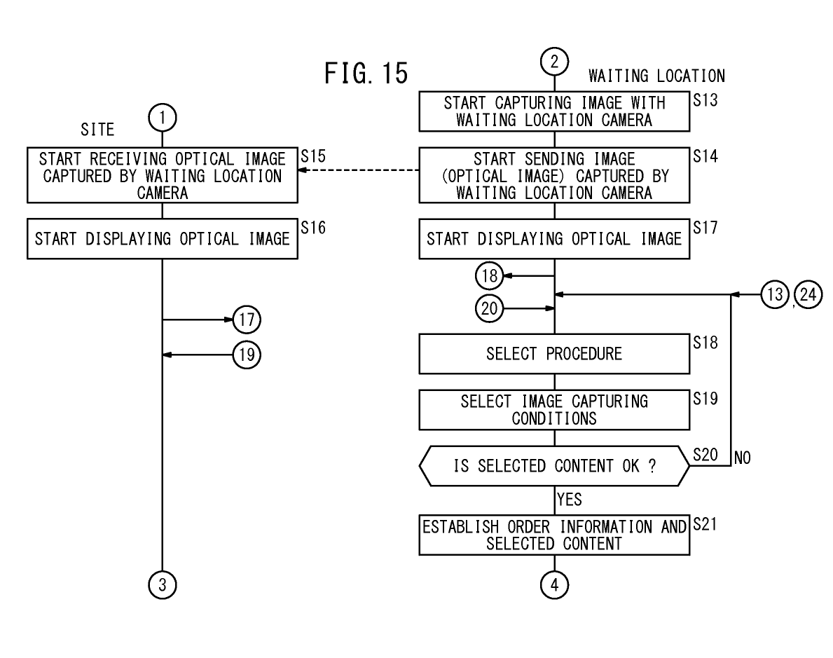

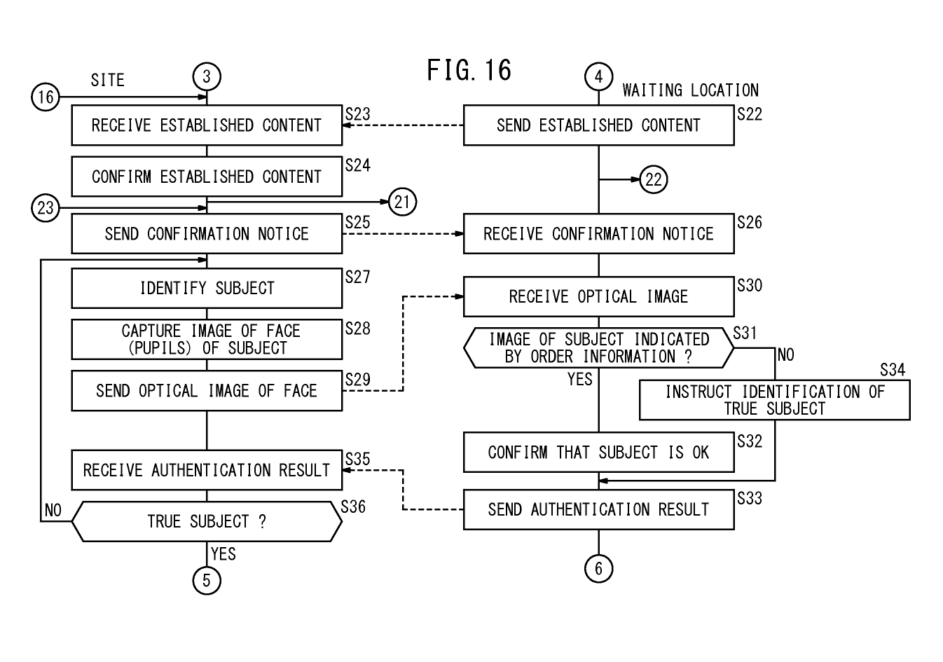

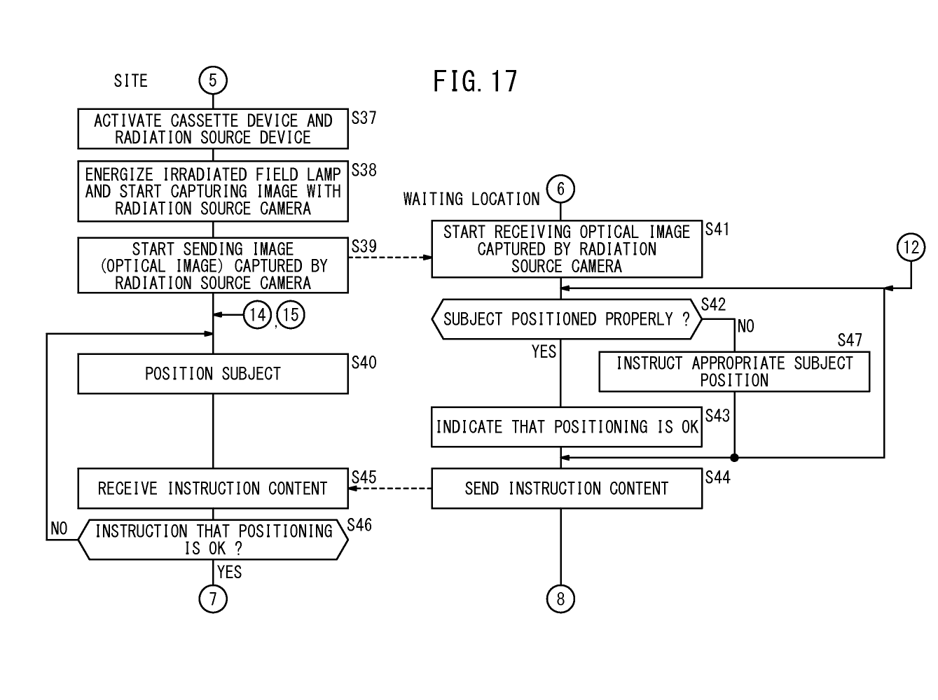

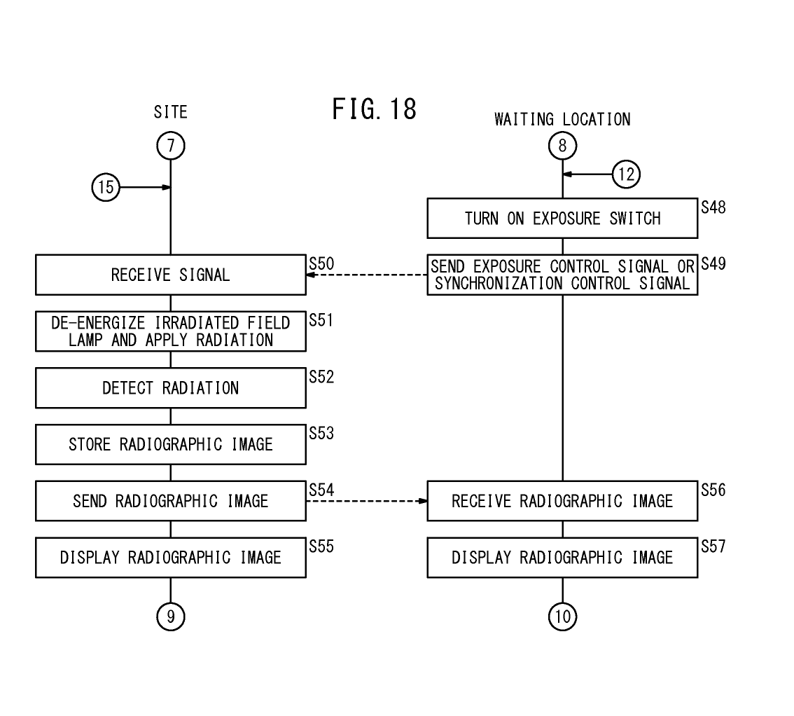

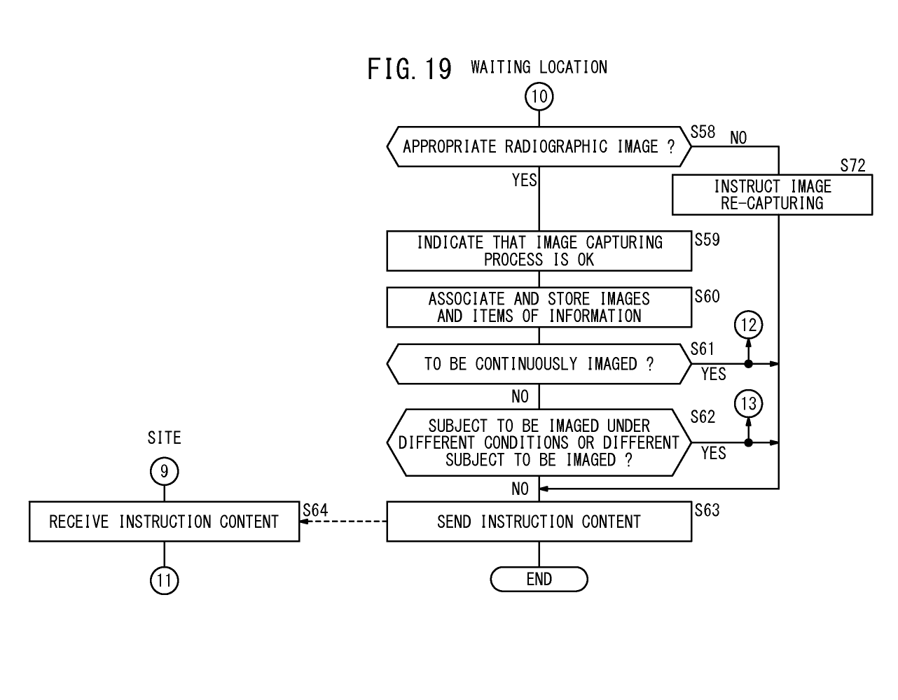

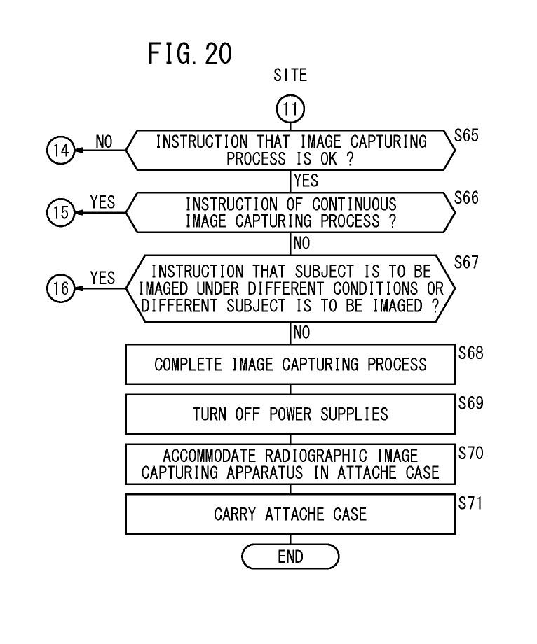

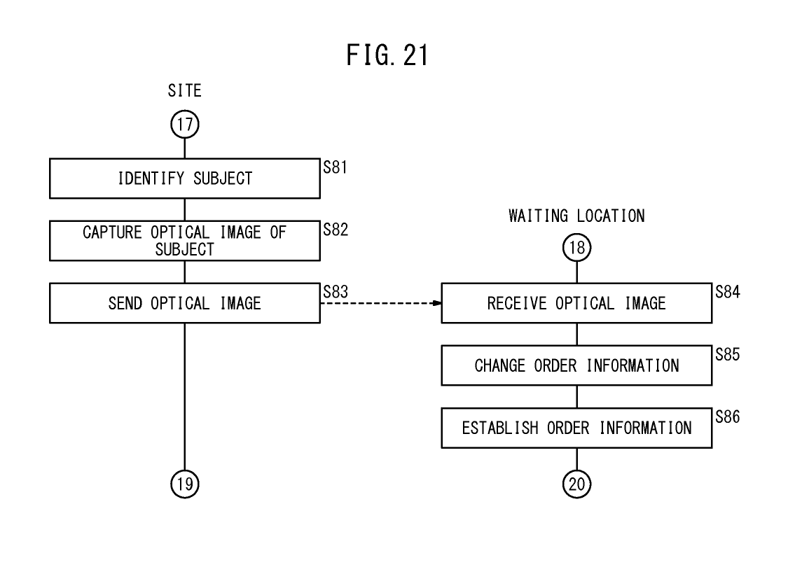

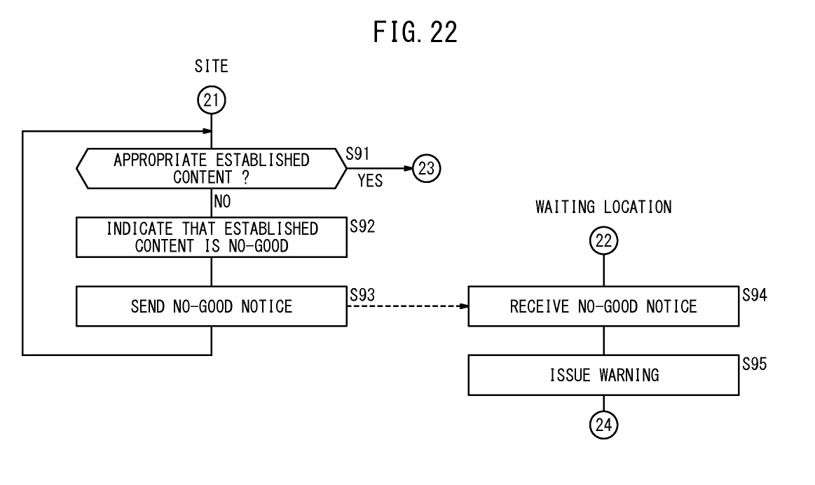

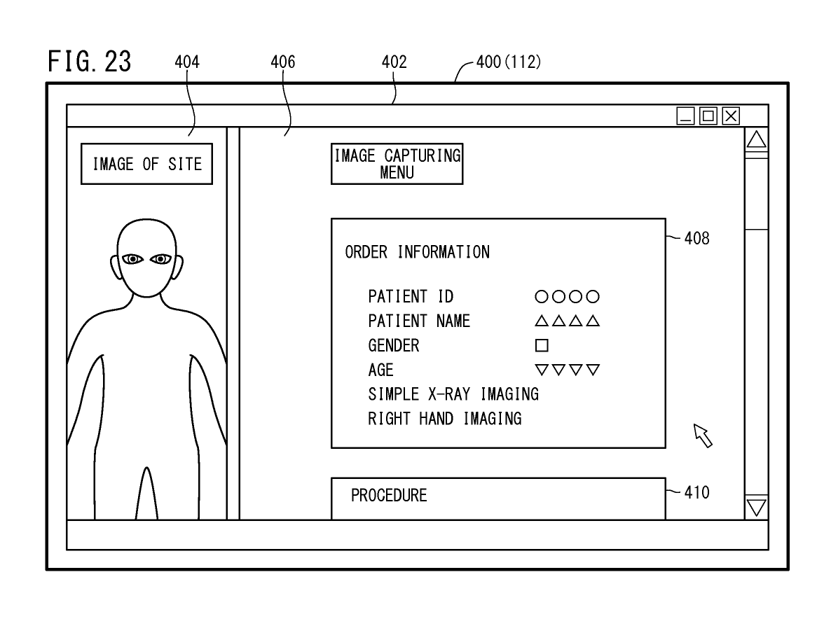

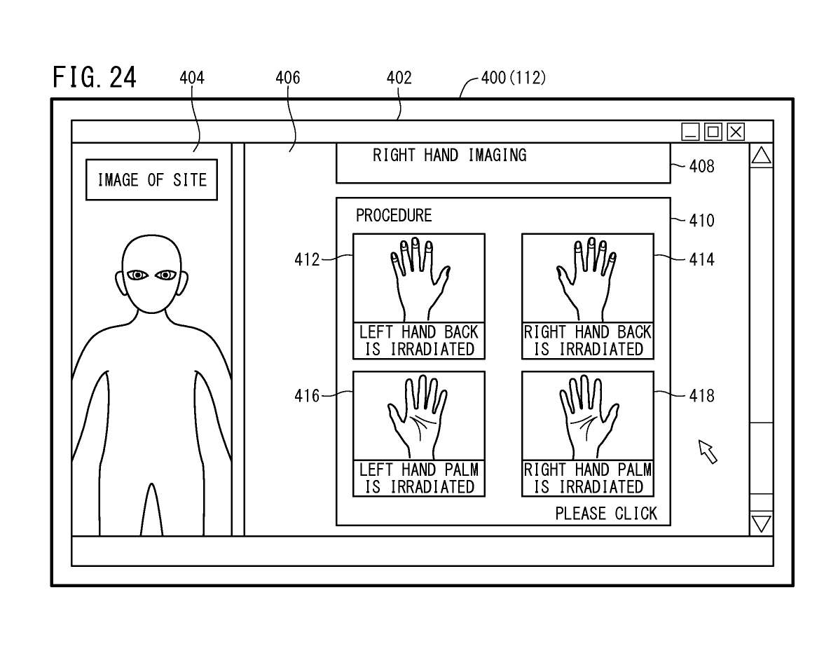

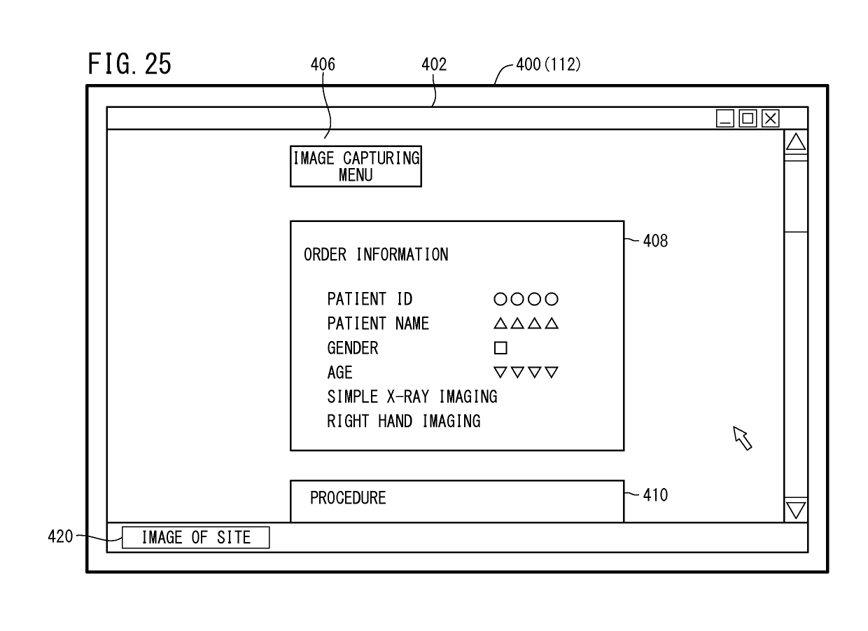

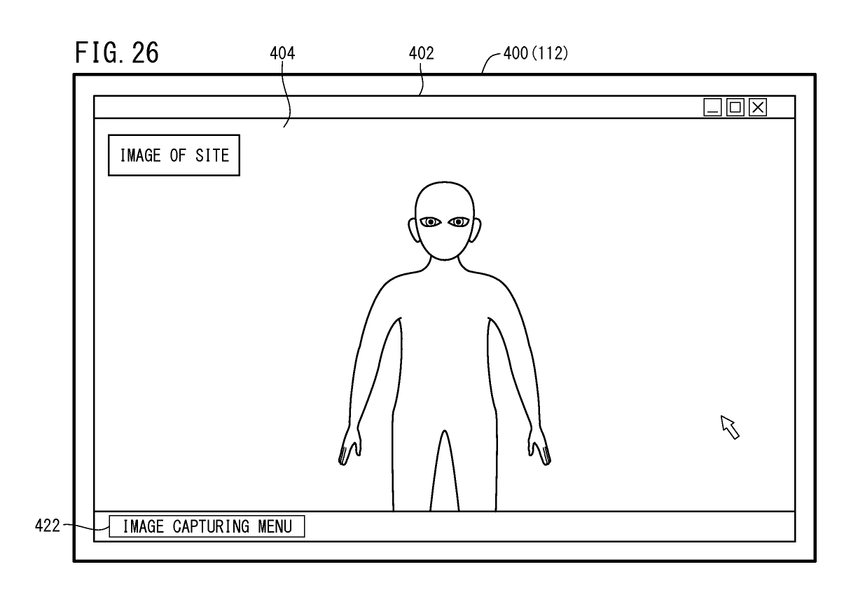

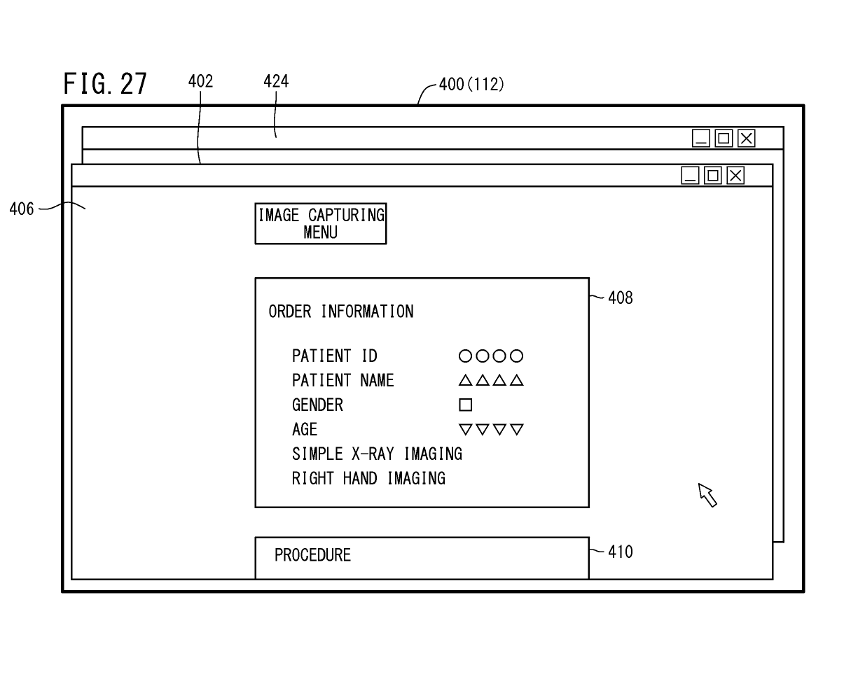

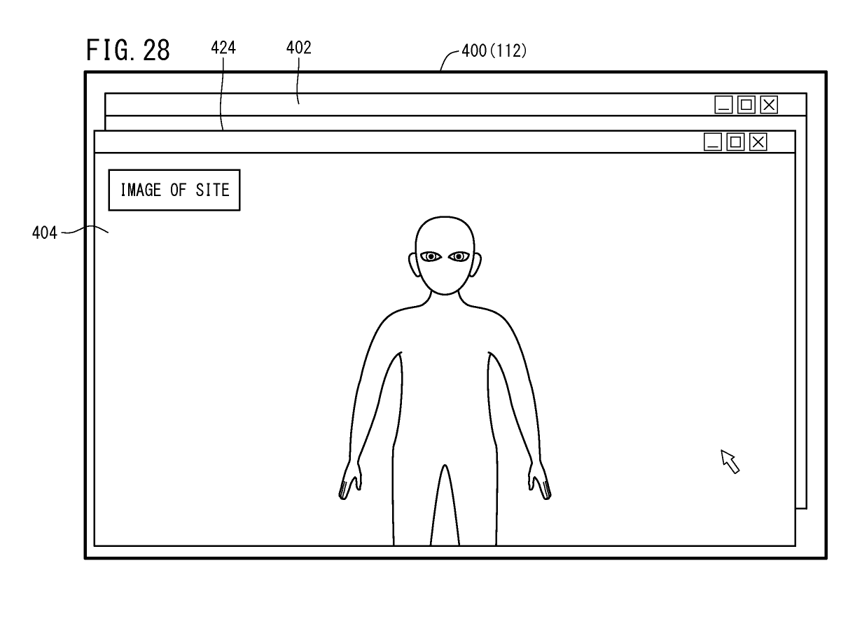

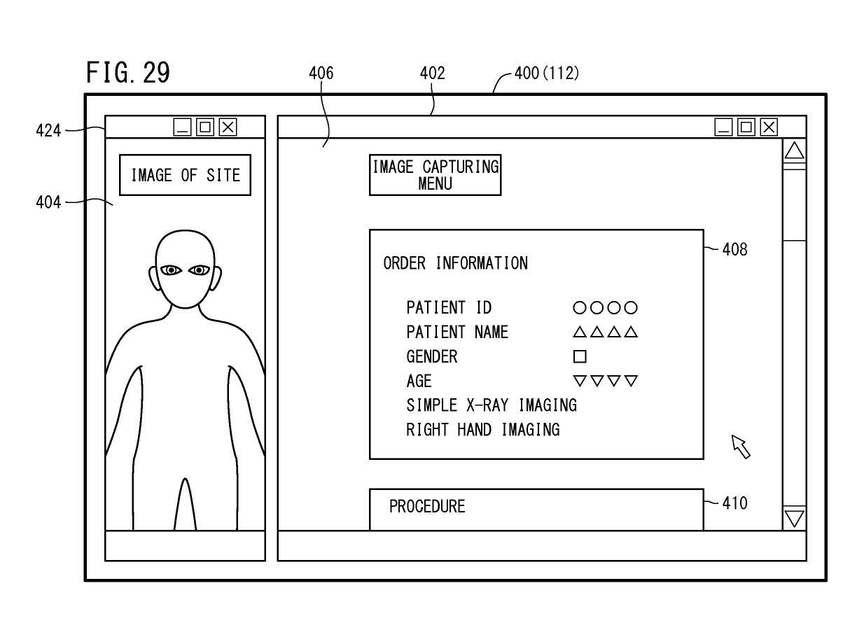

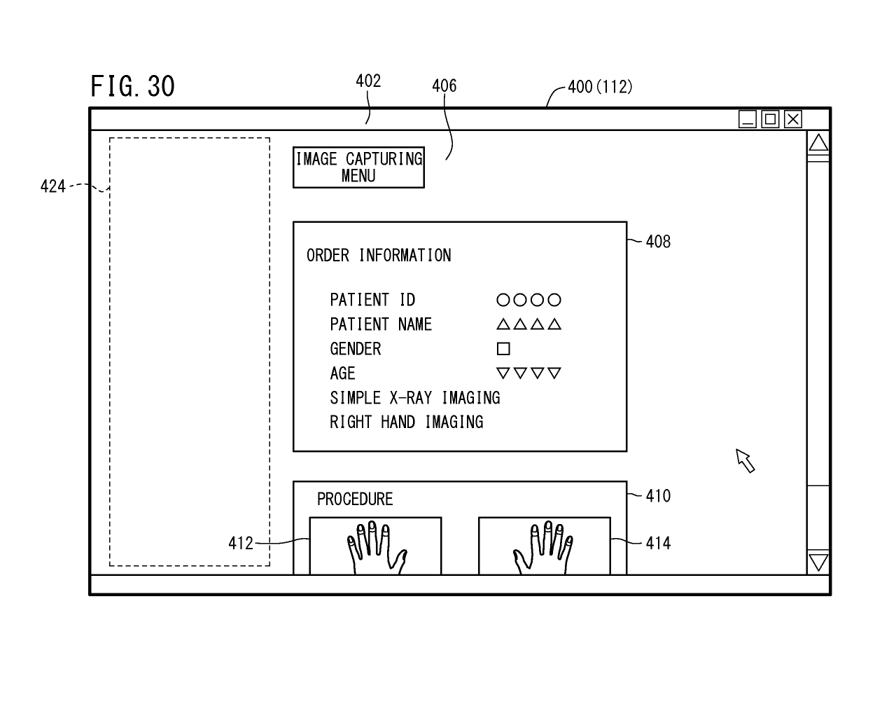

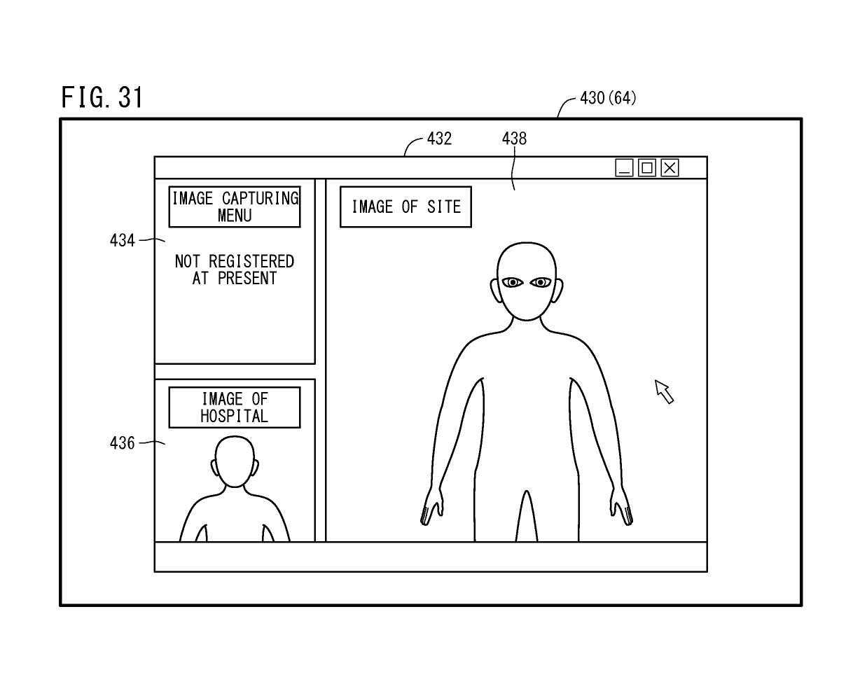

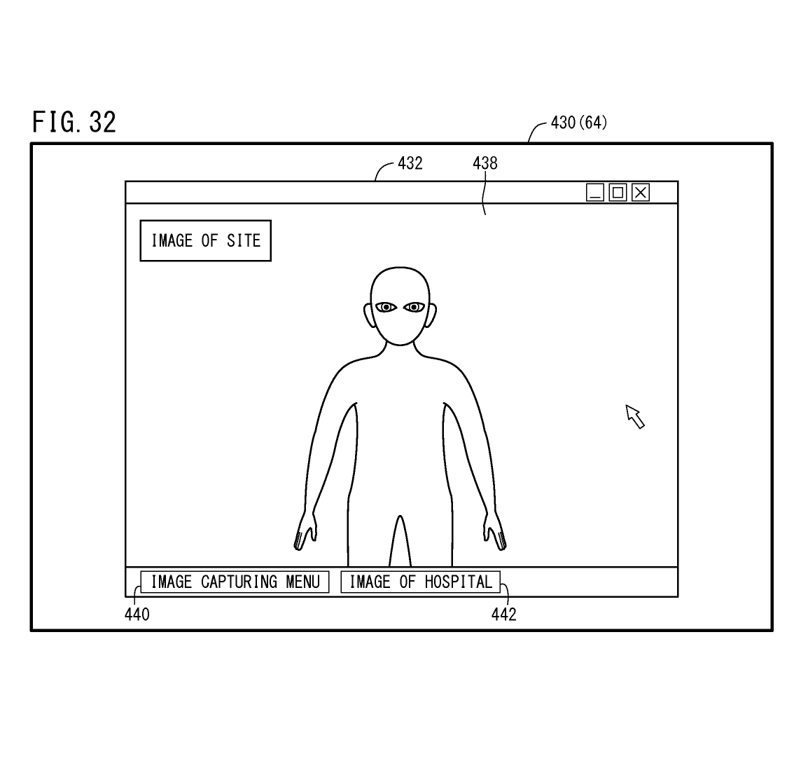

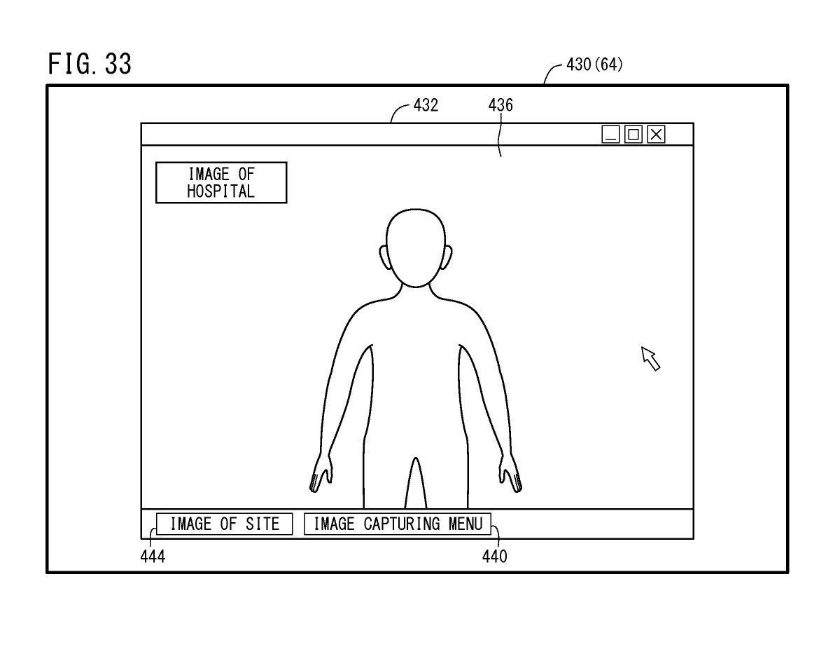

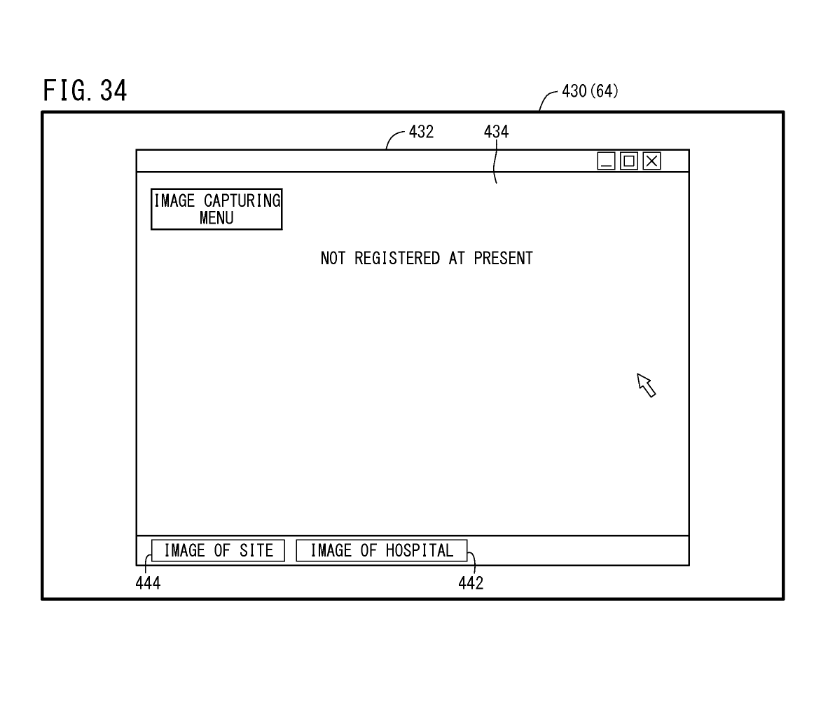

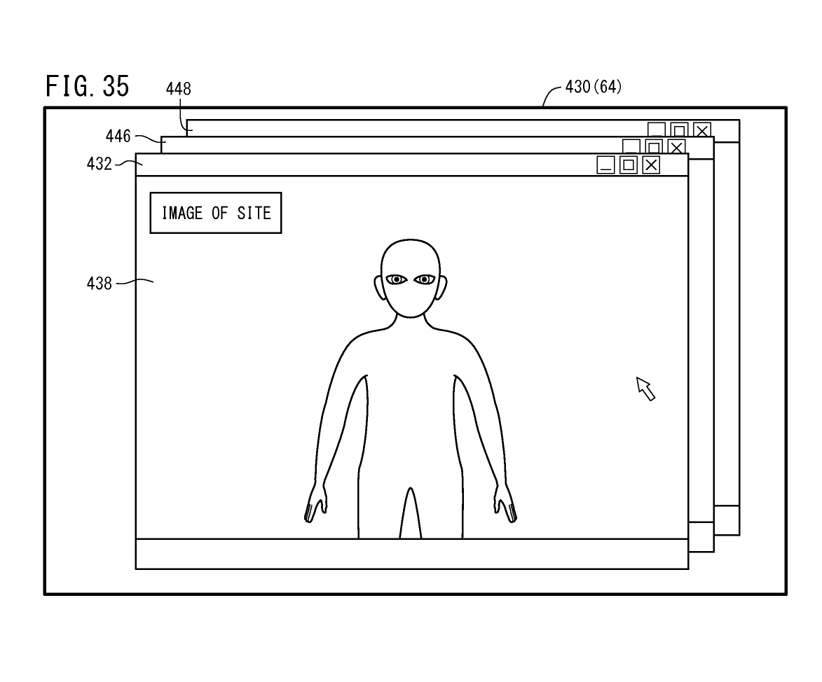

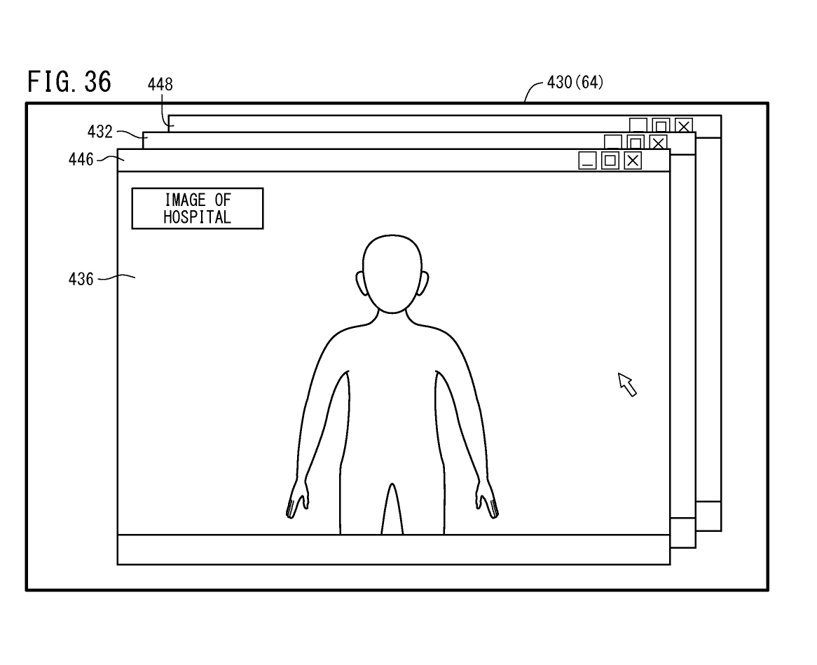

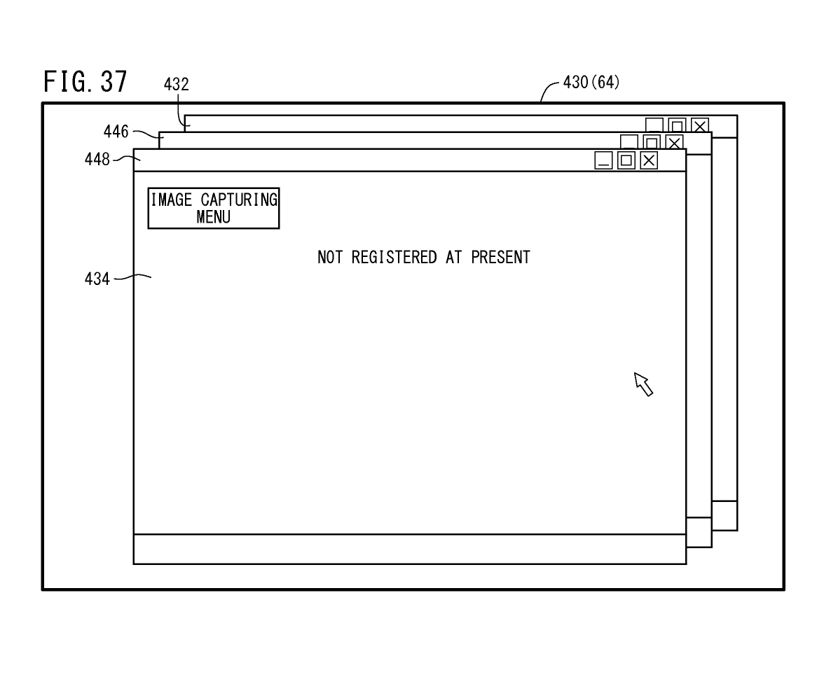

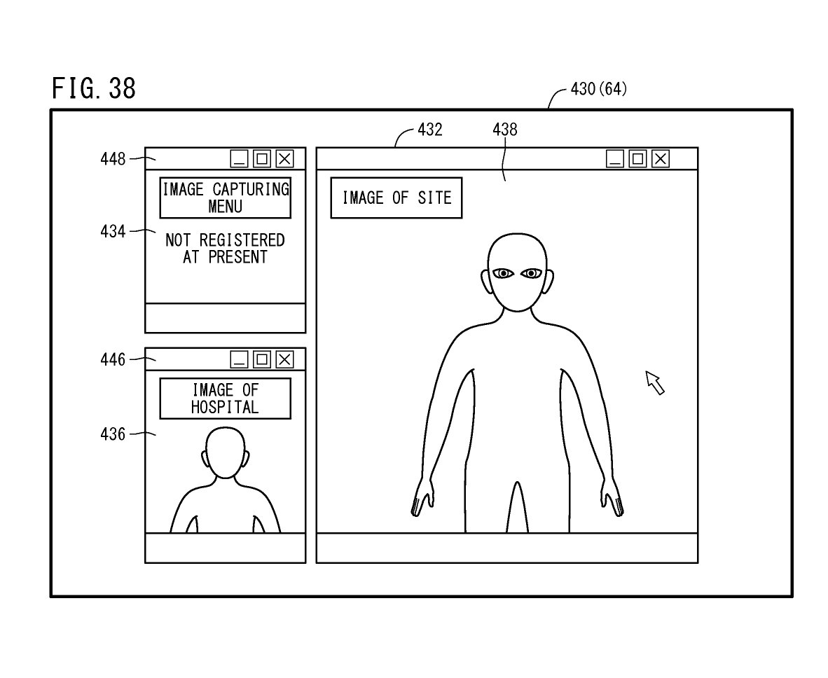

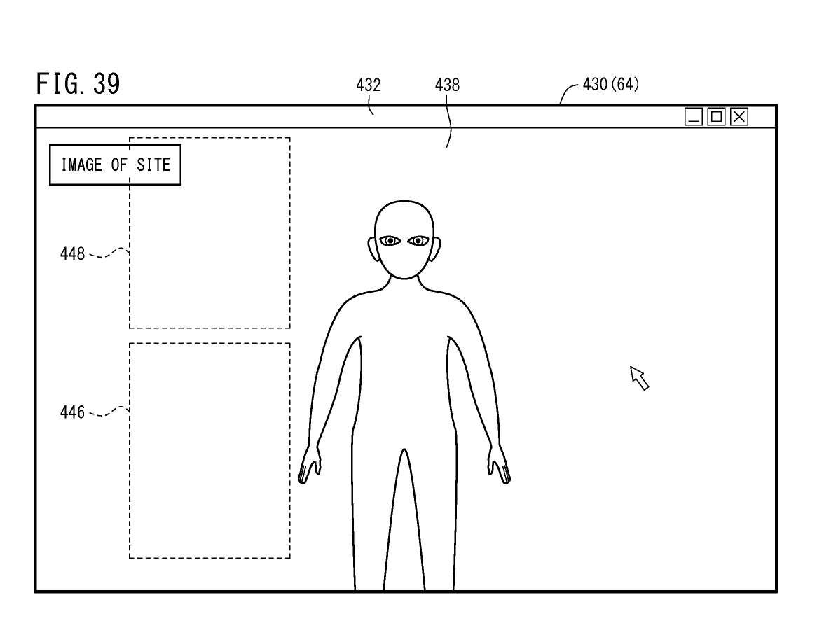

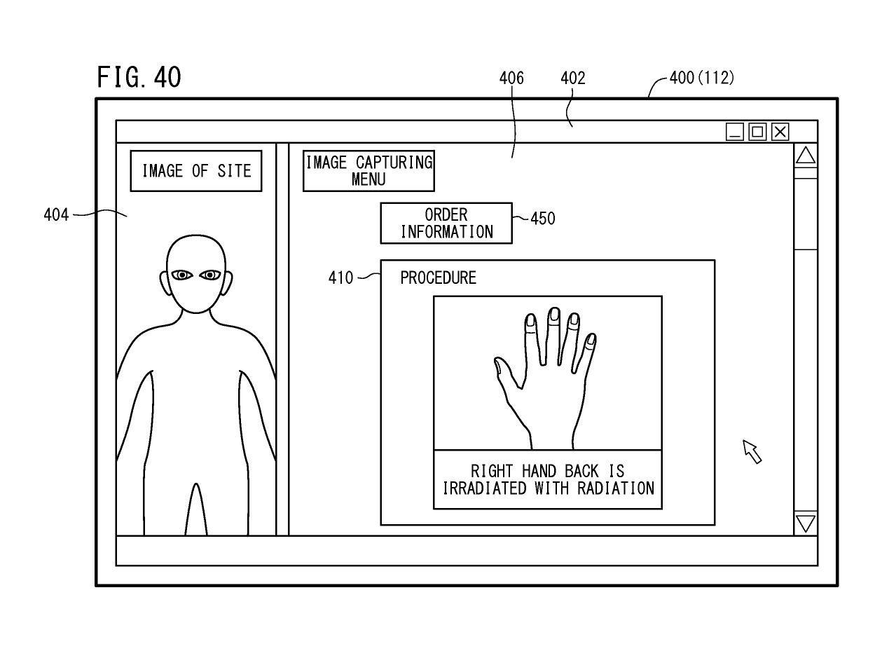

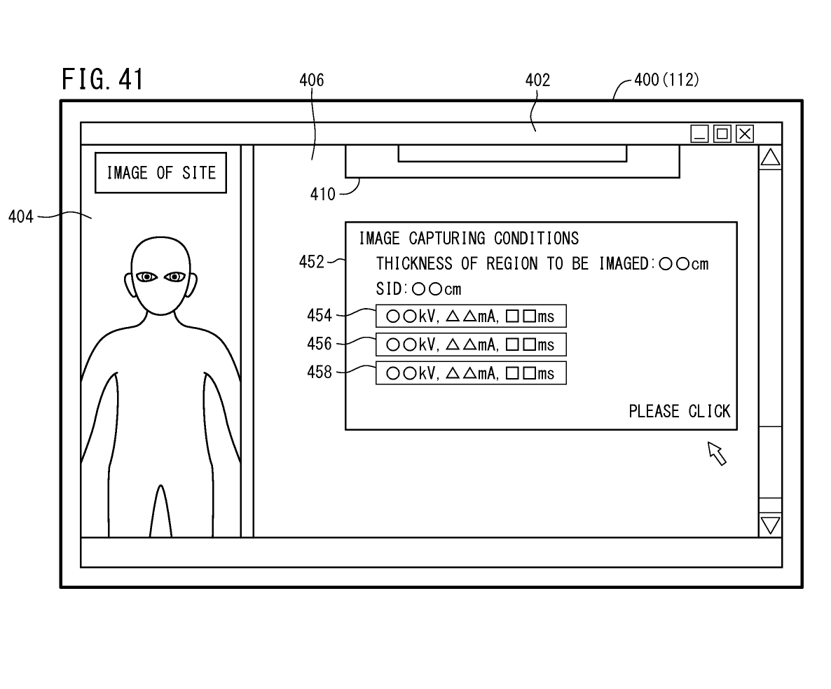

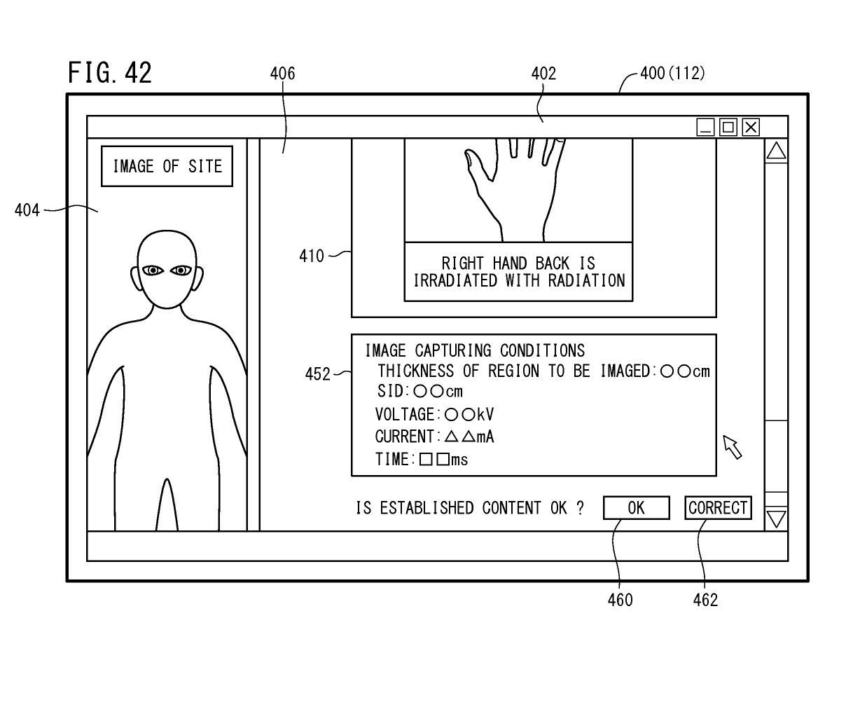

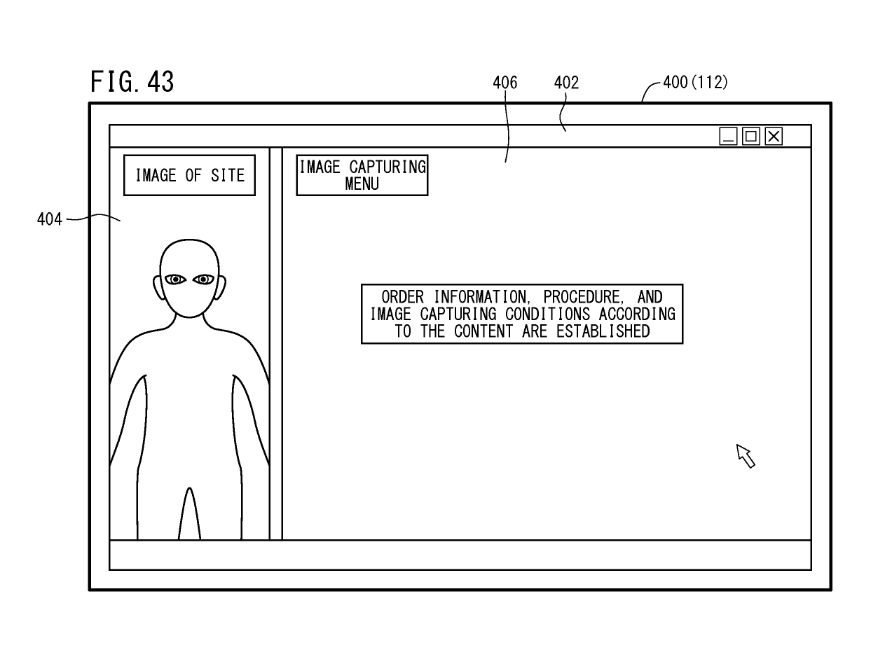

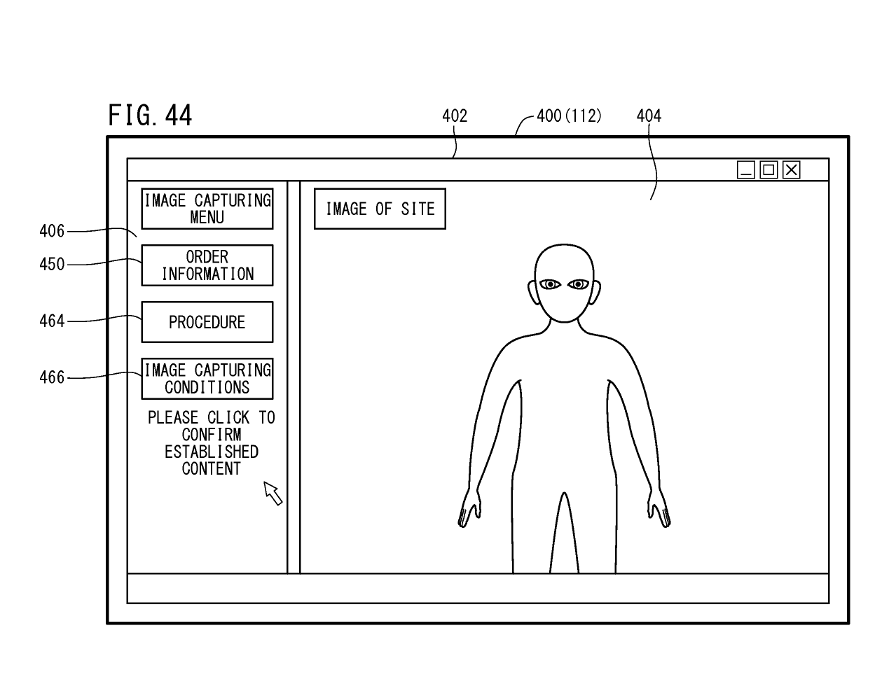

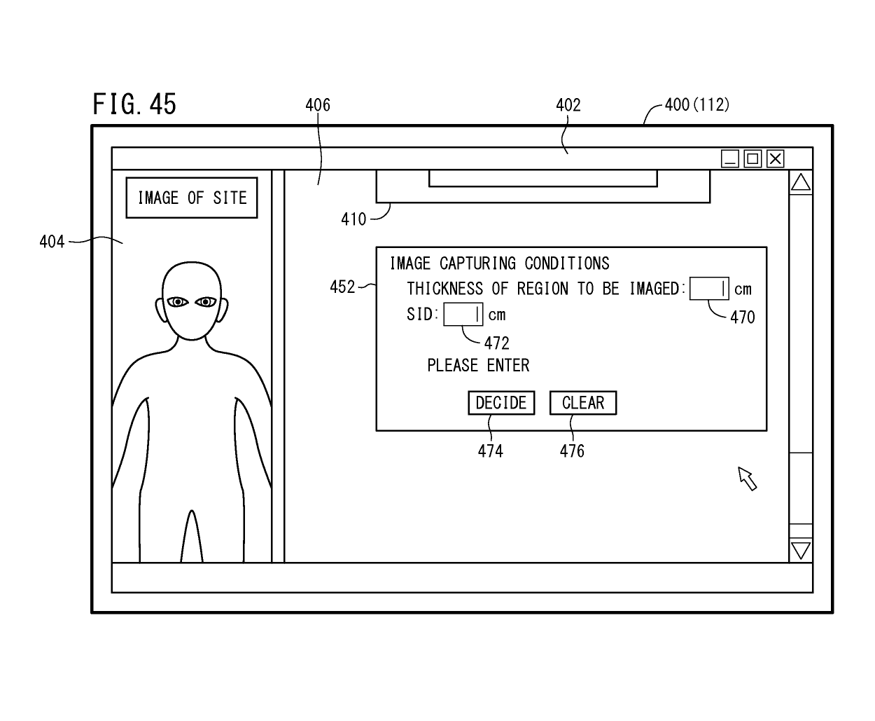

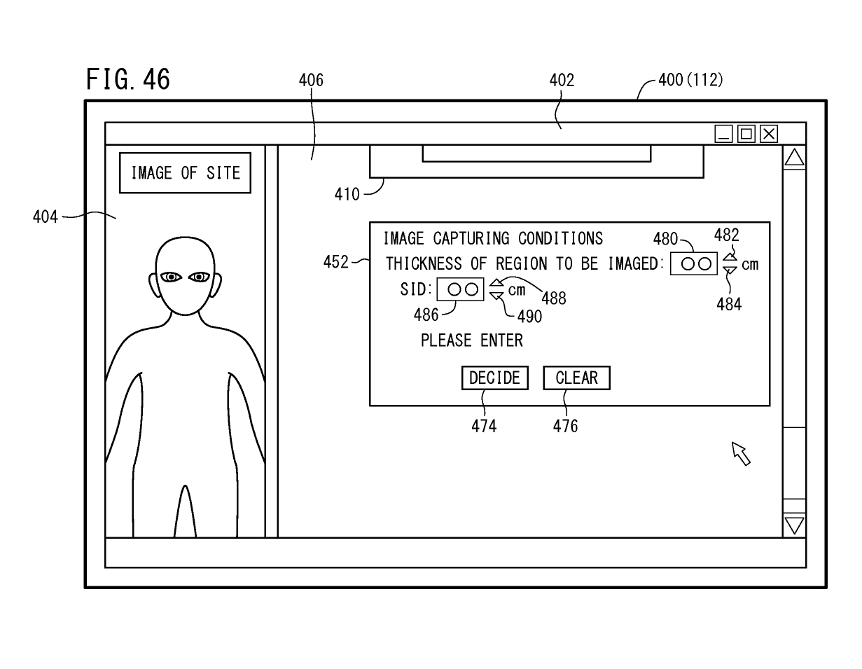

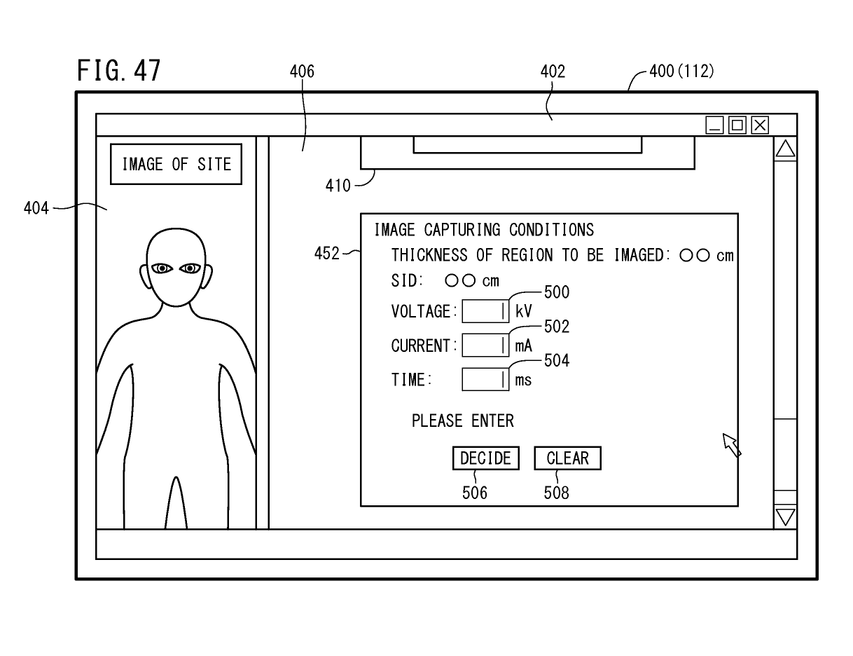

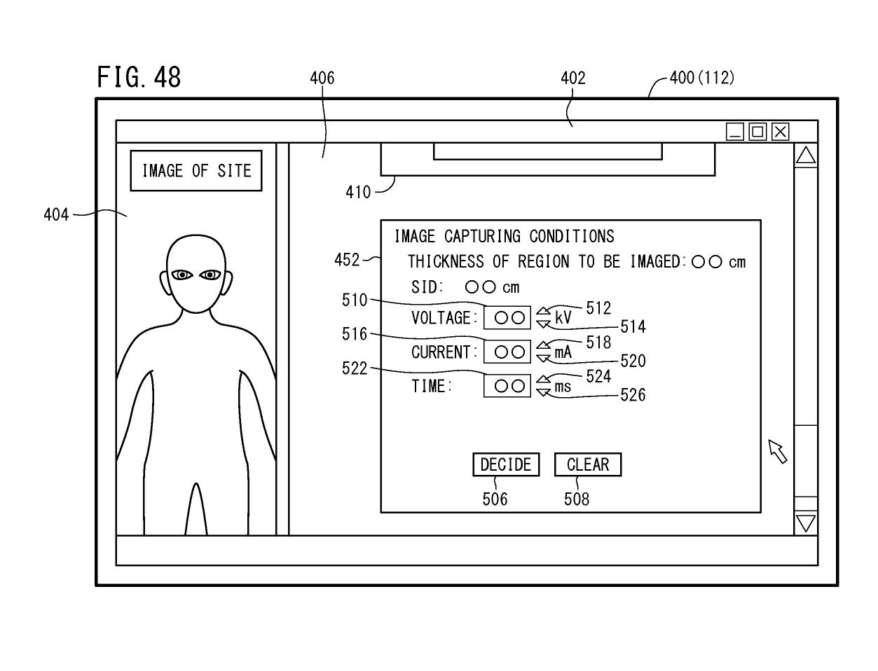

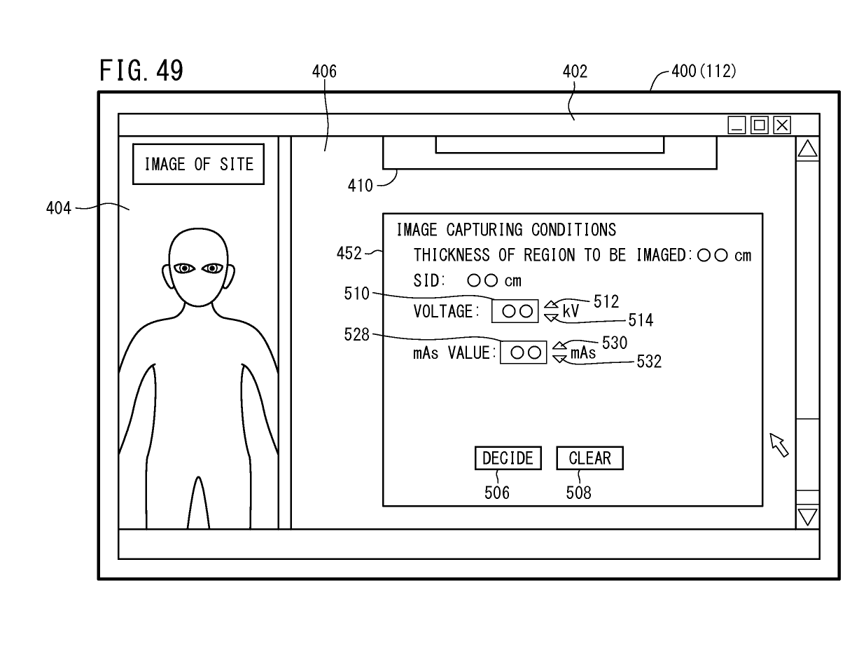

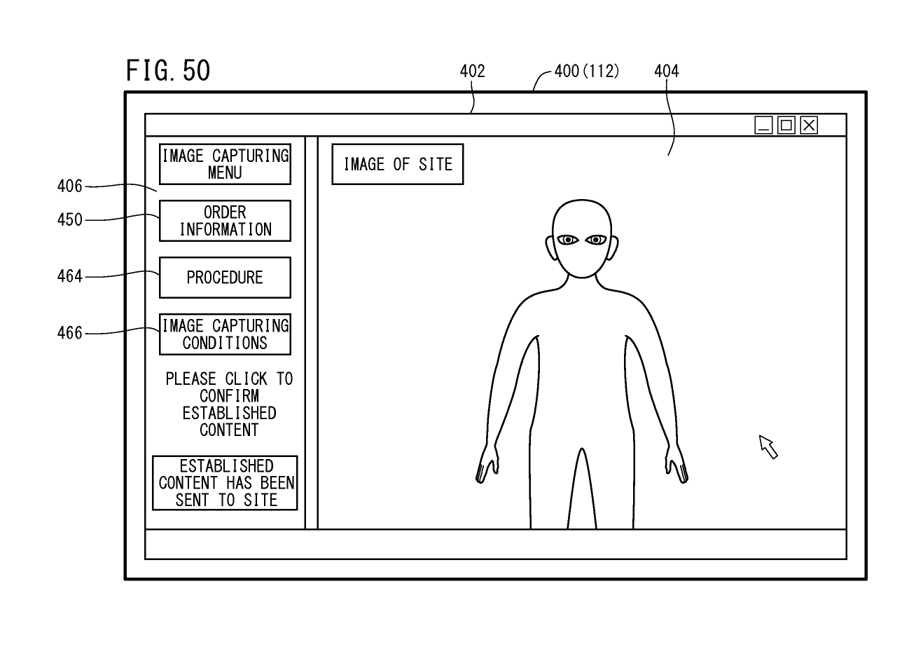

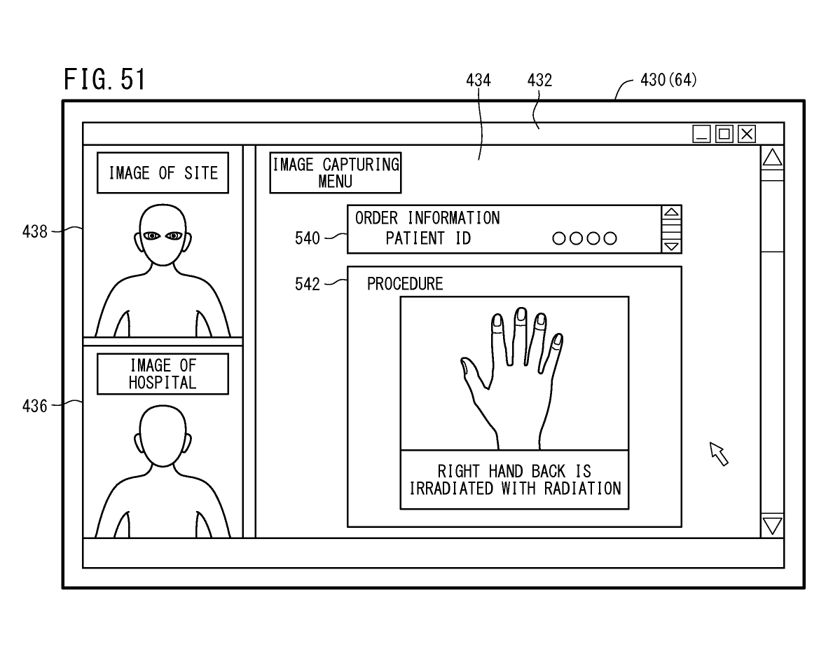

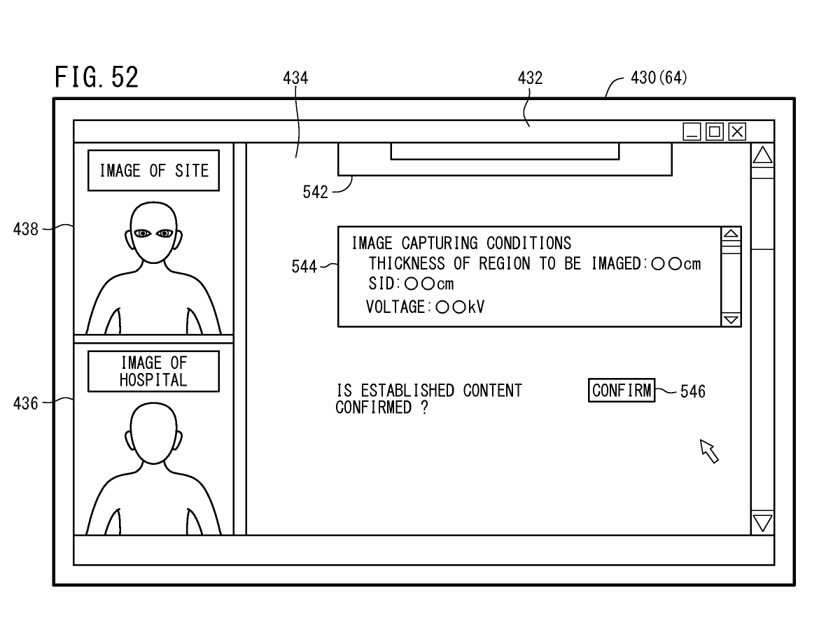

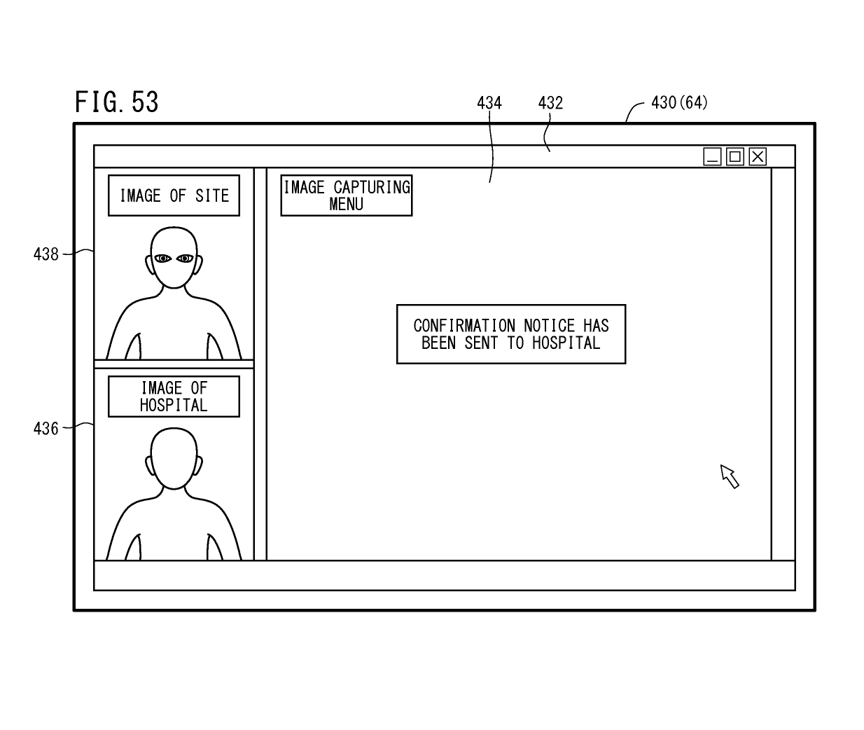

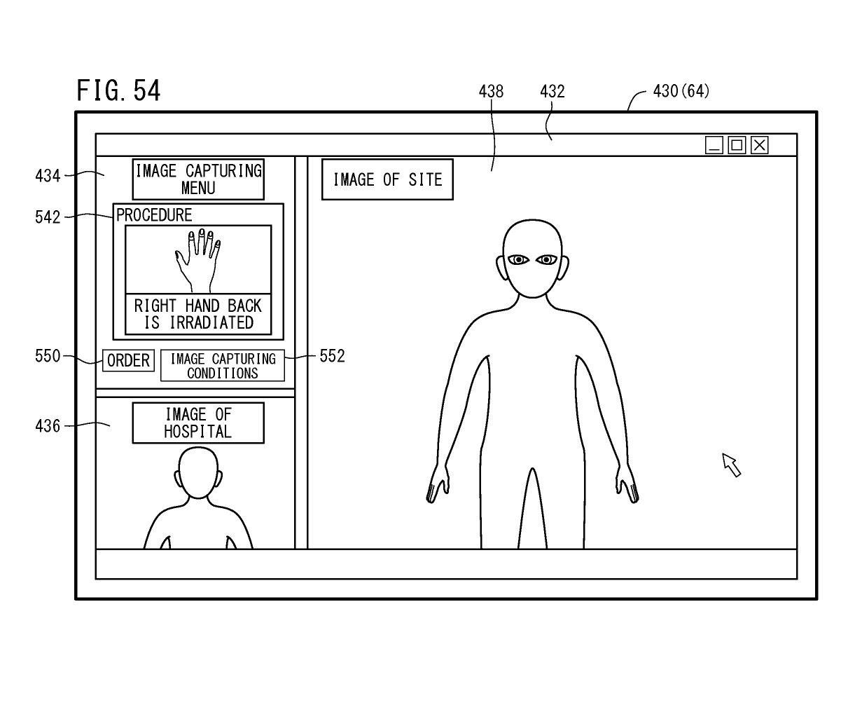

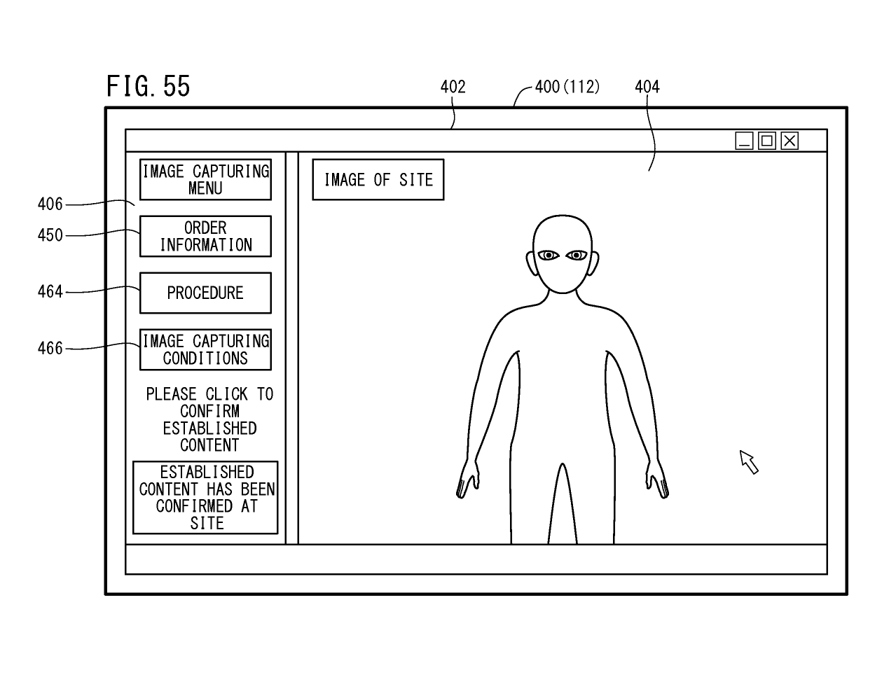

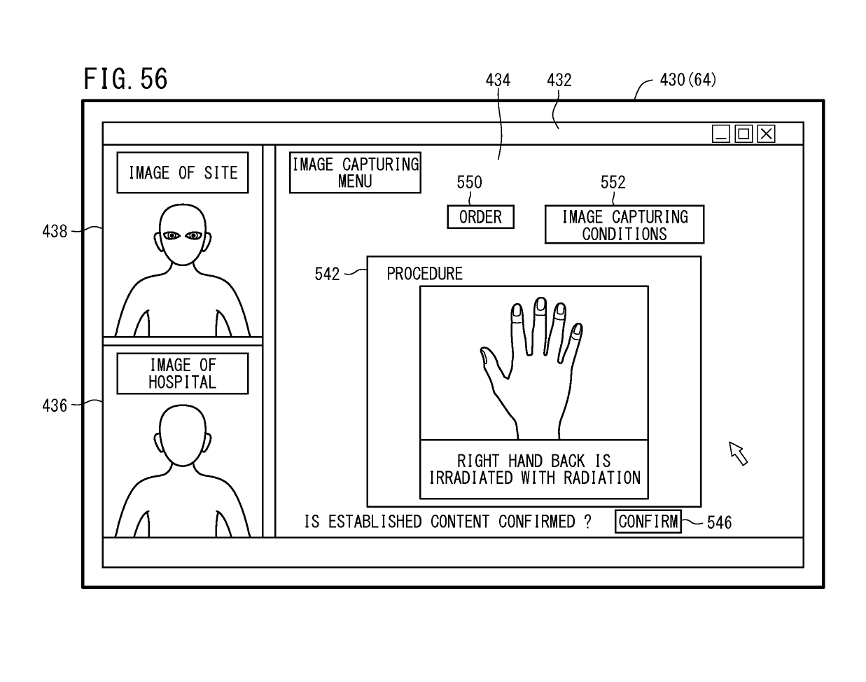

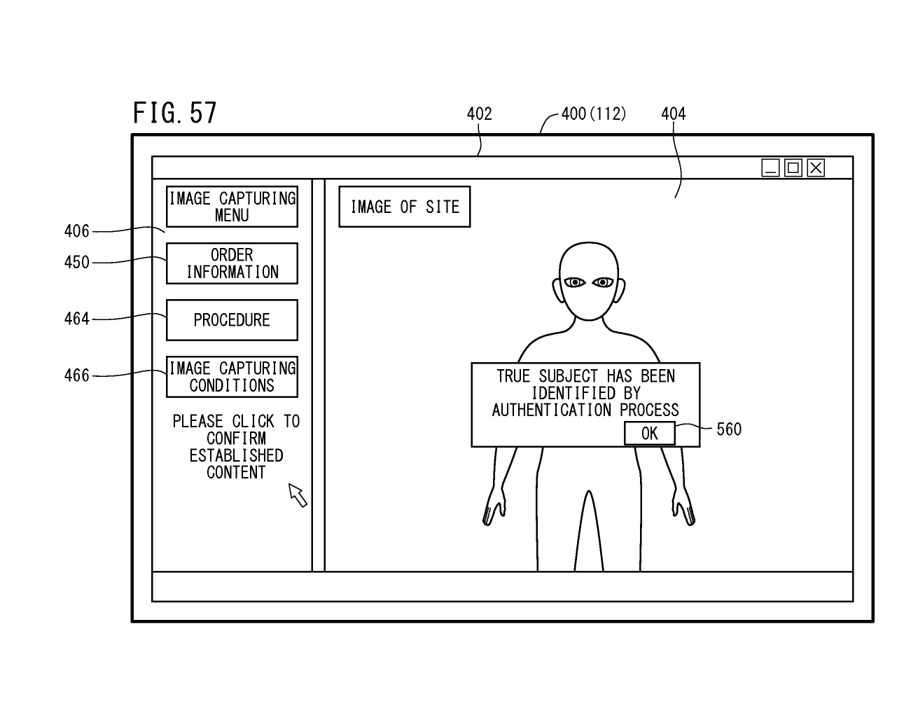

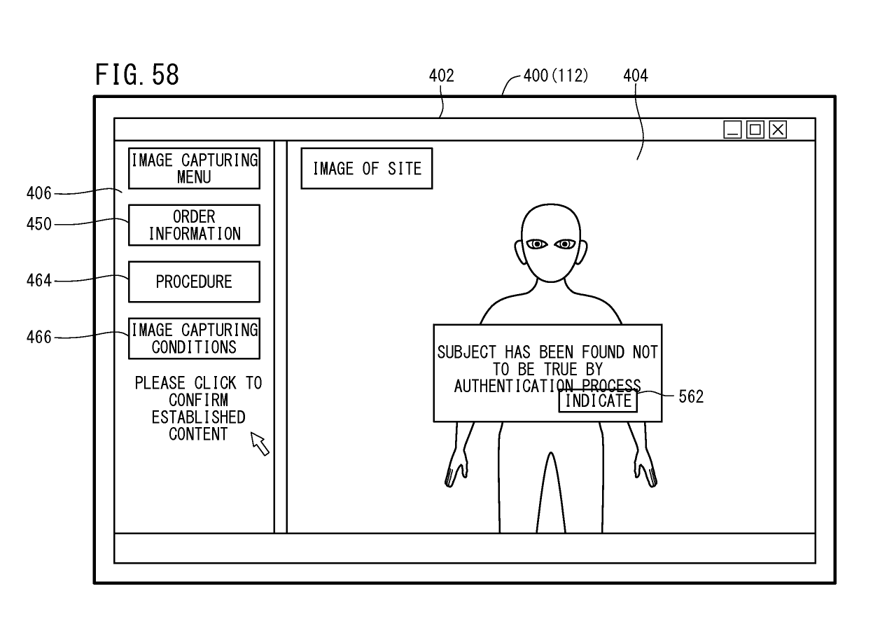

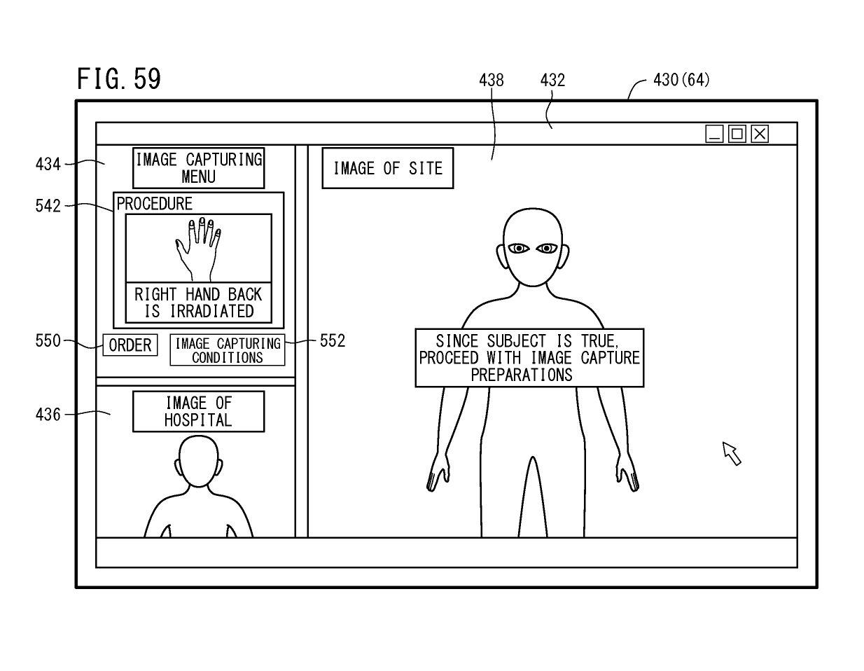

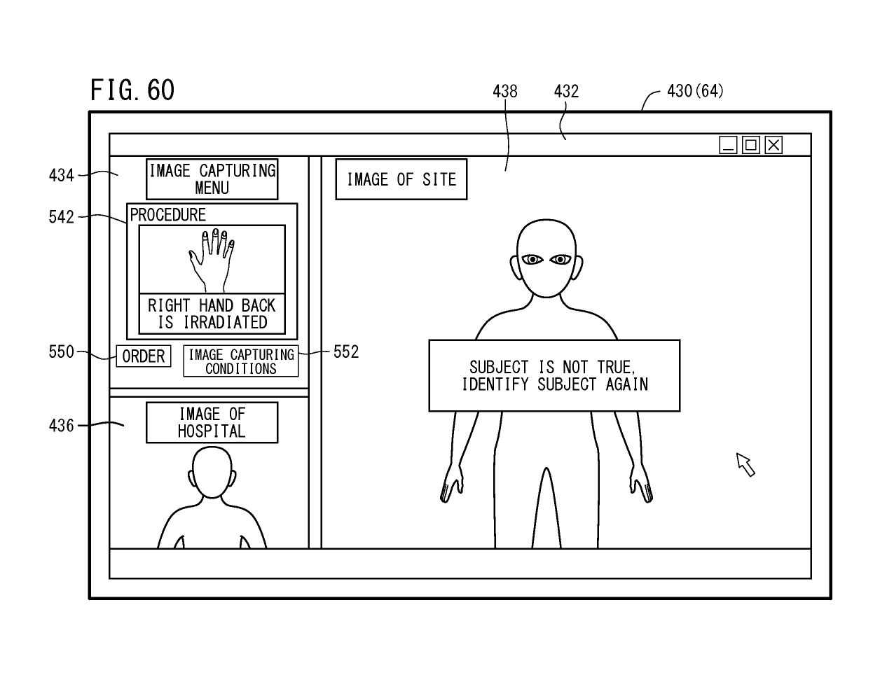

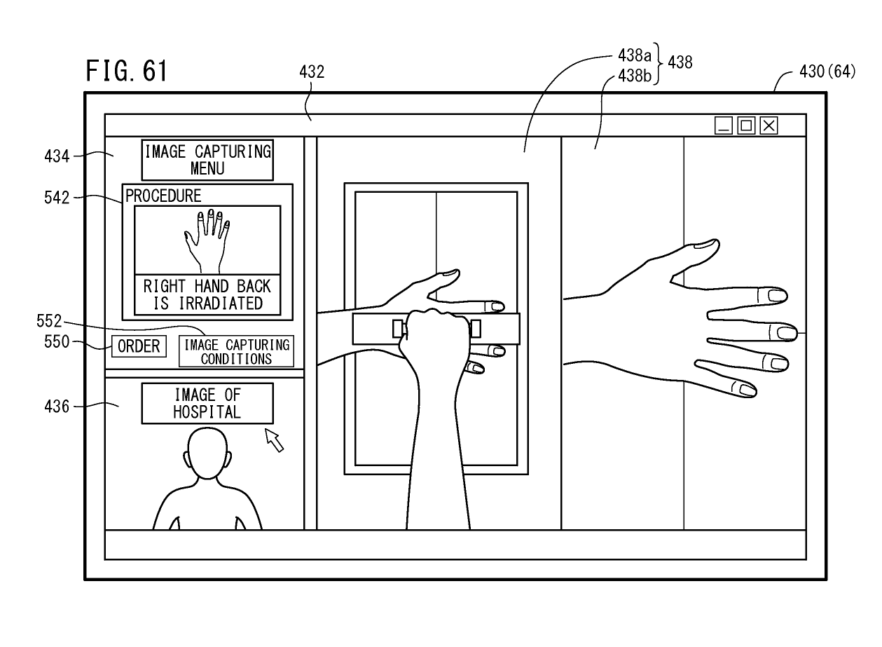

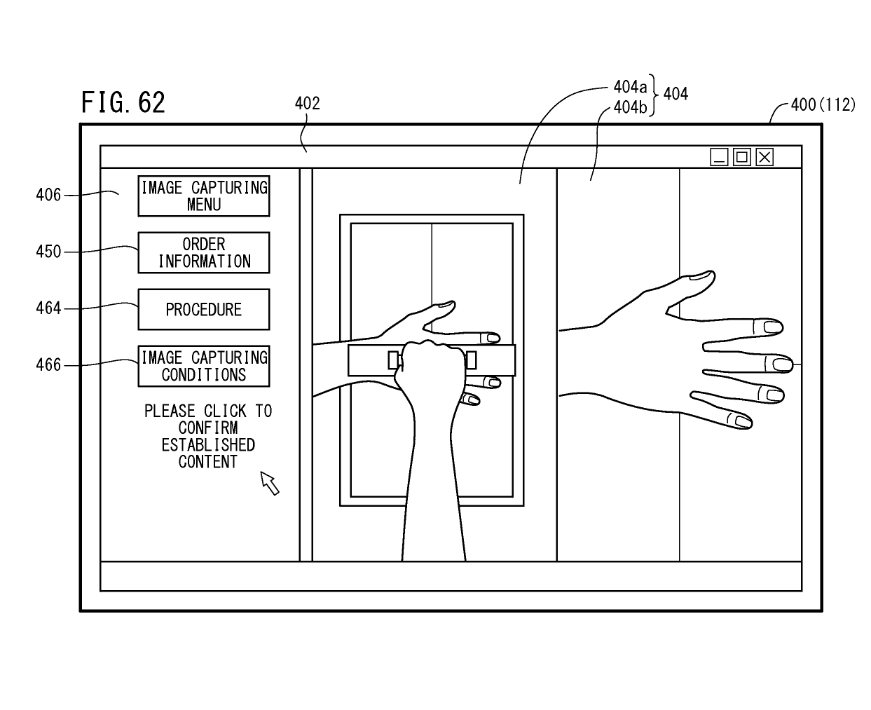

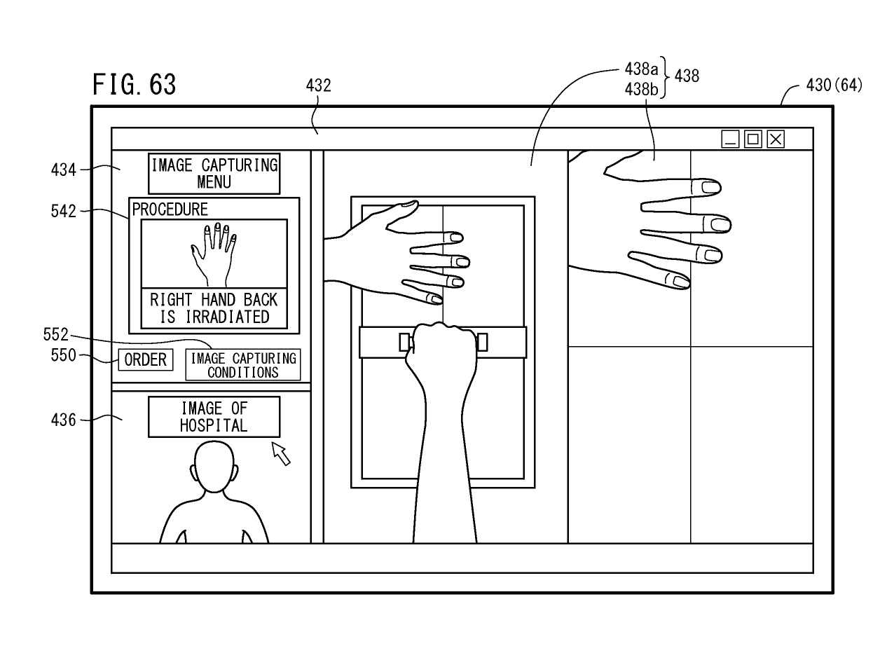

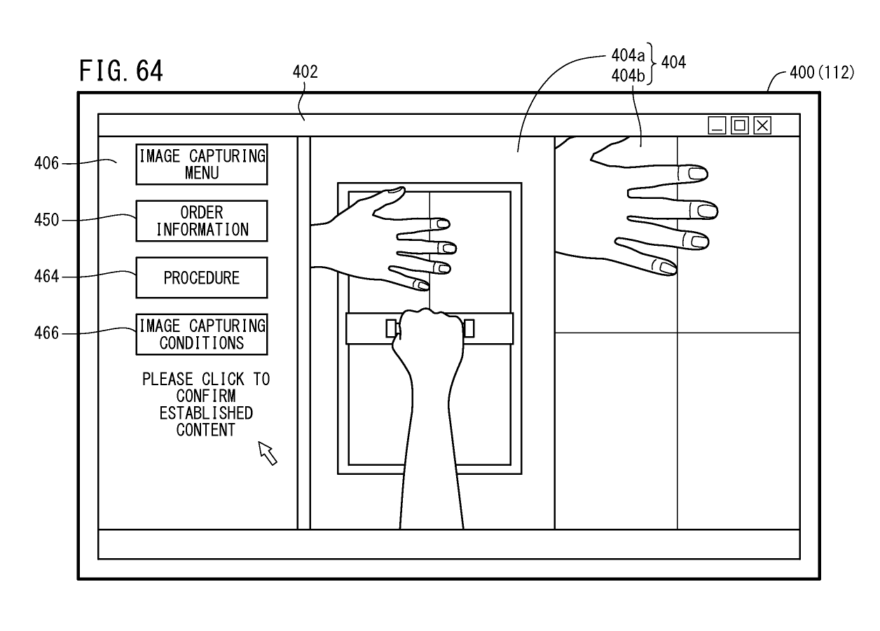

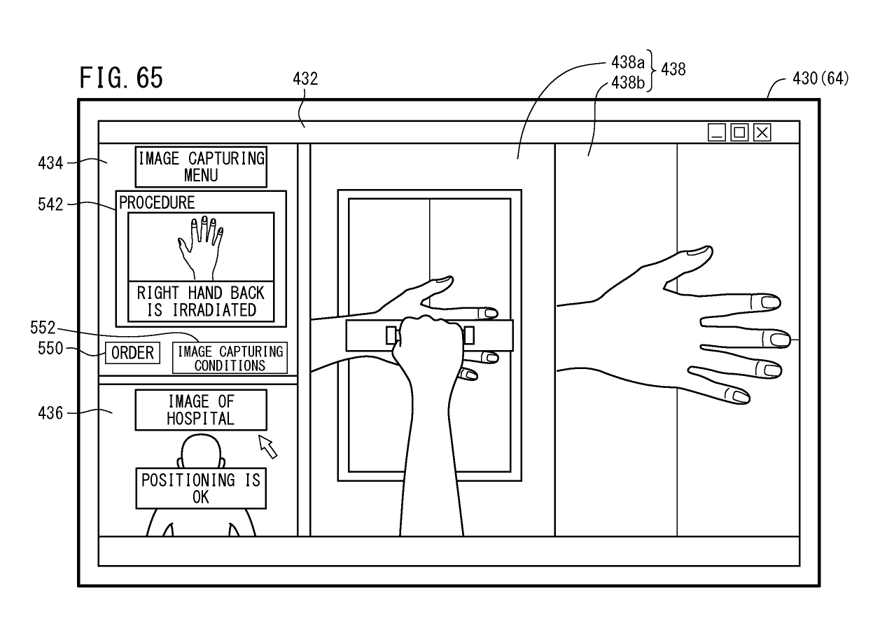

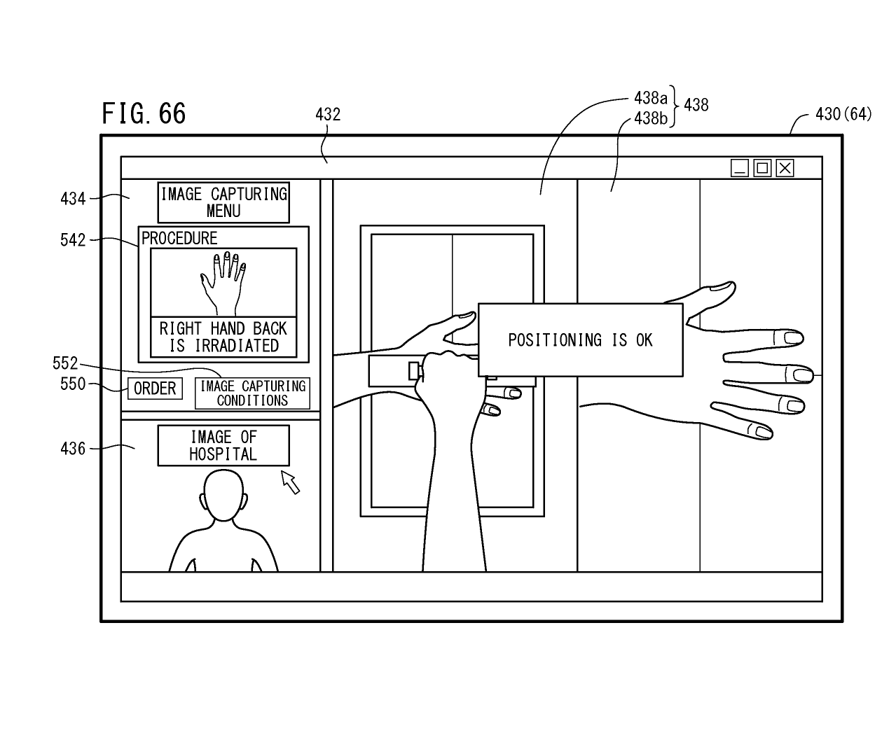

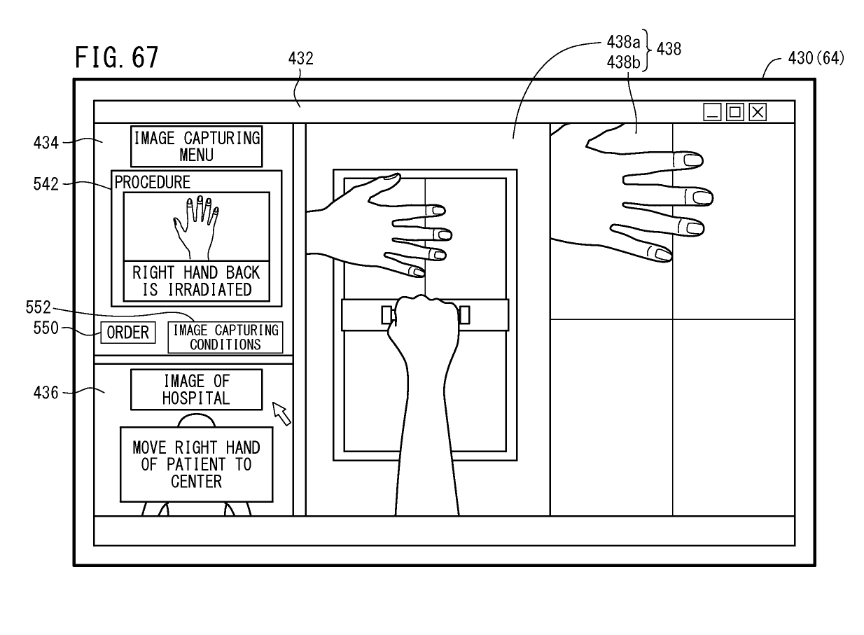

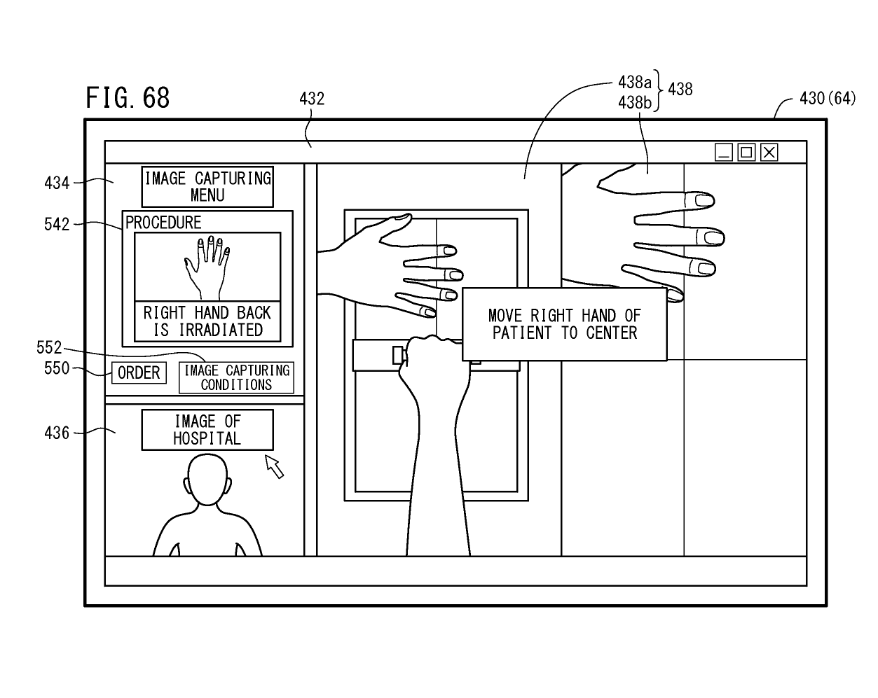

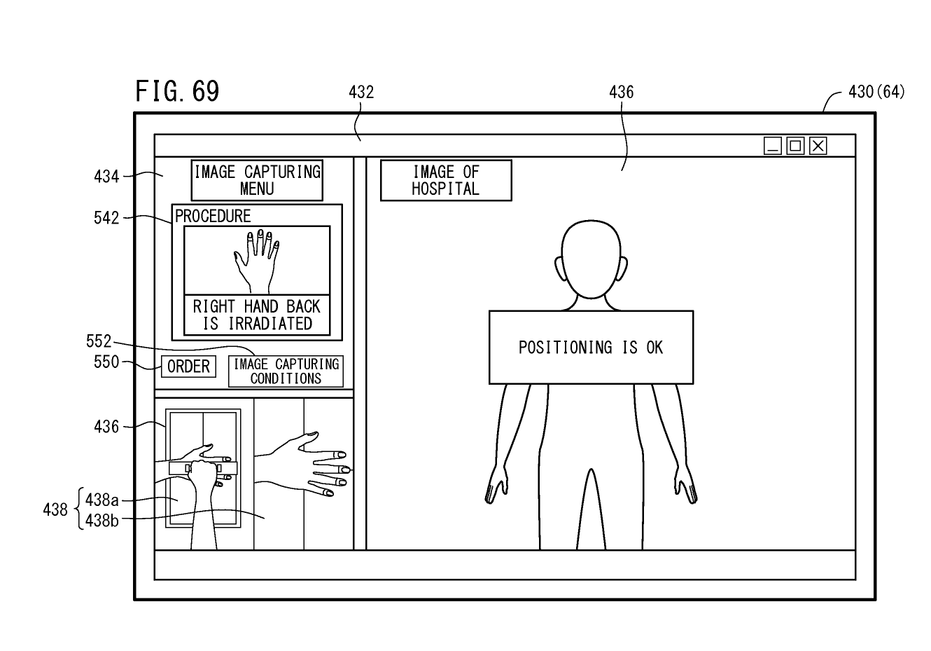

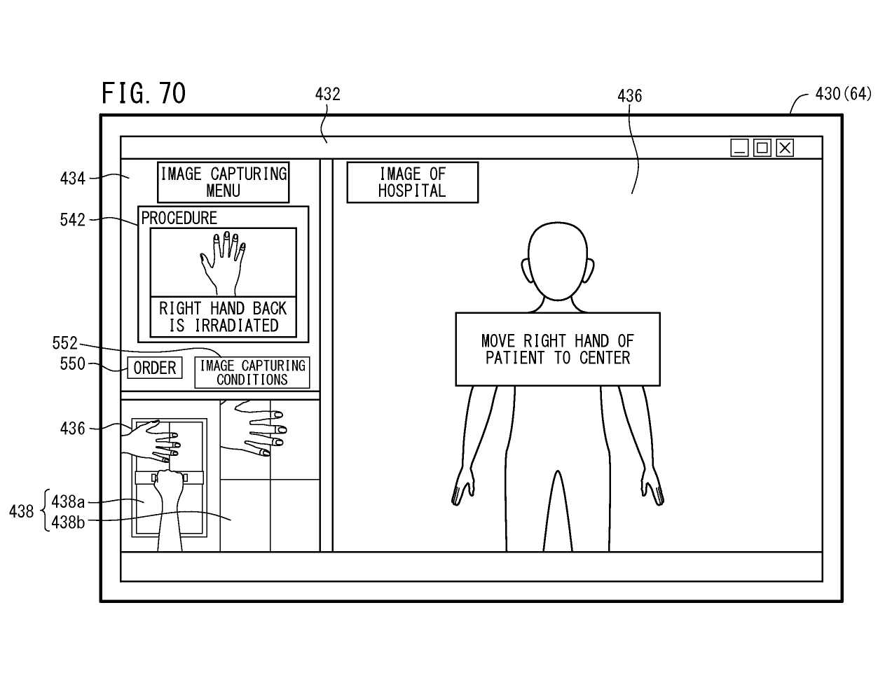

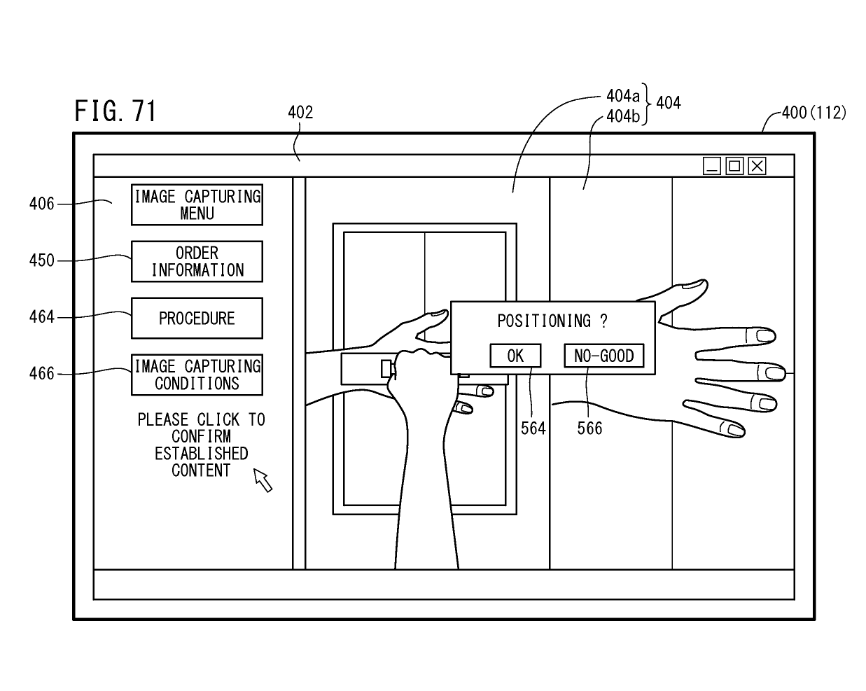

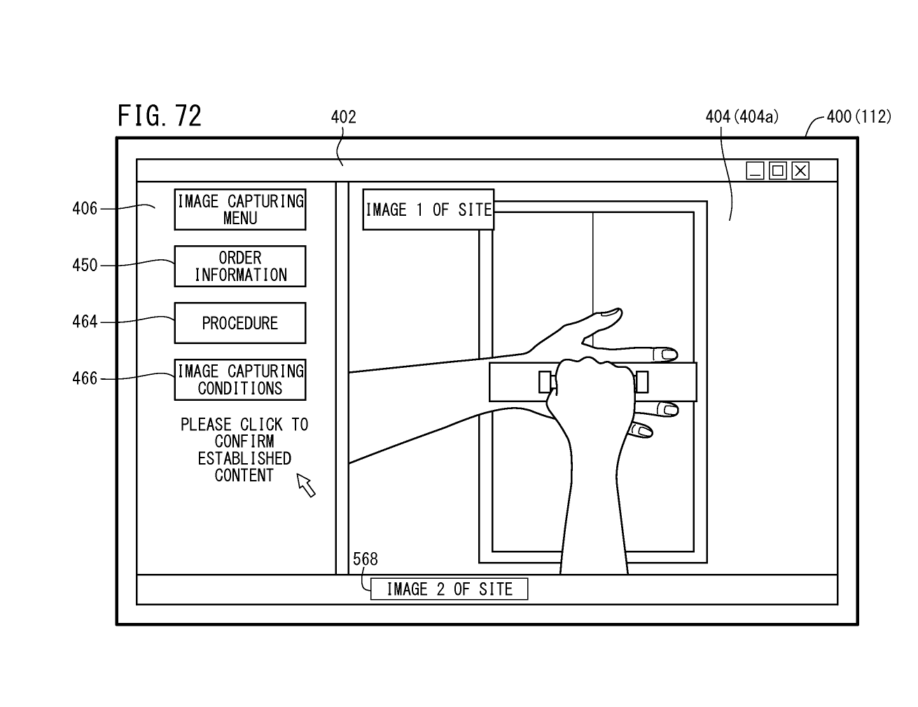

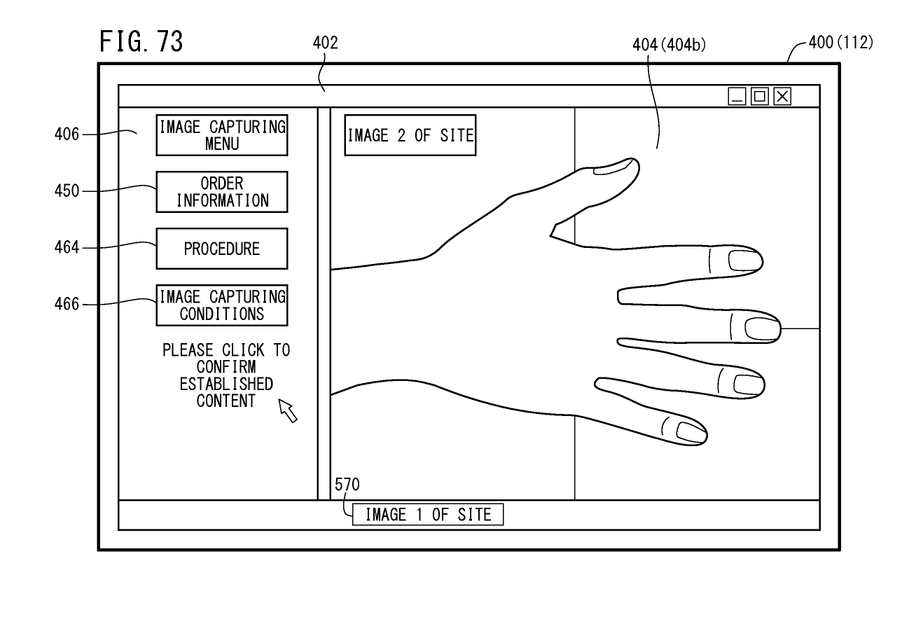

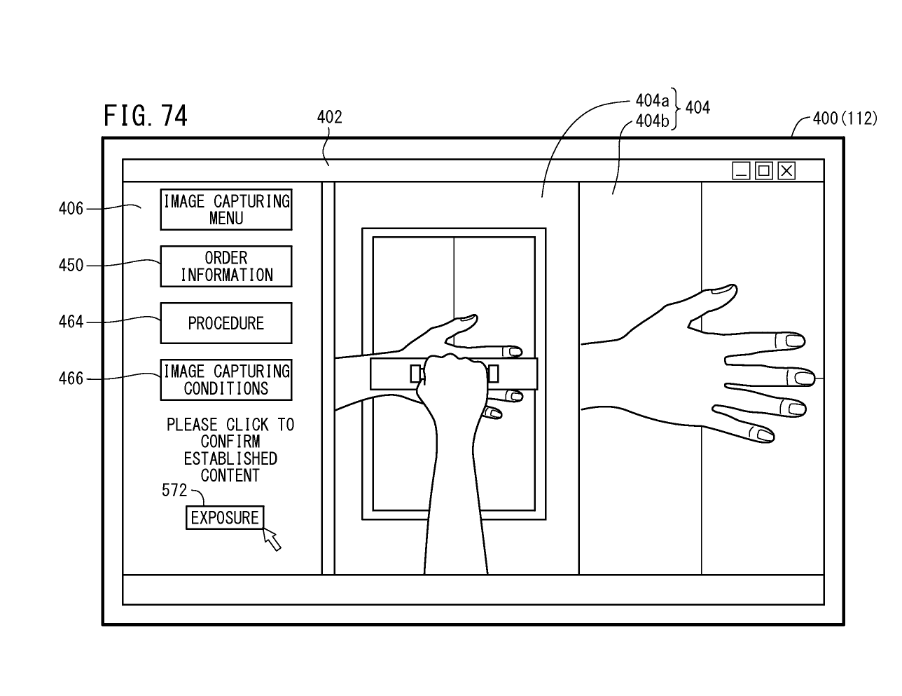

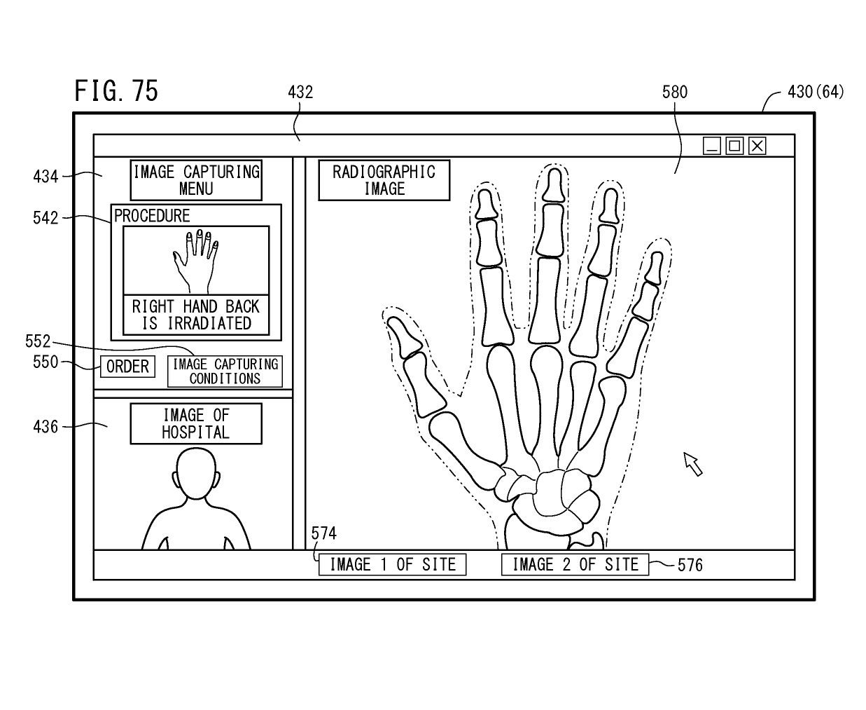

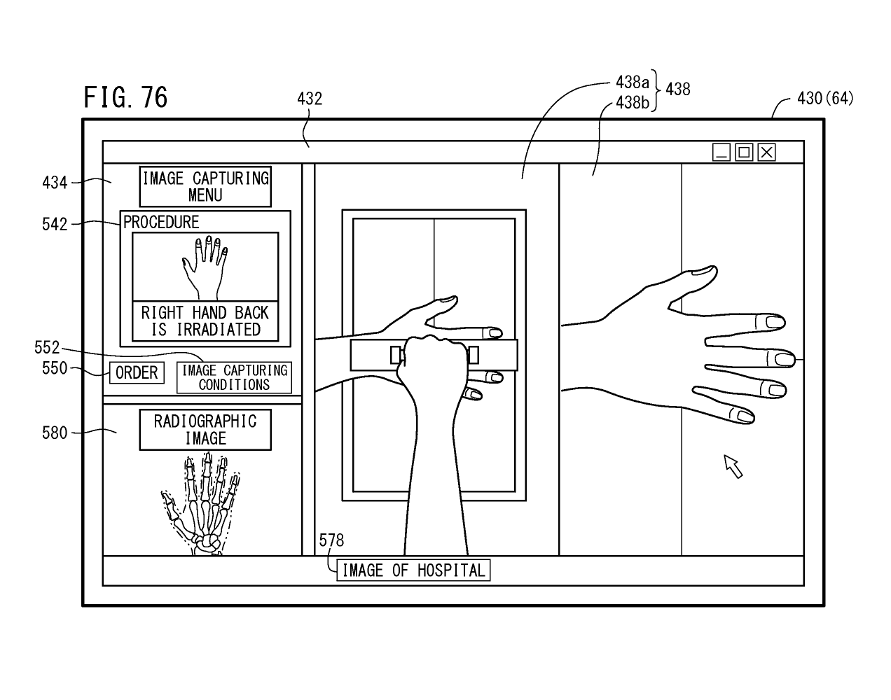

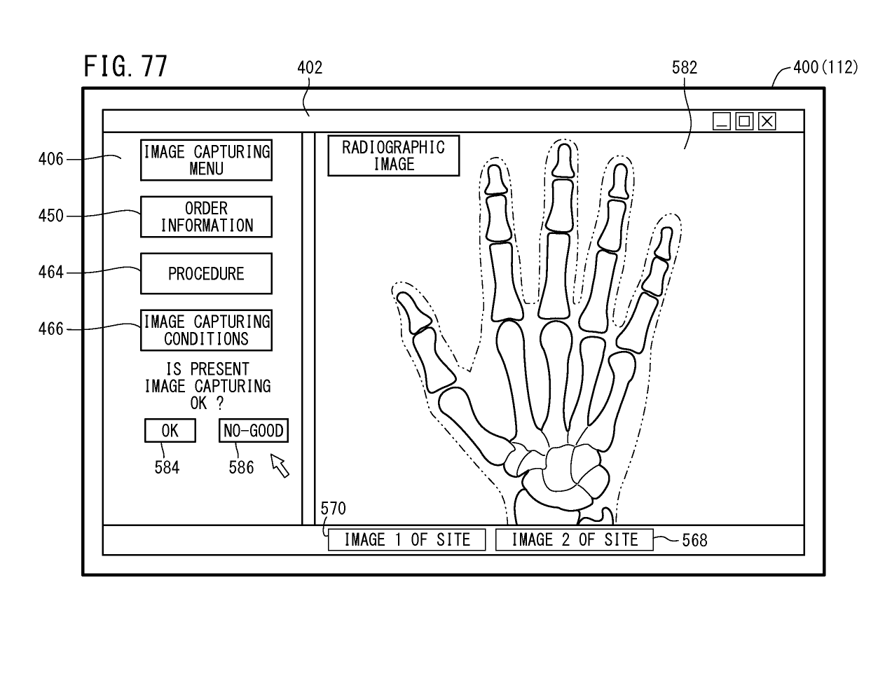

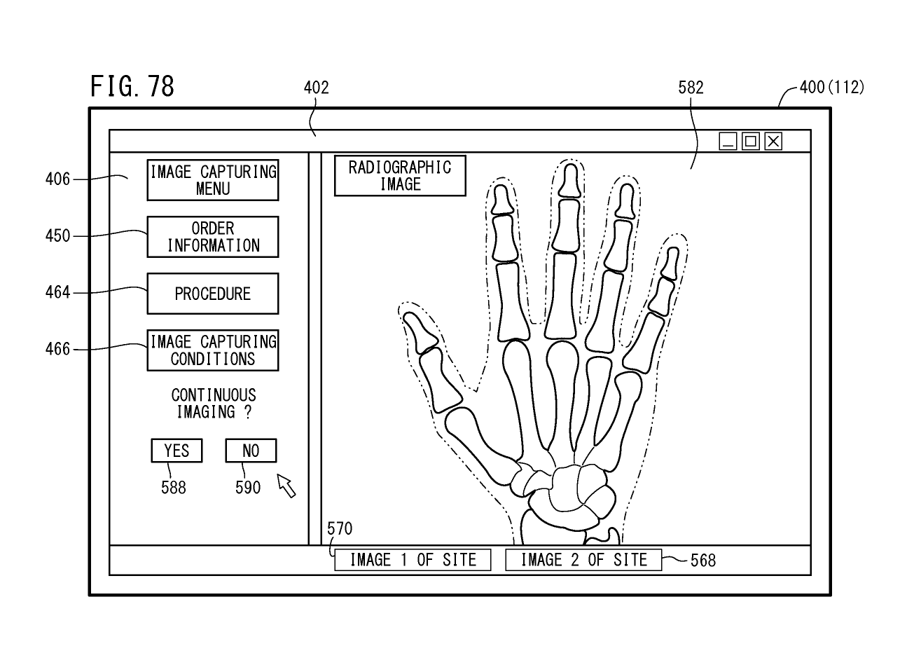

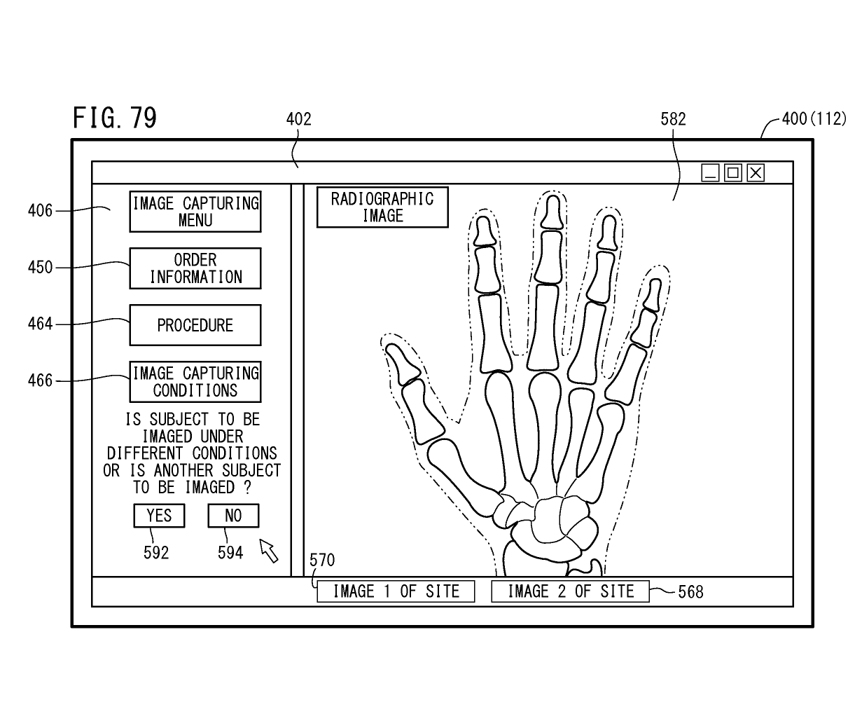

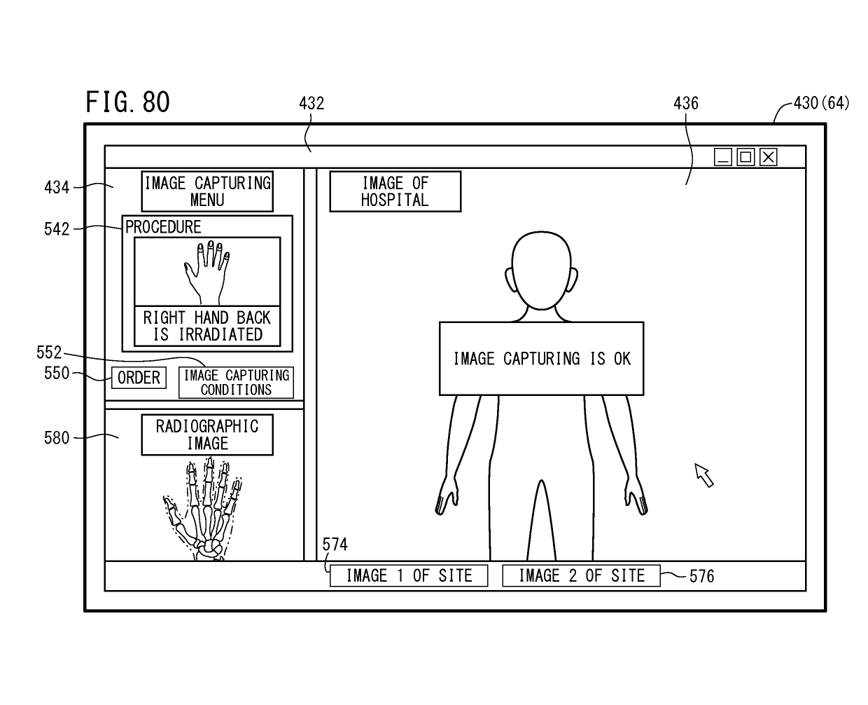

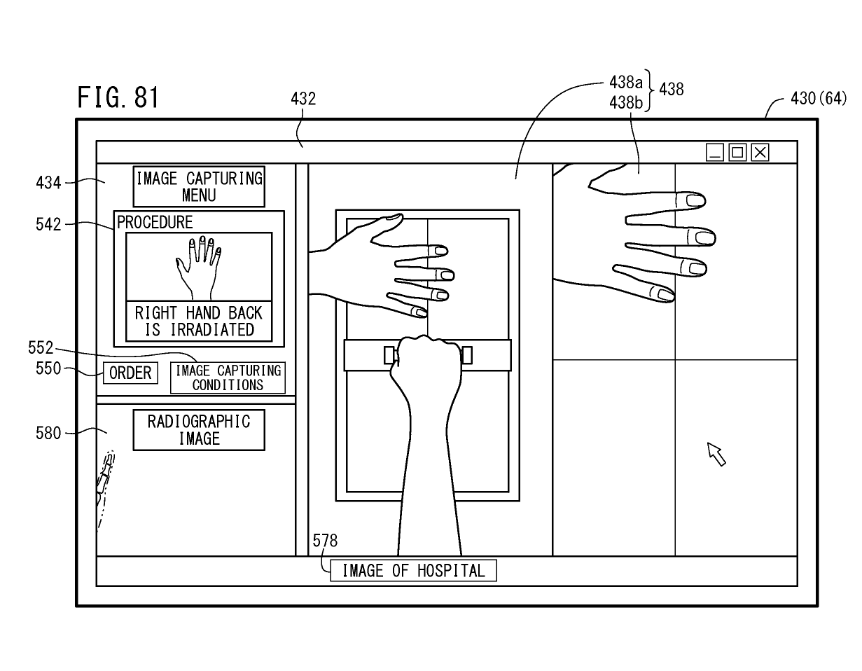

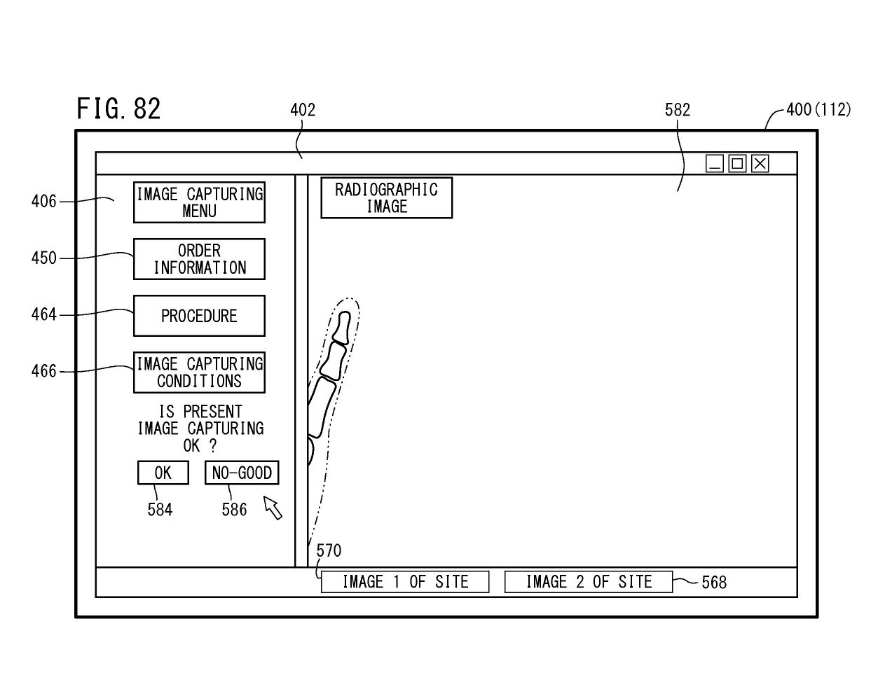

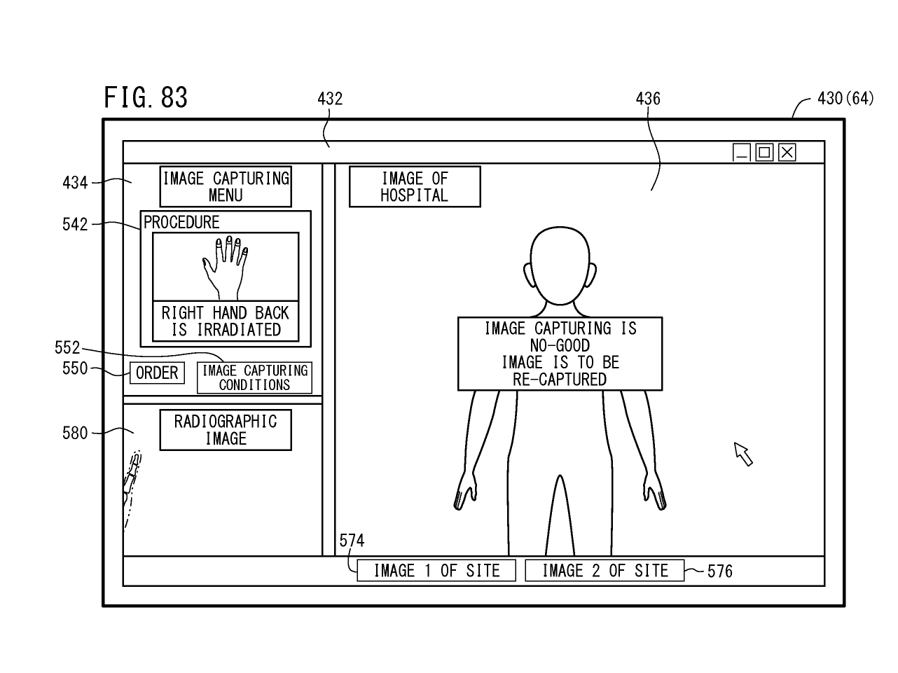

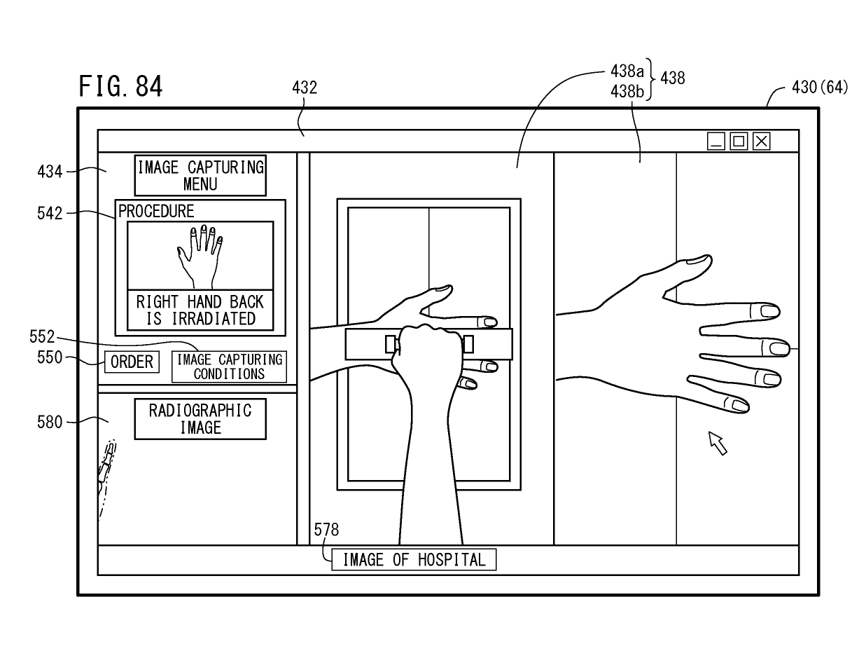

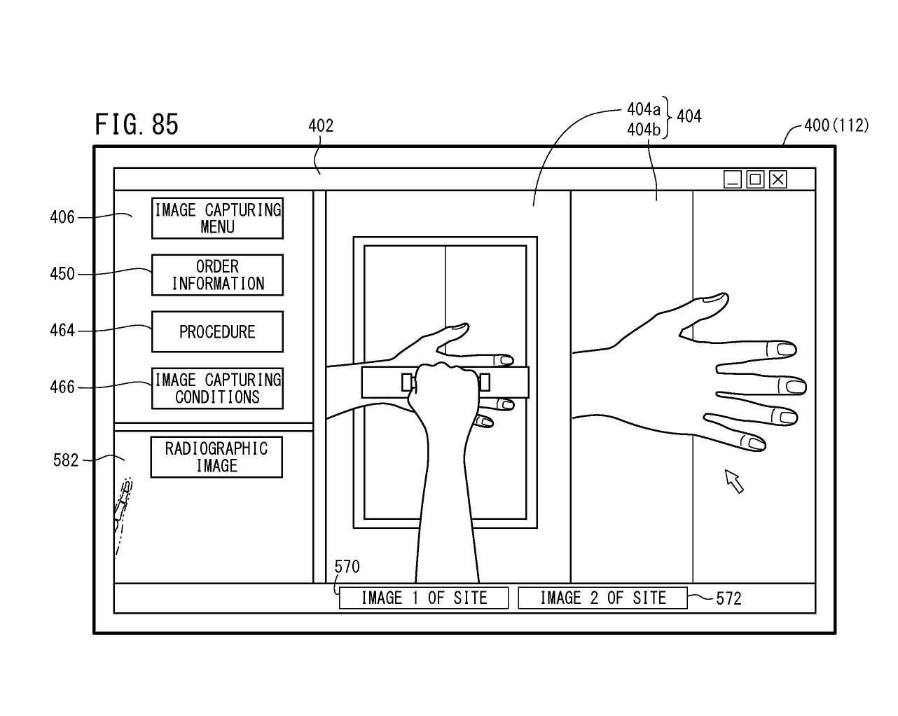

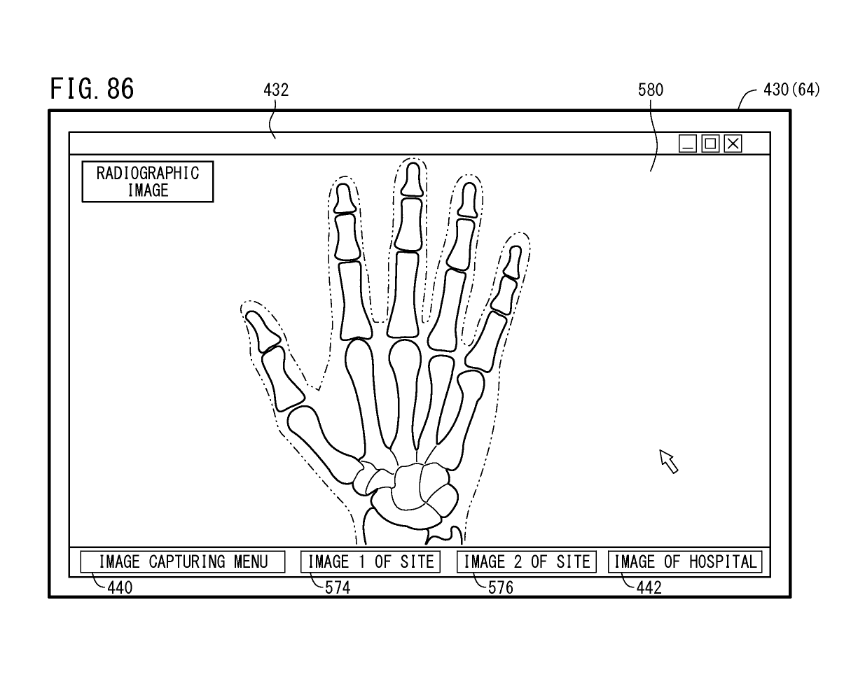

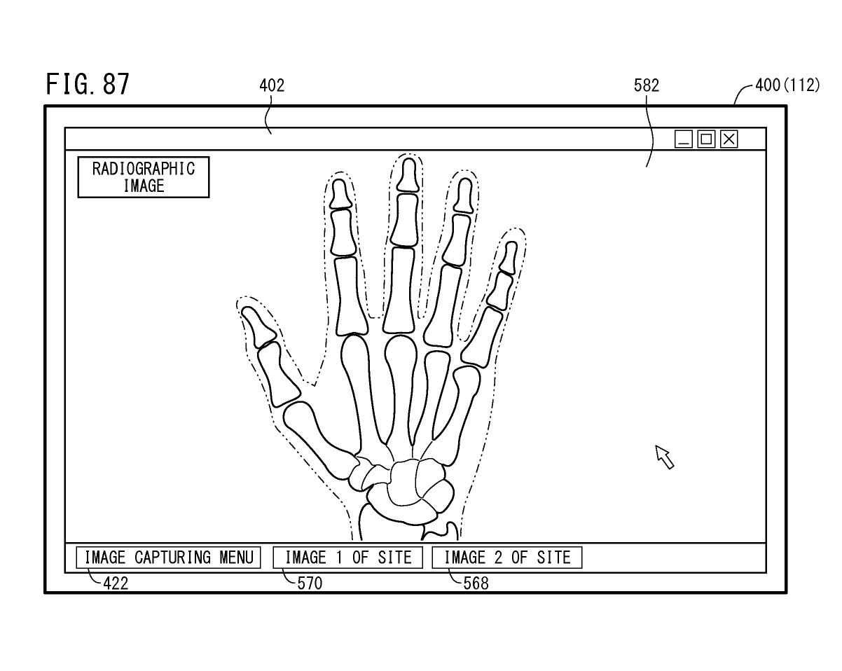

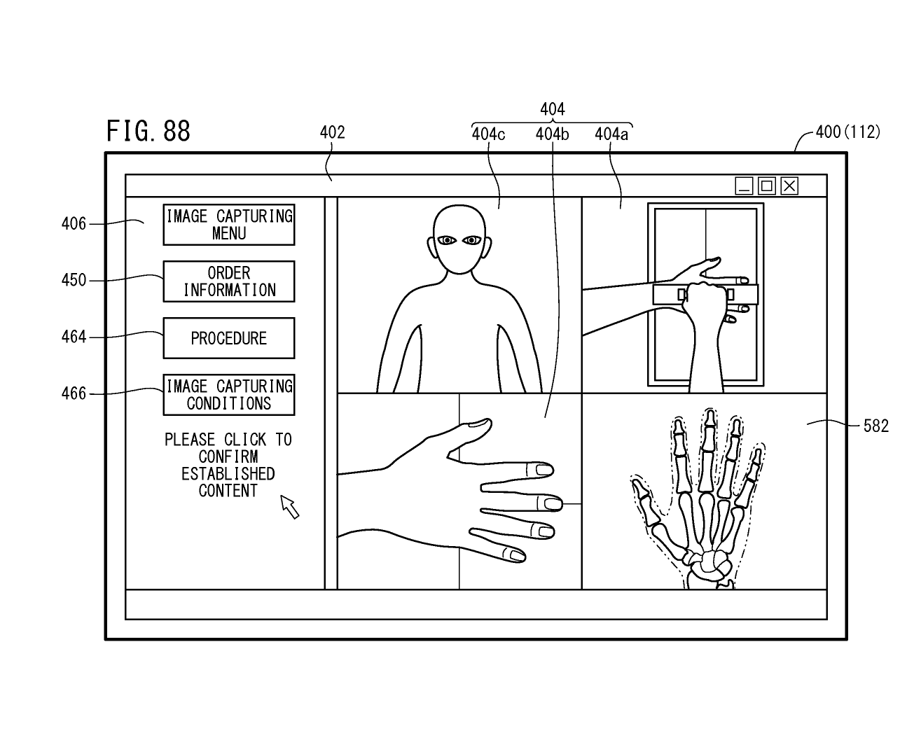

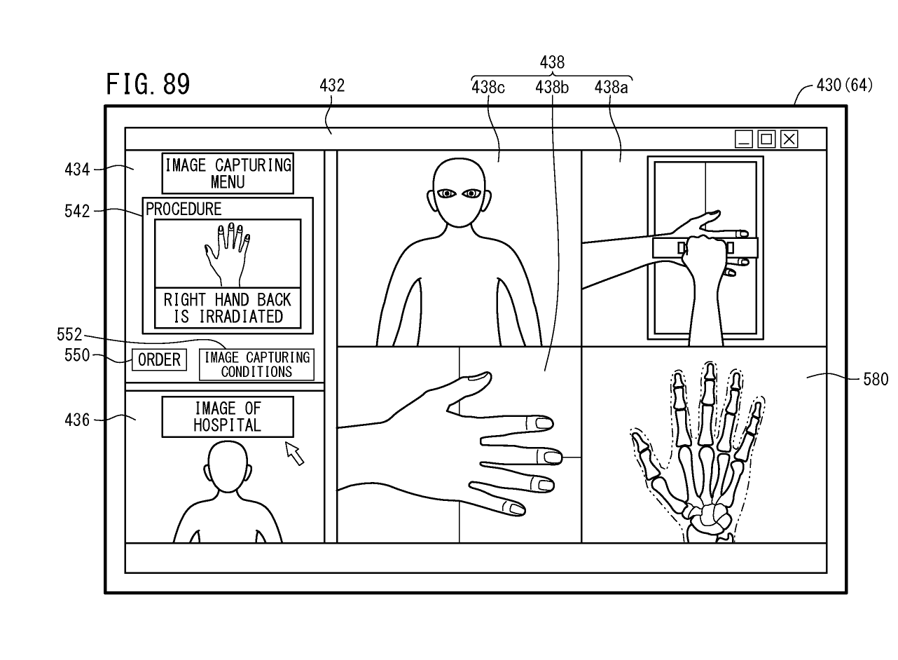

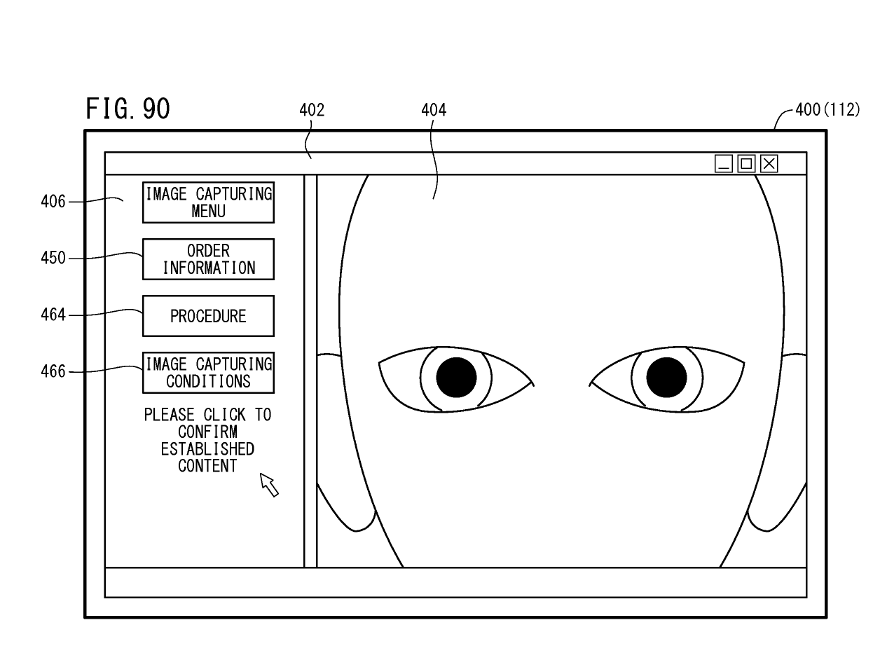

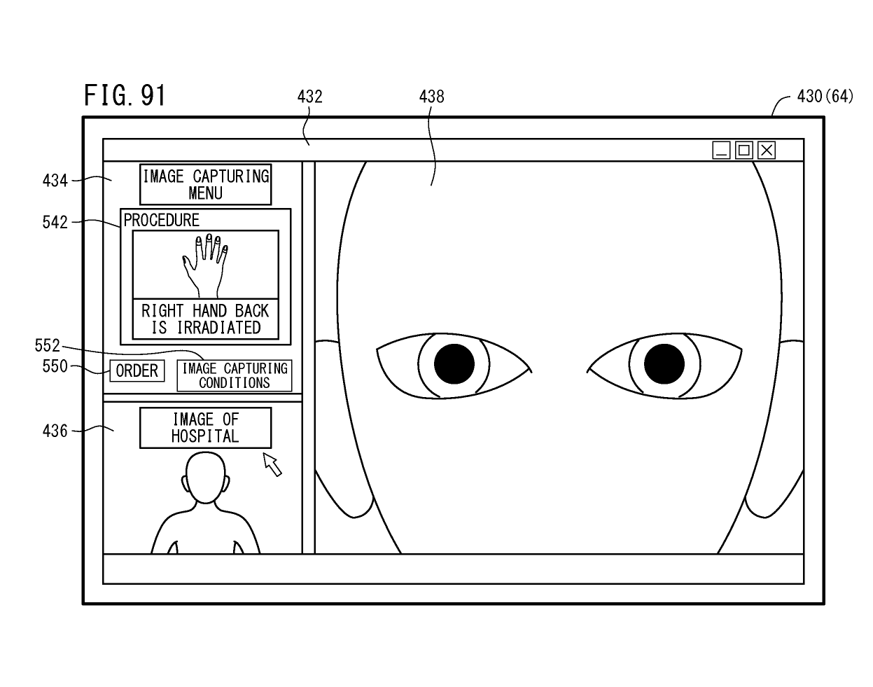

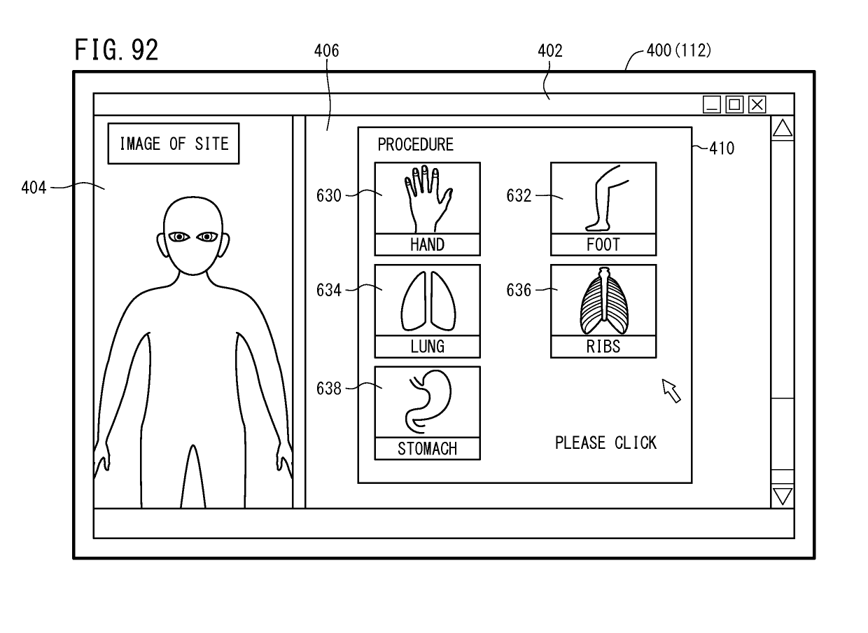

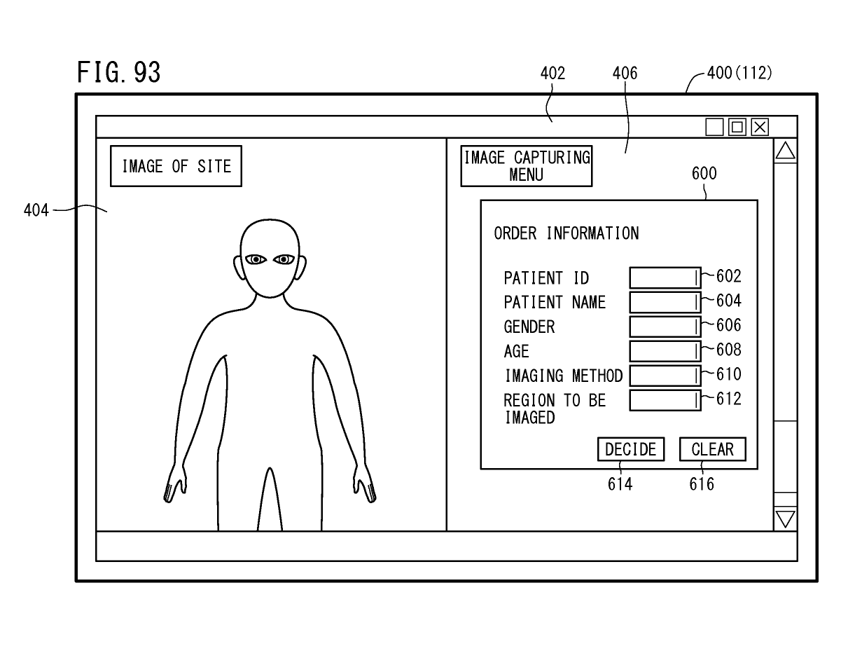

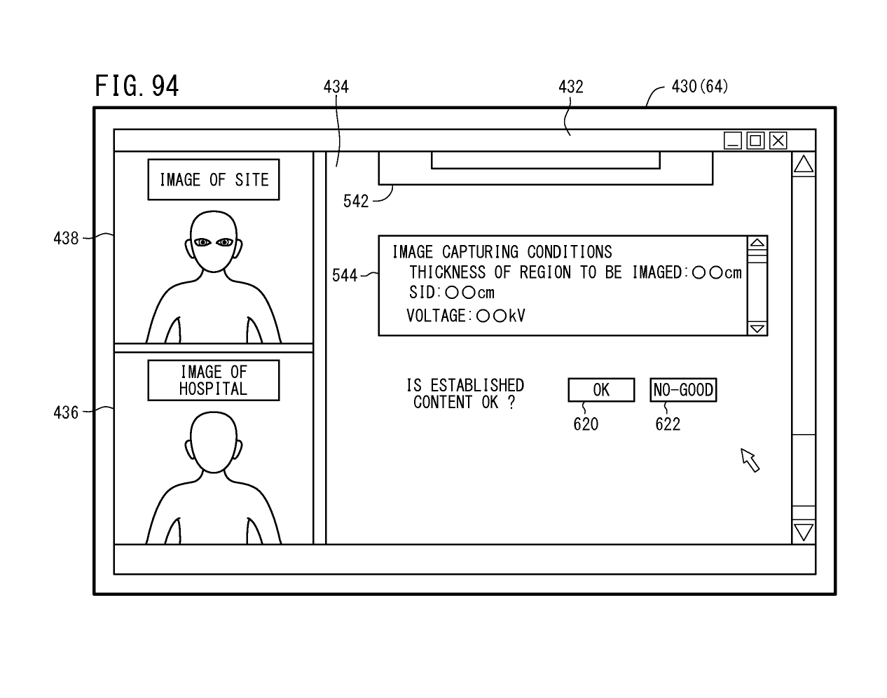

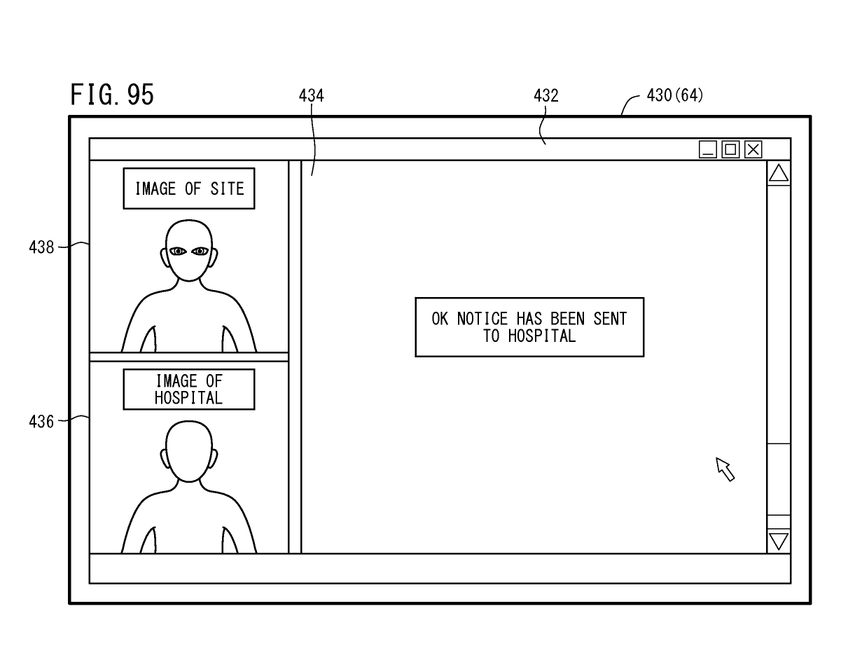

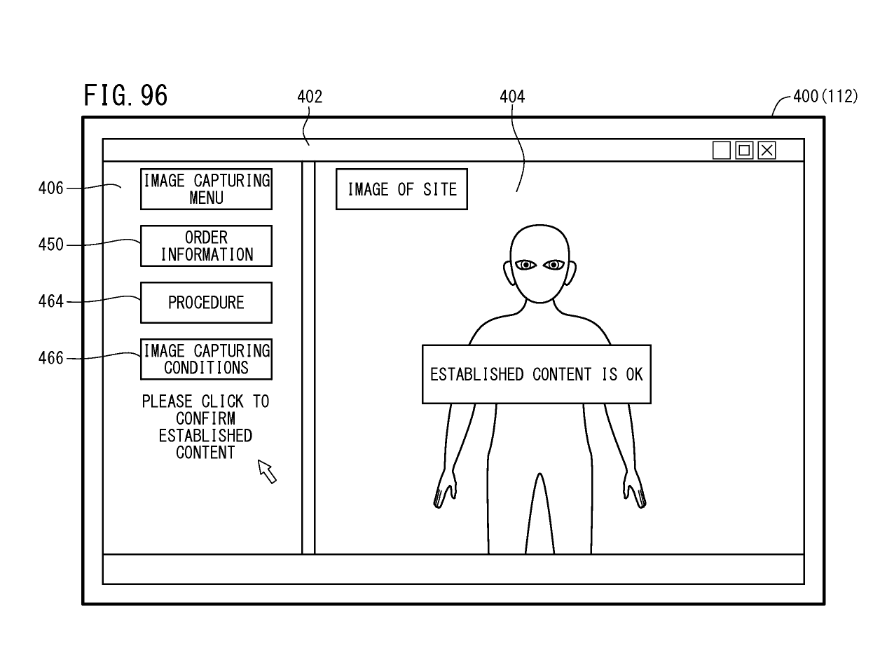

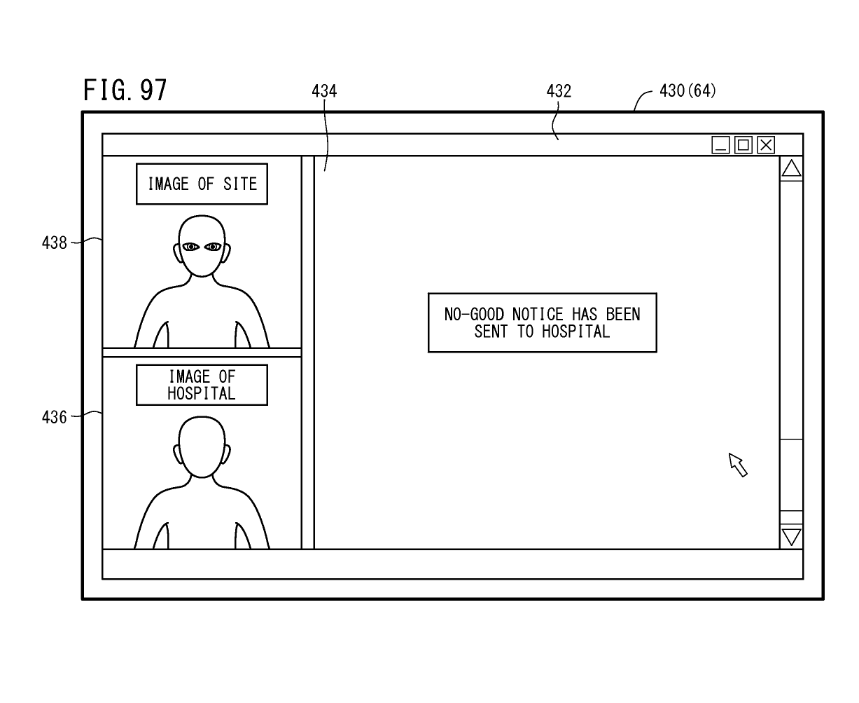

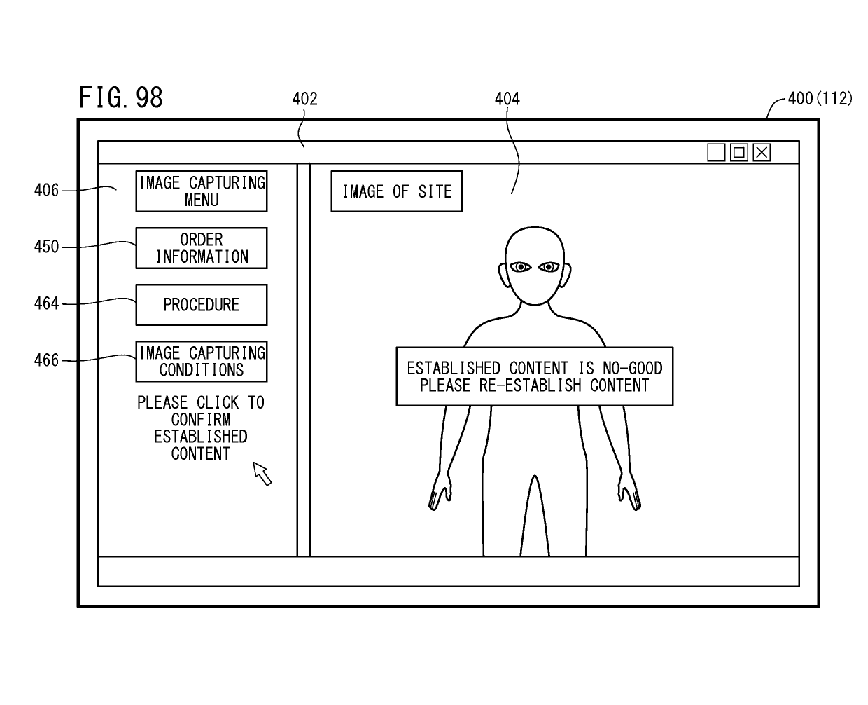

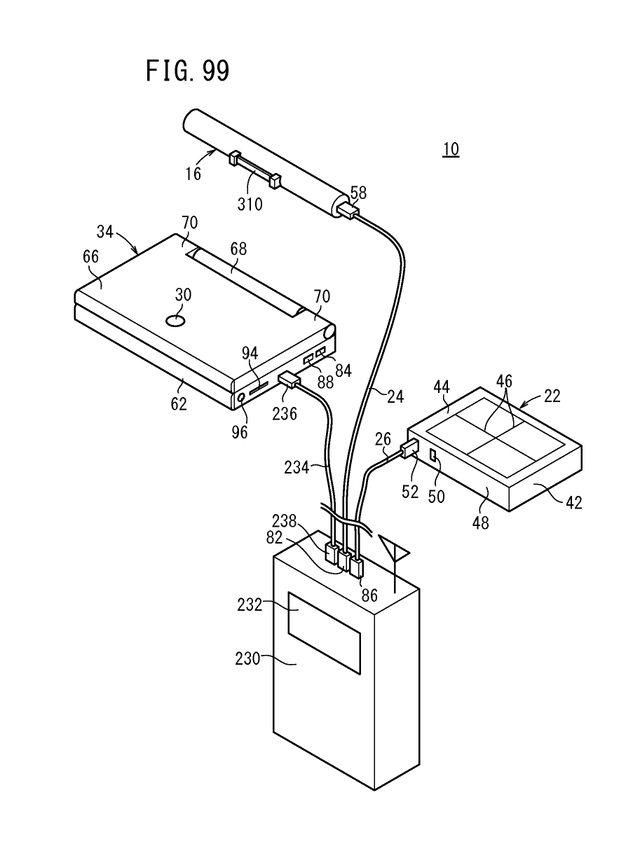

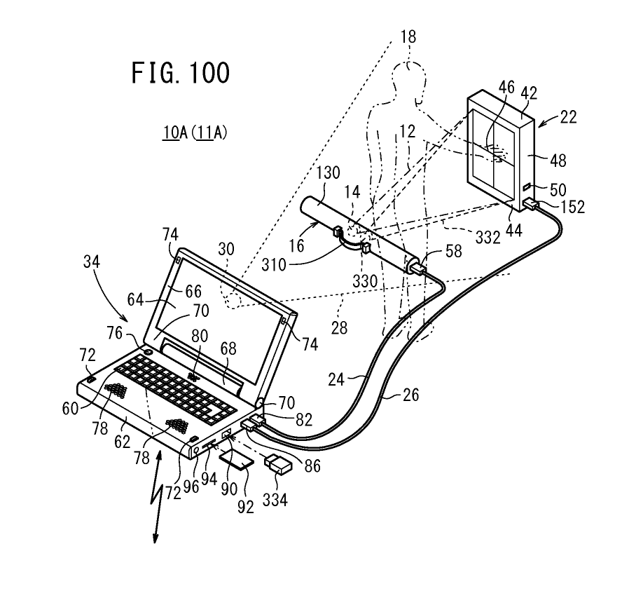

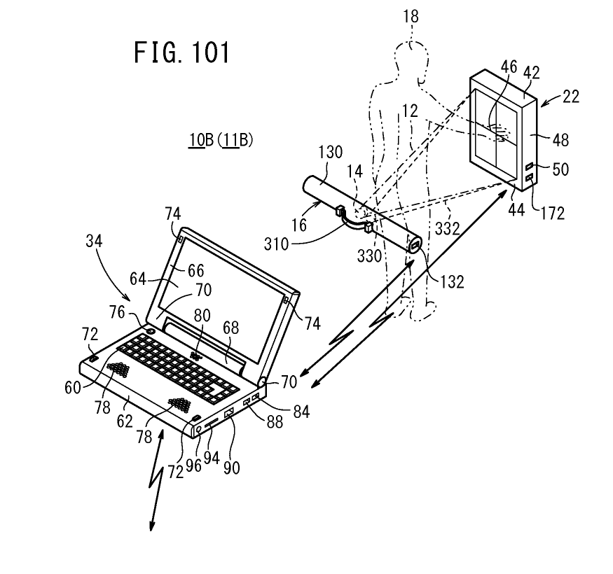

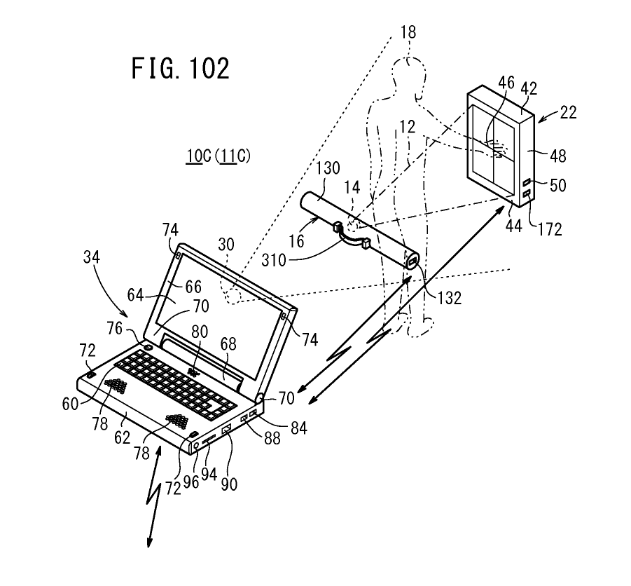

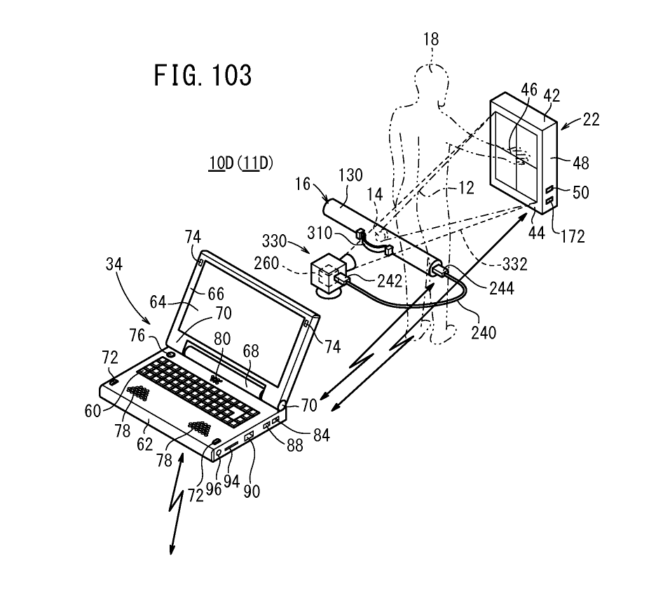

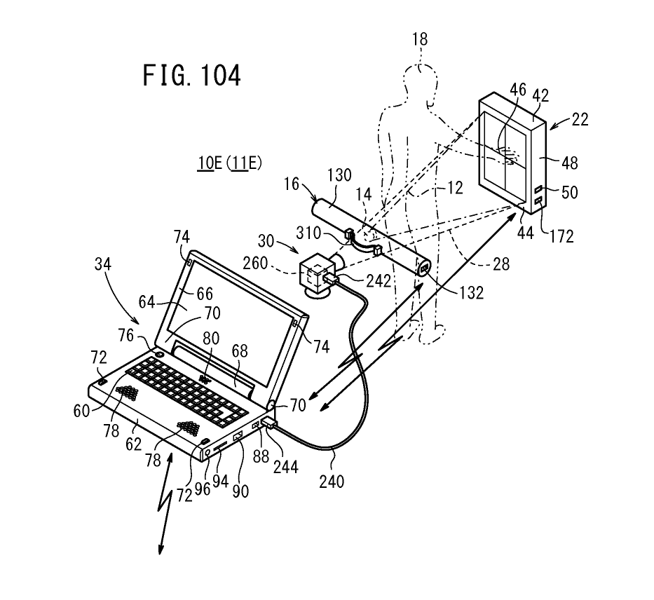

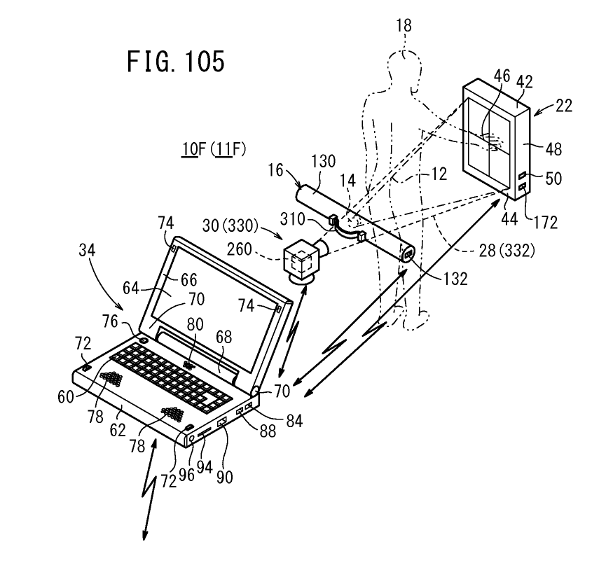

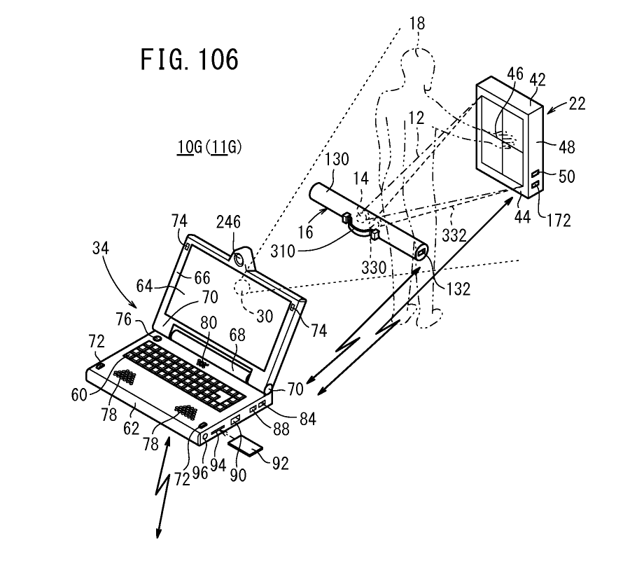

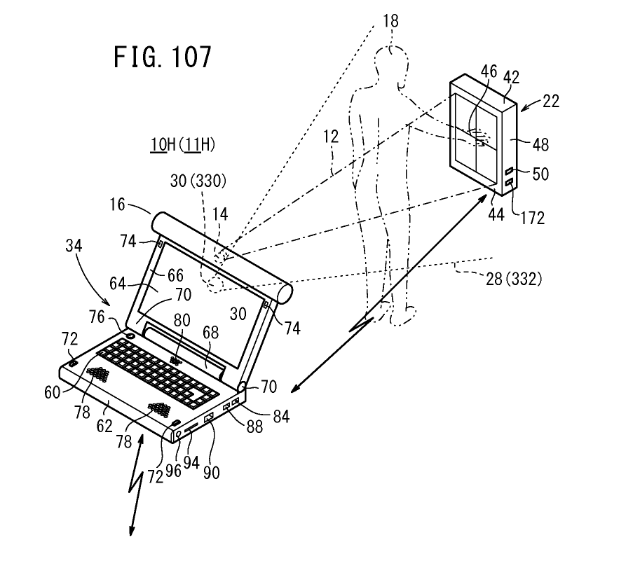

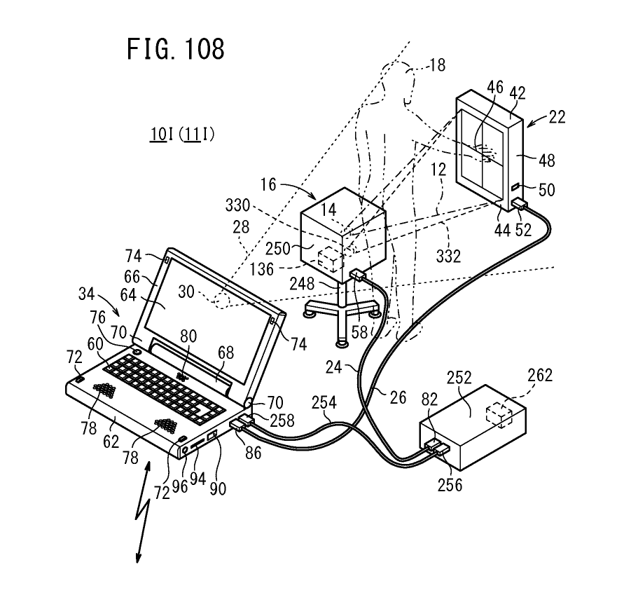

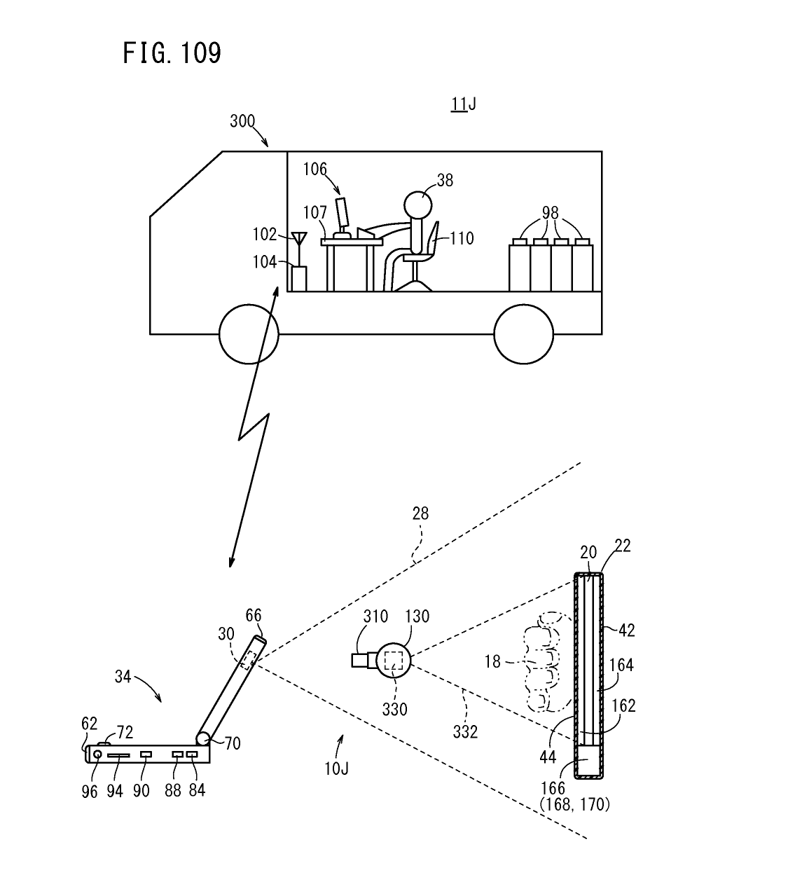

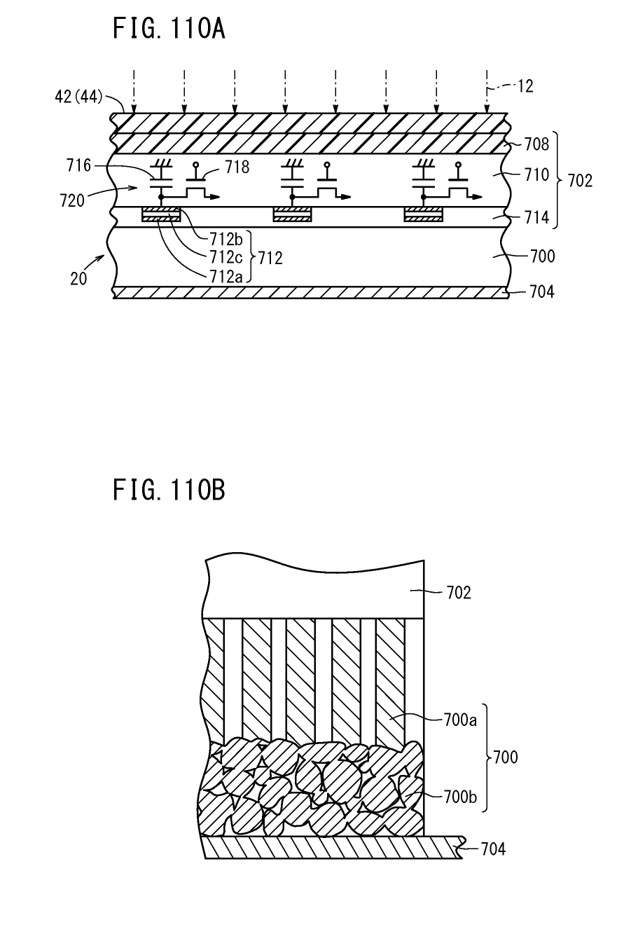

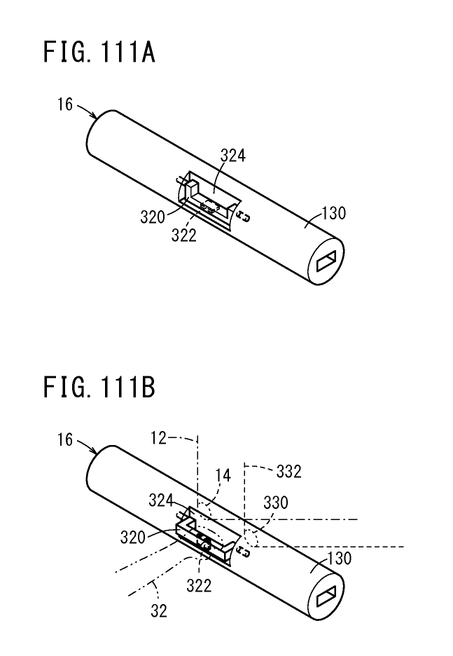

As shown in FIGS. 1, 2, 4 through 6, and 9, the cassette device 22 includes a substantially rectangular housing 42 made of a material permeable to radiation 12. The housing 42 includes a surface, referred to as an irradiated surface 44, which faces toward the radiation source device 16 and is irradiated with radiation 12. The cassette device 22 has crisscross guide lines 46 disposed within an irradiated area (irradiated field), which is irradiated with radiation 12, of the irradiated surface 44, and which serves as a reference for an image capturing area and an image capturing position. The guide lines 46 provide an outer frame (irradiated field), which as shown in FIG. 9, is substantially aligned with the outer edge of the radiation detector 20 as viewed in plan. One side 48 of the housing 42 supports a switch 50 for activating the cassette device 22, and a USB terminal 172 for connection to a USB cable, not shown.

As shown in FIGS. 1 through 6, the radiation source device 16 has a substantially cylindrical casing 130 made of a material permeable to radiation 12. The radiation source device 16 includes a handle 310 to be gripped by the operator 32, disposed on a side of the casing 130 diametrically opposite from the side on which radiation 12 is output. The web camera 330 is disposed in the radiation source device 16 in the vicinity of the side from which radiation 12 is output. The operator 32 can grip the handle 310 with one hand, so as to direct the radiation source device 16 incorporating the web camera 330 therein toward the cassette device 22. At the same time, the operator 32 can operate the portable information terminal 34 with the other hand.

In this manner, by directing the radiation source device 16 toward the cassette device 22, the operator 32 can apply radiation 12 from the radiation source 14 to the cassette device 22, and can capture a camera image (radiation source optical image, site optical image) of the cassette device 22 with the web camera 330.

The web camera 330 captures an image of a given region, which includes a region (a right hand of the subject 18 in FIGS. 1 and 2) to be imaged of the subject 18, and a range including the guide lines 46 of the cassette device 22, which serves as the predetermined imaging area 332, while the subject 18 is situated between the radiation source device 16 and the cassette device 22. Alternatively, the web camera 330 can capture an image of the predetermined imaging area 332, such that the face of the subject 18 including the subject's pupils is included within the predetermined imaging area 332.

The handle 310 includes an electrostatic capacitance or a resistance film type of touch sensor 312. If the operator 32 grips the handle 310, the hand of the operator 32 contacts an electrode, not shown, of the touch sensor 312, which then outputs a detection signal based on contact between the operator's hand and the electrode.

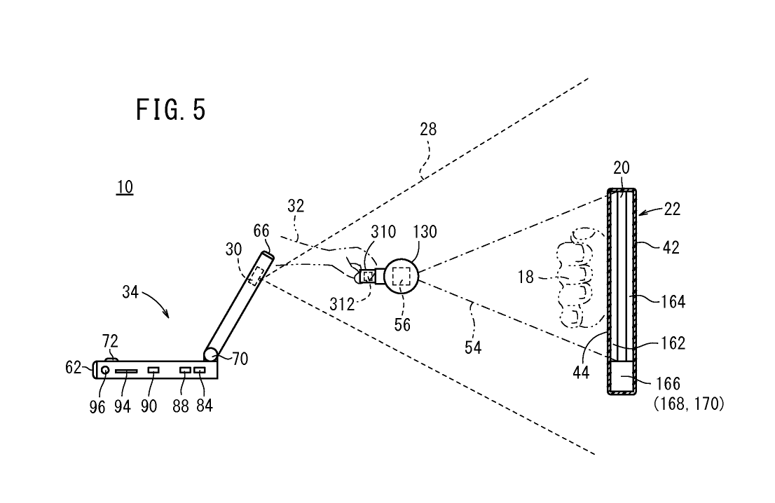

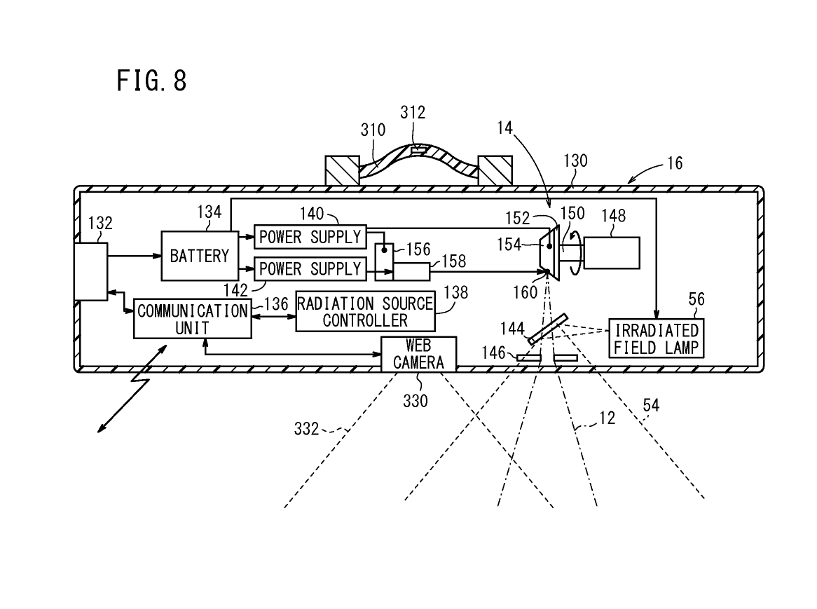

The radiation source device 16 houses therein, in addition to the radiation source 14 and the web camera 330, an irradiated field lamp 56 for emitting irradiation light 54 (see FIGS. 5 and 8). The irradiated field lamp 56 applies irradiation light 54 to the irradiated surface 44 before the radiation source 14 outputs radiation 12, thereby illuminating the irradiated field on the irradiated surface 44. A USB terminal 132 for connection to a USB cable, not shown, is disposed on one end of the casing 130.

It is assumed that a straight line interconnecting a focus point 160, to be described later, of the radiation source 14 and a central position of the guide lines 46, i.e., a point of intersection between the criss-crossing guide lines 46, is substantially perpendicular to the irradiated surface 44. If the distance (imaging distance) between the focus point 160 and the central position of the crisscross guide lines 46 is set to a source-to-image distance (SID), then the outer edge of the irradiated field, which is displayed on the irradiated surface 44 upon application of irradiation light 54 thereto, essentially is aligned with the outer frame of the guide lines 46. The portion of the casing 130 through which the irradiation light 54 passes preferably is made of a material permeable to the irradiation light 54, for example.

As shown in FIGS. 1, 2, 4 through 7, and 99, the portable information terminal 34 comprises a notebook-sized personal computer including an operating unit 60 such as a keyboard, etc., disposed on an upper surface (facing a lid 66) of a main body 62, and a display unit 64 (portable terminal display unit, indicating unit) disposed on a lower surface (facing the operating unit 60) of the lid 66.

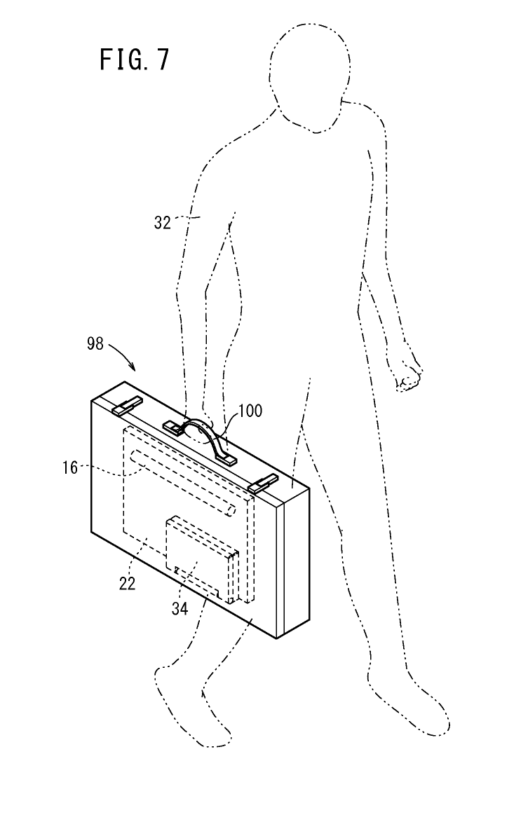

During times that the portable information terminal 34 is not in use, the main body 62 and the lid 66 are folded over one another about a shaft 68 on one side of the main body 62 and two hinges 70 connected to respective ends of the shaft 68, as shown in FIGS. 7 and 99. The upper surface of the main body 62 has two teeth 72, and the lower surface of the lid 66 has two recesses 74 corresponding respectively to the two teeth 72. If the upper surface of the main body 62 and the lower surface of the lid 66 are brought into contact with each other at a time that the portable information terminal 34 is not in use, the teeth 72 are fitted respectively into the recesses 74, thereby keeping the main body 62 and the lid 66 folded over each other.

During times that the portable information terminal 34 is in use, the lid 66 is turned away from the main body 62 about the shaft 68 and the hinges 70, thereby unfolding the main body 62 and the lid 66 away from each other, from the folded position shown in FIGS. 7 and 99 to the operational position shown in FIGS. 1, 2, and 4 through 6.

On the upper surface of the main body 62 in the vicinity of the operating unit 60, there are provided a power supply switch 76 for activating the portable information terminal 34, speakers 78 for outputting speech sounds, and a microphone 80 for detecting voices of the subject 18 and the operator 32.

On one side of the main body 62, there are provided USB terminals 84, 88, 90 for connecting USB cables, not shown, and a USB memory 334, a card slot 94 for insertion of a memory card 92, and an input terminal 96 for connection to an AC adapter.

The aforementioned web camera 30 is disposed on the upper surface of the lid 66. Assuming that the lid 66 is turned away from the main body 62 and that the operator 32 has turned on the power supply switch 76 to activate the portable information terminal 34, the web camera 30 captures a camera image (portable terminal optical image, on-site optical image) of the imaging area 28. More preferably, the web camera 3 captures an image of the predetermined imaging area 332, such that the face of the subject 18, including the subject's pupils, is included within the predetermined imaging area 332, or captures an image of a given region, which includes the radiation source device 16, a region to be imaged of the subject 18, and a range including the guide lines 46 of the cassette device 22, which serves as the predetermined imaging area 332, while the subject 18 is situated between the radiation source device 16 and the cassette device 22.

A notebook-sized portable information terminal 34 according to the present exemplary embodiment will be described below. However, the portable information terminal 34 may be a portable terminal having various functions, including the operating unit 60, the display unit 64, etc., such as a hand-held personal computer (tablet PC) incorporating a touch panel display unit, a mobile phone with a screen display function, or a PDA (personal information terminal).

FIG. 7 shows a manner in which the radiographic image capturing apparatus 10 is carried by the operator 32.

During times that the radiographic image capturing apparatus 10 is carried by the operator 32, the radiation source device 16, the cassette device 22, and the folded portable information terminal 34 are housed in an attaché case 98. The operator 32 can grip the grip 100 and carry the attaché case 98 from the medical organization 40 (see FIGS. 1 and 13) to a desired location, e.g., a disaster site or a home care treatment site. At the location where the attaché case 98 is carried, the operator 32 can remove the radiation source device 16, the cassette device 22, and the folded portable information terminal 34 from the attaché case 98, and assemble these components into the configuration shown in FIGS. 1 through 6. The operator 32 can then perform a preparatory procedure in order to ready the radiographic image capturing apparatus 10 for capturing radiographic images of a disaster victim at a disaster site or a home-care-service recipient at a home care treatment site.

The radiographic image capturing apparatus 10 according to the present exemplary embodiment can be referred to as a portable radiographic image capturing apparatus, in which the web camera 30 and the portable information terminal 34 are integral with each other, and further in which the web camera 330 and the radiation source device 16 are integral with each other. The disaster victim or the home-care-service recipient, who is a subject to be imaged in order to produce a radiographic image thereof, will be referred to as the subject 18.

As shown in FIG. 1, the medical organization 40 includes a communication unit (waiting location communication unit) 104 having an antenna 102 for sending signals to and receiving signals from the portable information terminal 34 via the network 36 by way of wireless communications. A console 106 is electrically connected to the communication unit 104.

The console 106 is connected to a radiology information system (RIS), not shown, which generally manages radiographic images and other information that are handled in a radiological department of the medical organization 40. The RIS is connected to a hospital information system (HIS), not shown, which generally manages medical information in the medical organization 40.

The console 106 is placed on a desk 107 in a room where a doctor 38 in the medical organization 40 is present.

The console 106 comprises a main body 108 for carrying out various processing sequences, a display unit 112 (console display unit, indicating unit, warning unit), an operating unit 114 such as a keyboard, etc., operable by the doctor 38 and a mouse 336, a web camera 116 (waiting location camera), a speaker 118 (indicating unit, warning unit) for outputting speech sounds, an exposure switch 120, and a microphone 122 for detecting the voice of the doctor 38.

The display unit 112 is a display for displaying various images and information for the doctor 38, who is seated in a chair 110 at the desk 107. The web camera 116 is mounted on an upper end of the display unit 112 for capturing an image of the doctor 38. The exposure switch 120 can be turned on by the doctor 38 in order to instruct the radiation source 14 to emit radiation 12.

As described above, the portable information terminal 34 and the communication unit 104 send signals to and receive signals from each other via the network 36 by way of wireless communications. The portable information terminal 34, the radiation source device 16, and the cassette device 22 also send signals to and receive signals from each other by way of wireless communications.

The portable information terminal 34 is capable of sending camera images output from the web cameras 30 and 330, a radiographic image supplied from the cassette device 22 (radiation detector 20), and voice signals representative of voices of the operator 32 or the subject 18 detected by the microphone 80, through the antenna 102 of the medical organization 40 to the communication unit 104 via the network 36 by way of wireless communications.

The communication unit 104 is capable of sending a camera image (a waiting location optical image such as a moving image, still images captured intermittently, or still images captured at predetermined times) of the doctor 38 captured by the web camera 116, an exposure control signal generated in the main body 108 based on turning-on of the exposure switch 120 by the doctor 38, and a voice signal representing the voice of the doctor 38 detected by the microphone 122, to the portable information terminal 34 via the antenna 102 and the network 36 by way of wireless communications. The communication unit 104 also sends an image capturing menu concerning capturing of the radiographic image, which includes order information, procedures, and image capturing conditions, to the portable information terminal 34 via the antenna 102 and the network 36 by way of wireless communications.

On the portable information terminal 34, the display unit 64 is capable of displaying at least one of a camera image of the imaging area 28 captured by the web camera 30, a camera image of the imaging area 332 captured by the web camera 330, a radiographic image from the radiation detector 20, and a camera image of the doctor 38 captured by the web camera 116. The display unit 64 also is capable of displaying information (character information) corresponding to the voice and exposure control signals referred to above, and the content of the image capturing menu. The speakers 78 are capable of outputting the voice of the doctor 38, and a sound that depends on the exposure control signal (an alarm sound indicative of the start of emission of radiation 12 from the radiation source 14).

The portable information terminal 34 sends a synchronization control signal, which is generated based on the exposure control signal, to the radiation source device 16 and the cassette device 22, and controls the radiation source 14 and the cassette device 22 according to such order information, procedures, and the image capturing conditions of the image capturing menu, for thereby capturing a radiographic image of the subject 18 while synchronizing start of emission of radiation 12 from the radiation source 14 and detection and conversion of radiation 12 into a radiographic image in the radiation detector 20 with each other.

On the console 106, the display unit 112 is capable of displaying at least one of a camera image in the imaging area 28 captured by the web camera 30, a camera image of the imaging area 332 captured by the web camera 330, a radiographic image from the radiation detector 20, a camera image of the doctor 38 captured by the web camera 116, a camera image of the doctor 38 captured by the web camera 116, and an image capturing menu, similar to the case of the display unit 64. The display unit 112 also is capable of displaying information (character information) corresponding to the voice and exposure control signals referred to above. The speaker 118 is capable of outputting voices of the operator 32 or the subject 18 and sounds depending on the exposure control signal.

The order information of the image capturing menu is made up of information, which is generated by the doctor 38 using the RIS, for instructing the radiographic image capturing apparatus 10 to capture a radiographic image of the subject 18. More specifically, the order information includes subject information for identifying the subject 18, including the name, age, gender, etc., of the subject 18 (patient), information concerning a method of capturing a radiographic image, e.g., a simple image capturing method, capturing of an image of a hand, etc. The procedures represent information concerning a region to be imaged of the subject 18, and a direction along which radiation 12 is to be applied to the region to be imaged. The image capturing conditions refer to various conditions under which the region to be imaged of the subject 18 is irradiated with radiation 12, including a tube voltage and a tube current of the radiation source 14, an irradiation time of the radiation 12, etc.

The doctor 38 is able to rewrite (set or change) the content of the image capturing menu including the present order information into content that depends on the subject 18 included in the camera image, while viewing the camera images (optical images of the subject 18) captured by the web cameras 30, 330 and displayed on the display unit 112. The rewritten image capturing menu is sent to the portable information terminal 34, thereby enabling the portable information terminal 34 to capture a radiographic image of the subject 18 while controlling the radiation source device 16 and the cassette device 22 based on the rewritten image capturing menu.

Internal structural details of the radiation source device 16 and the cassette device 22 will be described in specific detail below with reference to FIGS. 8 through 11.

As shown in FIG. 8, the casing 130 of the radiation source device 16 houses therein the radiation source 14, the irradiated field lamp 56, the web camera 330, the USB terminal 132, a battery 134, a communication unit 136 (on-site communication unit), a radiation source controller 138 for controlling the radiation source 14, a mirror 144 made of a material permeable to radiation 12, and a collimator 146, which is made of a material impermeable to radiation 12 but is permeable to the irradiation light 54. The battery 134 can be charged from an external source via the USB terminal 132, and is capable of supplying electric power to various components of the radiation source device 16.

The radiation source 14 comprises a field-emission-type radiation source.

More specifically, the radiation source 14 includes a disk-shaped rotary anode 152 mounted on a rotational shaft 150, which can be rotated about its axis through a rotating mechanism 148, an annular target layer 154 disposed on the surface of the rotary anode 152 and which is made mainly of a metallic element such as Mo or the like, a cathode 156 disposed in confronting relation to the rotary anode 152, and a field-emission-type electron source 158 disposed on the cathode 156 in confronting relation to the target layer 154.

The radiation source controller 138 controls the radiation source 14 in order to output radiation 12 at a prescribed dose according to a synchronization control signal based on an exposure control signal, which is received from the portable information terminal 34 (see FIGS. 1, 2, and 4 through 7) via the communication unit 136 by way of wireless communications, and according to the image capturing conditions of the image capturing menu.

More specifically, the radiation source 14 is controlled by the radiation source controller 138 to output radiation 12 in the following manner. The rotating mechanism 148 rotates the rotational shaft 150 to thereby rotate the rotary anode 152. The battery 134 supplies electric power to a power supply 142, which applies a voltage (negative voltage) to the field-emission-type electron source 158. The battery 134 also supplies electric power to a power supply 140, which applies a voltage between the rotary anode 152 and the cathode 156, i.e., the power supply 140 applies a positive voltage to the rotary anode 152 and a negative voltage to the cathode 156.

The field-emission-type electron source 158 emits electrons, which are accelerated to bombard the target layer 154 under the voltage applied between the rotary anode 152 and the cathode 156. The bombarded surface (focus point 160) of the target layer 154 emits radiation 12 at an intensity level depending on the applied electrons. Radiation 12 passes through the mirror 144, the irradiation area thereof is constricted by the collimator 146, and radiation 12 is output from the radiation source device 16.

Until the radiation source controller 138 is supplied with the synchronization control signal from the portable information terminal 34, the radiation source controller 138 controls the irradiated field lamp 56 in order to emit irradiation light 54. The irradiation light 54 emitted from the irradiated field lamp 56 is reflected by the mirror 144 in the direction of the collimator 146, and is output from the radiation source device 16.

The handle 310 incorporates the touch sensor 312 therein. As described above, after the operator 32 has gripped the handle 310, the radiation source 14 applies radiation 12 toward the subject 18 and the cassette device 22. Therefore, at the time that the touch sensor 312 outputs a detection signal, the radiation source controller 138 starts to supply electric power from the battery 134 to various components of the radiation source device 16, thereby activating the radiation source device 16.

As shown in FIGS. 4 through 6 and 9, the cassette device 22 houses therein a grid 162 for removing scattered rays of radiation 12 from the subject 18 if the radiation source 14 applies radiation 12 to the subject 18, the radiation detector 20, and a lead plate 164 for absorbing back scattered rays of radiation 12, which are successively arranged in this order from the irradiated surface 44 of the cassette device 22, which faces toward the subject 18. The irradiated surface 44 of the cassette device 22 may be constructed as the grid 162.

The radiation detector 20 may comprise an indirect conversion type of radiation detector including a scintillator for converting radiation 12 having passed through the subject 18 into visible light, and solid-state detectors (hereinafter also referred to as pixels) made of amorphous silicon (a-Si) or the like for converting visible light into electric signals, or a direct conversion type of radiation detector comprising solid-state detectors made of amorphous selenium (a-Se) or the like for converting the dose of radiation 12 directly into electric signals.

The switch 50 and a USB terminal 172 are disposed on the side 48 of the cassette device 22.

The cassette device 22 also houses therein a battery 166, a cassette controller 168, and a communication unit 170 (on-site communication unit).

The battery 166, which is chargeable from an external device through the USB terminal 172, supplies electric power to various components (the radiation detector 20, the cassette controller 168, the communication unit 170) of the cassette device 22. The cassette controller 168 controls the radiation detector 20 with electric power supplied from the battery 166. The communication unit 170 sends and receives signals, including information of the radiation 12 detected by the radiation detector 20, to and from the portable information terminal 34 by way of wireless communications.

A plate of lead or the like preferably is placed over the side surfaces of the cassette controller 168 and the communication unit 170 under the irradiated surface 44, so as to protect the cassette controller 168 and the communication unit 170 against damage, which otherwise would be caused if these components were irradiated with radiation 12.

As described above, if the operator 32 grips the handle 310, the touch sensor 312 outputs a detection signal, and thereafter, the radiation source 14 outputs radiation 12. Therefore, the radiation source device 16 directly sends the detection signal to the cassette device 22 by way of wireless communications, or sends the detection signal via the portable information terminal 34 to the cassette device 22 by way of wireless communications. Upon receiving the detection signal via the communication unit 170, the cassette controller 168 can control the battery 166 to start supplying electric power to various components of the cassette device 22.

As schematically shown in FIG. 10, the radiation detector 20 comprises a number of pixels 180 arrayed on a substrate, not shown, a number of gate lines 182 for supplying control signals to the pixels 180, and a number of signal lines 184 for reading electric signals output from the pixels 180.

A circuit arrangement of the cassette device 22, which incorporates an indirect conversion type of radiation detector 20, for example, will be described in detail below with reference to FIG. 11.

As shown in FIG. 11, the radiation detector 20 comprises an array of TFTs 188 arranged in rows and columns, and a photoelectric conversion layer 186 including pixels 180 and made of a material such as amorphous silicon (a-Si) or the like for converting visible light into electric signals. The photoelectric conversion layer 186 is disposed on the array of TFTs 188. If radiation 12 is applied to the radiation detector 20, the pixels 180, which are supplied with a bias voltage Vb from the battery 166, generate electric charges by converting visible light into analog electric signals. The TFTs 188 are turned on along each row at a time, whereupon the electric charges are read from the pixels 180 as an image signal.

The TFTs 188 are connected to the respective pixels 180. The gate lines 182, which extend parallel to the rows, and the signal lines 184, which extend parallel to the columns, are connected to the TFTs 188. The gate lines 182 are connected to a line scanning driver 190, and the signal lines 184 are connected to a multiplexer 192. The gate lines 182 are supplied with control signals Von, Voff from the line scanning driver 190 for turning on and off the TFTs 188 along the rows. The line scanning driver 190 comprises a plurality of switches SW1 for switching between the gate lines 182, and an address decoder 194 for outputting a selection signal for selecting one of the switches SW1 at a time. The address decoder 194 is supplied with an address signal from the cassette controller 168.

The signal lines 184 are supplied with electric charges that are stored by the pixels 180 through the TFTs 188 arranged in columns. Electric charges supplied to the signal lines 184 are amplified by amplifiers 196, which are connected respectively to the signal lines 184. The amplifiers 196 are connected to the multiplexer 192 through respective sample and hold circuits 198. The multiplexer 192 comprises a plurality of switches SW2 for successively switching between the signal lines 184, and an address decoder 200 for outputting selection signals for selecting one of the switches SW2 at a time. The address decoder 200 is supplied with address signals from the cassette controller 168. The multiplexer 192 has an output terminal connected to an A/D converter 202. A radiographic image signal, which is generated by the multiplexer 192 based on electric charges from the sample and hold circuits 198, is converted by the A/D converter 202 into a digital image signal representing radiographic image information, which is supplied to the cassette controller 168.

The TFTs 188, which function as switching devices, may be combined with another image capturing device such as a CMOS (Complementary Metal-Oxide Semiconductor) image sensor or the like. Alternatively, the TFTs 188 may be replaced with a CCD (Charge-Coupled Device) image sensor for shifting and transferring electric charges by way of shift pulses, which correspond to gate signals in the TFTs.

FIG. 12 shows in block form the radiographic image capturing apparatus 10, and FIG. 11 shows in block form the medical organization 40. Components of the radiographic image capturing apparatus 10 and the medical organization 40, which have not been described above with reference to FIGS. 1 through 11, will be described below with reference to FIGS. 12 and 13.

The cassette controller 168 of the cassette device 22 comprises an image memory 210, an address signal generator 212, and a cassette ID memory 214.

The address signal generator 212 supplies address signals to the address decoder 194 of the line scanning driver 190, and to the address decoder 200 of the multiplexer 192. The image memory 210 stores radiographic images detected by the radiation detector 20. The cassette ID memory 214 stores cassette ID information for identifying (the cassette device 22 of) the radiographic image capturing apparatus 10.

The cassette controller 168 sends the cassette ID information stored in the cassette ID memory 214, and the radiographic image information stored in the image memory 210 to the portable information terminal 34 by way of wireless communications via the communication unit 170 and the antenna 342.

The radiation source device 16 sends signals to and receives signals from the portable information terminal 34 by way of wireless communications via the communication unit 136 and the antenna 340.

The portable information terminal 34 further includes a communication unit 218 (on-site communication unit), a battery 220 for supplying electric power to various components of the portable information terminal 34, a control processor (judgment processor) 222 for performing various control processes, and a memory 224 for storing camera images, radiographic images, etc.

The communication unit 218 sends signals to and receives signals from an external circuit by way of wireless communications via an antenna 216, sends signals to and receives signals from external circuits by way of wired communications via the USB terminals 84, 88, 90, sends signals to and receives signals from the USB memory 334 (see FIG. 1) via the USB terminals 84, 88, 90, and sends signals to and receives signals from the memory card 92, which is inserted in the card slot 94.

If the operator 32 turns on the power supply switch 76, the battery 220 supplies electric power to the web camera 30, the speakers 78, the microphone 80, the communication unit 218, and the control processor 222. While the portable information terminal 34 is electrically connected by USB cables to the radiation source device 16 and the cassette device 22, the battery 220 is capable of charging the batteries 134, 166 via the USB cables. The battery 220 can also be charged from an external circuit via the input terminal 96 or a USB cable.

With the portable information terminal 34 electrically connected by USB cables to the radiation source device 16 and the cassette device 22, electric power can be supplied and signals can be sent and received (transmitted) between the portable information terminal 34, the radiation source device 16, and the cassette device 22 by way of wired communications. Unless otherwise noted, according to the present exemplary embodiment, it is assumed that signals are sent and received between the portable information terminal 34, the radiation source device 16, and the cassette device 22 by way of wireless communications.

The control processor 222 comprises a CPU of the portable information terminal 34, which carries out various control sequences by reading and executing programs stored in the memory 224.

More specifically, the control processor 222 stores in the memory 224 camera images captured by the web cameras 30, 330 together with radiographic images and cassette ID information received from the cassette device 22 via the communication unit 218. The control processor 222 also controls the display unit 64 to display at least one of the camera images, the radiographic images, and the image capturing menu, and sends at least one of the camera images captured by the web cameras 30, 330, and the radiographic images and the cassette ID information by way of wireless communications to the medical organization 40 via the communication unit 218, the antenna 216, and the network 36. The control processor 222 also sends a voice signal representing the voice of the operator 32 or the voice of the subject 18 detected by the microphone 80 by way of wireless communications to the medical organization 40 via the communication unit 218, the antenna 216, and the network 36.

The control processor 222 controls the display unit 64 to display a camera image (of the doctor 38) captured by the web camera 116 and received from the medical organization 40 via the network 36, the antenna 216, and the communication unit 218, and outputs the voice of the doctor 38 from the speakers 78 based on the voice signal received from the medical organization 40. If the control processor 222 receives an exposure control signal from the medical organization 40, the control processor 222 generates a synchronization control signal depending on the received exposure control signal, and sends the generated synchronization control signal and the image capturing conditions of the image capturing menu to the radiation source device 16 and the cassette device 22, to thereby capture a radiographic image while synchronizing the output of radiation 12 from the radiation source 14 and the detection and conversion by the radiation detector 20 of the radiation 12 into the radiation image information.

The display unit 64 is capable of displaying the image capturing menu received by the control processor 222 and the camera image of (the face, including the pupils of) the subject 18 captured by the web camera 30 or the web camera 330. Therefore, the operator 32 can judge whether or not the subject 18 registered in the order information of the image capturing menu and the subject 18 included in the camera image are in agreement with each other.

More specifically, the operator 32 operates the operating unit 60 while viewing the content displayed on the display unit 64, and enters content indicative of a judgment result (a result indicating whether or not the subjects 18 are in agreement with each other). If the entered content indicates that the subjects 18 are in agreement with each other, then the control processor 222 sends a result (judgment result), which indicates that a radiographic image can be captured according to the image capturing menu, by way of wireless communications to the medical organization 40 via the communication unit 218, the antenna 216, and the network 36. If the entered content indicates that the subjects 18 are not in agreement with each other, then since a radiographic image cannot be captured according to the image capturing menu, the control processor 222 sends a result (judgment result), in order to request that the image capturing menu be rewritten in an image capturing unit depending on the subject 18 included in the camera image, by way of wireless communications to the medical organization 40 via the communication unit 218, the antenna 216, and the network 36.

The console 106 also includes a memory 228 and a control processor 226 for performing various control sequences. The control processor 226 includes an authentication processor 344, a control signal generator 346, and an association processor 348.

The control processor 226 comprises a CPU of the main body 108, which carries out various control sequences by reading and executing programs stored in the memory 228.

More specifically, the control processor 226 sends a camera image captured by the web camera 116 to the portable information terminal 34 by way of wireless communications via the communication unit 104, the antenna 102, and the network 36. The control processor 226 also sends a voice signal representing the voice of the doctor 38 detected by the microphone 122 to the portable information terminal 34 by way of wireless communications via the communication unit 104, the antenna 102, and the network 36. The control processor 226 further outputs a voice signal sent from the portable information terminal 34 by way of wireless communications from the speaker 118, the voice signal representing the voice of the operator 32 or the subject 18. The control processor 226 may display the camera image, which is captured by the web camera 116, on the display unit 112.

If the operator 32 finds a subject 18 according to the order information of the image capturing menu displayed on the display unit 64 of the portable information terminal 34, and the web camera 30 or 330 captures a camera image (e.g., a still image) of the face including the pupils of the subject 18, then the portable information terminal 34 sends the camera image by way of wireless communications via the network 36, the antenna 102, and the communication unit 104. The authentication processor 344 performs a biological authentication process using the pupils or face of the subject 18 that is included in the camera image, or a pupil authentication process using the pupils of the subject 18, on the camera image received via the network 36, the antenna 102, and the communication unit 104, thereby judging whether or not the subject 18 having the pupils and the subject 18 registered in the order information are in agreement with each other.

If the subjects 18 are in agreement with each other, then the authentication processor 344 determines that the subject 18 included in the camera image is a true subject 18 according to the image capturing menu, and sends an authentication result, which indicates that a radiographic image of the subject 18 can be captured, to the portable information terminal 34 by way of wireless communications. If the subjects 18 are not in agreement with each other, then the authentication processor 344 determines that the operator 32 has found a subject 18 that differs from the subject 18 indicated by the image capturing menu, and sends an authentication result, which instructs the operator 32 to find the true subject 18 according to the image capturing menu, to the portable information terminal 34 by way of wireless communications.

If the doctor 38 turns on the exposure switch 120, the control signal generator 346 generates an exposure control signal for initiating emission of radiation 12 from the radiation source 14, and sends the generated exposure control signal to the portable information terminal 34 via the communication unit 104, the antenna 102, and the network 36 by way of wireless communications.

If a region to be imaged of the subject 18 is not visible, or if only a portion of the region is included within the outer frame of the guide lines 46 on a camera image captured by the web camera 30 and displayed on the display unit 112, then the doctor 38 judges that an appropriate radiographic image of the region to be imaged of the subject 18 cannot be acquired by applying radiation 12 to the subject 18. The doctor 38 does not turn on the exposure switch 120, but gives a voice instruction via the microphone 122 to change the position or attitude of the subject 18 until the region to be imaged is included within the outer frame of the guide lines 46. If the exposure switch 120 is not turned on, therefore, the control signal generator 346 does not generate an exposure control signal.

After a radiographic image has been captured, the control processor 226 associates the camera images captured by the web cameras 30 and 330, the radiographic image, and the cassette ID information, which is sent from the portable information terminal 34, with the image capturing menu, and stores these items in the memory 228. Since, as described above, the web camera 116 has captured a camera image of the doctor 38, the camera image of the doctor 38 also may be associated with the above items of information that are stored in the memory 228.

Since the portable information terminal 34 includes the memory 224, the control processor 222 can function as an association processor. The control processor 222 can associate the image capturing menu, the camera images from the web cameras 30, 330, the radiographic image, and the cassette ID information with each other, and store these items in the memory 224, after which the associated items of information are sent from the portable information terminal 34 to the medical organization 40. In this case, the association processor 348 stores the received associated pieces of information in the memory 228.

The associated camera image, which is captured by the web camera 30, preferably is an image of the face (including the pupils) of the subject 18, which is captured by the web camera 30 before capturing of the radiographic image. The associated camera image, which is captured by the web camera 330, preferably is an image of the region to be captured of the subject 18, which is captured by the web camera 30 before a radiographic image is captured, and which is positioned with respect to the cassette device 22. For example, if the right hand of the subject is injured and a radiographic image is captured of the injured right hand as a region to be imaged, then the camera image captured by the web camera 330 should be an image that shows how the region to be image (right hand) is injured.

### [Operations of the Present Exemplary Embodiment]

The radiographic image capturing system 11 according to the present exemplary embodiment basically is constructed as described above. Operations (a radiographic image capturing method, an image displaying method) of the radiographic image capturing system 11 will be described below with reference to the flowcharts shown in FIGS. 14 through 22 and the display screens shown in FIGS. 23 through 98.

Characteristic functions (operations) of the present exemplary embodiment will be described below, as divided according to first through third embodiments.

The first embodiment is illustrated by way of the flowcharts shown in FIGS. 14 through 20 and the display screens shown in FIGS. 23 through 92. According to the first embodiment, the subject 18 to be imaged (see FIGS. 1, 2, 4 through 6, and 12) is identified in advance in the medical organization 40 (see FIGS. 1 and 13), and the medical organization 40 establishes in advance an image capturing menu, after which a radiographic image of the subject 18 is captured according to the established image capturing menu. The first embodiment is applicable, for example, to a situation in which the subject 18 is identified in advance at a home care treatment site, or the subject 18 is identified in advance in the medical organization 40, at a location where the doctor 38 is unable to attend to the subject 18 directly.

The second embodiment is illustrated in the flowchart shown in FIG. 21, and the display screen is shown in FIG. 93. According to the second embodiment, the doctor 38 at the medical organization 40 changes an image capturing menu while observing the subject 18, who is included in camera images captured by the web cameras 30 and 330 (see FIGS. 1, 2, 3B through 6, 8, and 12), and a radiographic image of the subject 18 is captured according to the changed image capturing menu. The second embodiment is applicable, for example, to a situation in which the operator 32 finds the subject 18 at a disaster site, and the doctor 38 rewrites an image capturing menu into an image capturing menu that depends on the subject 18, and captures a radiographic image of the subject 18 in an emergency.

The third embodiment is illustrated by way of the flowchart shown in FIG. 22 and the display screens shown in FIG. 94 through 98. According to the third embodiment, upon the image capturing menu rewritten according to the second embodiment being sent from the medical organization 40 to the portable information terminal 34 (see FIGS. 1, 2, 4 through 7, and 12), the site (operator 32) judges whether or not the region to be imaged of the subject 18 and the region to be imaged according to the image capturing menu are in agreement with each other, and the judgment result is indicated to the medical organization 40. The third embodiment is applicable, for example, to a situation in which the operator 32 encounters an injured hand and a radiographic image of the injured hand needs to be captured, but the image capturing menu indicates that a region other than the injured hand, such as a chest of the subject 18, should be imaged.

According to the first through third embodiments, it is assumed that the region to be imaged of the subject 18 is a right hand, and that a radiographic image of the right hand is to be captured. Stated otherwise, the right hand of the subject 18 is injured and a radiographic image of the right hand is captured. The first through third embodiments also will be described with reference to FIGS. 1 through 13 as necessary.

The first embodiment will initially be described below. In step S1 shown in FIG. 14, the operator 32 (see FIGS. 1, 2, and 3B through 7) carries the attaché case 98 from the medical organization 40, where the doctor 38 is unable to observe the subject 18 directly, to a disaster site, i.e., a home care treatment site, or a patient's bedroom in the medical organization 40, which cannot be attended by the doctor 38, according to directions from the doctor 38 who has legal authority to apply radiation 12 to the subject 18.

In step S2, after the operator 32 has arrived at the disaster site, the home care treatment site, or the patient's bedroom, the operator 32 removes the radiation source device 16, the cassette device 22, and the portable information terminal 34 from the attaché case 98. Then, the operator 32 lays out the portable information terminal 34, the radiation source device 16, and the cassette device 22 according to the positional relationship shown in FIGS. 1, 2, and 4 through 6.

In step S3, the operator 32 turns the lid 66 away from the main body 62 about the shaft 68 and the hinges 70, so as to unfold the portable information terminal 34 from the folded condition shown in FIGS. 7 and 99, and until the operating unit 60 and the display unit 64 are made visible, as shown in FIGS. 1, 2, and 4 through 6. Thereafter, the operator 32 turns on the power supply switch 76 in order to activate the portable information terminal 34.

If the power supply switch 76 is turned on, the battery 220 (see FIG. 12) begins supplying electric power to the web camera 30, the display unit 64, the microphone 80, the communication unit 218, and the control processor 222.

In this manner, upon activation of the portable information terminal 34, the control processor 222 sends ID information (e.g., cassette ID information) stored in the memory 224 to the medical organization 40 via the communication unit 218, the antenna 216, and the network 36 by way of wireless communications, thereby requesting the medical organization 40 to establish wireless communications between the medical organization 40 and the site while also authenticating the radiographic image capturing apparatus 10 in step S4.

In step S5, the control processor 226 (see FIG. 13) of the medical organization (waiting location) receives the ID information via the antenna 102 and the communication unit 104.

In step S6, the authentication processor 344 of the control processor 226 performs an authentication process on the ID information.

The authentication processor 344 judges whether or not the received ID information and the ID information (e.g., the cassette ID information) stored in the memory 228 are in agreement with each other, and in step S7, sends an authentication completion notice, which indicates a judgment result and also indicates that wireless communications have been established, to the portable information terminal 34 via the communication unit 104, the antenna 102, and the network 36. Thereafter, in step S8, the control processor 226 acquires order information from the RIS generated by the doctor 38 using the RIS.

In step S9, the control processor 222 of the portable information terminal 34 receives the authentication completion notice. The control processor 222 then determines that the ID information has been authenticated, and that wireless communications have been established between the medical organization 40 and the site. In step S10, the control processor 222 controls the web camera 30 to capture a camera image of the site, and then in step S11, starts to send the camera image captured by the web camera 30 to the medical organization 40.

If the camera image captured by the web camera 30 is a moving image, then the control processor 222 sends the moving image to the medical organization 40 via the communication unit 218, the antenna 216, and the network 36. If the camera image captured by the web camera 30 comprises a series of still images captured at given time intervals, then each time that the control processor 222 acquires one of the still images, the control processor 222 sends the still image to the medical organization 40 via the communication unit 218, the antenna 216, and the network 36.

During and after steps S10 and S11, therefore, the portable information terminal 34 continuously or intermittently sends camera images to the medical organization 40 by way of wireless communications. During and after step S12, the medical organization 40 continuously or intermittently receives camera images captured by the web camera 30 through the antenna 102 and the communication unit 104. Such continuously or intermittently captured camera images are successively stored in the memory 224 of the portable information terminal 34 and the memory 228 of the console 106.

The web camera 30 may capture a camera image (still image) at a certain time. For example, if the operator 32 operates the operating unit 60 to control the web camera 30 to capture a still image at a certain time, the control processor 222 sends the captured still image to the medical organization 40 via the communication unit 218, the antenna 216, and the network 36. In this case, the captured still image is stored in the memories 224, 228.

In the medical organization 40, in step S13 shown in FIG. 15, the web camera 116 starts capturing a camera image of the face of the doctor 38 operating the console 106, and in step S14, starts to send the captured camera image to the portable information terminal 34 via the communication unit 104, the antenna 102, and the network 36.

If the camera image captured by the web camera 116 is a moving image, then the control processor 226 sends the moving image to the medical organization 40 via the communication unit 104, the antenna 102, and the network 36. If the camera image captured by the web camera 116 is a series of still images captured at given time intervals, then each time that the control processor 226 acquires one of the still images, the control processor 226 sends the still image to the portable information terminal 34 via the communication unit 104, the antenna 102, and the network 36.

During and after step S14, therefore, the medical organization 40 continuously or intermittently sends camera images to the portable information terminal 34 by way of wireless communications. During and after step S15, the portable information terminal 34 continuously or intermittently receives the camera images captured by the web camera 116 through the antenna 216 and the communication unit 218. Such continuously or intermittently captured camera images are successively stored in the memories 224, 228.

The web camera 116 may also capture a camera image (still image) at a certain time. For example, if the doctor 38 operates the operating unit 114 and the mouse 336 to control the web camera 116 in order to capture a still image at a certain time, the control processor 226 may send the captured still image to the portable information terminal 34 via the communication unit 104, the antenna 102, and the network 36. In this case, the captured still image is stored in the memories 224, 228.

Camera images thus begin to be captured and sent and received between the medical organization 40 and the site by way of wireless communications. Then, in steps S16 and S17, the display unit 64 of the portable information terminal 34 and the display unit 112 of the console 106 start to display the camera images, etc.

FIGS. 23 through 30 show the display content of a screen 400 of the display unit 112 (the console 106) in step S17. All of the display content is processed by the control processor 226. FIGS. 31 through 39 show the display content of a screen 430 of the display unit 64 (the portable information terminal 34) in step S16. All of the display content is processed by the control processor 222.

As shown in FIGS. 23 and 24, the screen 400 of the display unit 112 displays a window 402 including a left display area, which serves as an on-site image display area 404 for displaying a camera image of the site captured by the web camera 30 (see FIGS. 1, 2, 4 through 6, and 12), and a right display area, which serves as an image capturing menu display area 406 for displaying an image capturing menu.

The image capturing menu display area 406 is displayed as a relatively large display area, whereas the on-site image display area 404 is displayed as a relatively small display area. More specifically, since the doctor 38 needs to set the content of the image capturing menu while monitoring the situation at the site by observing the camera image of the site in steps S18 through S21, to be described later, the control processor 226 (see FIG. 13) performs a display process for displaying the image capturing menu display area 406 in a size that is greater than the size of the on-site image display area 404. The on-site image display area 404 displays a camera image of the subject 18, which includes the pupils and face of the subject 18, for example.

The image capturing menu display area 406 includes an order information display area 408 for displaying order information, and a procedure display area 410 for displaying procedures. As shown in FIGS. 23 and 24, since information to be displayed in the image capturing menu display area 406 is too large to fit within the size of the image capturing menu display area 406, the image capturing menu display area 406 is relatively long in the vertical direction of the screen 400. The window 402 further includes a scroll bar on the right side thereof for vertically moving the image capturing menu display area 406 on the screen 400. If the doctor 38 operates the mouse 336 (see FIGS. 1 and 13) to place a mouse pointer (a cursor indicated by the arrow “→”) displayed on the screen 400 over the scroll bar, and then clicks on and moves the mouse 336, the control processor 226 performs a display process for vertically moving the content displayed in the image capturing menu display area 406.

The order information display area 408 displays “SIMPLE X-RAY IMAGE CAPTURE” and “RIGHT HAND IMAGE CAPTURE” as part of the order information. The procedure display area 410 displays a list of procedures for capturing images of the hand, using icons 412 through 418, depending on the content displayed in the order information display area 408. The icons 412 through 418 include pictures showing regions to be imaged of the subject 18, and simple explanations of imaging methods corresponding to the pictures.

The icon 412 indicates that the region to be imaged is a left hand and that the back of the left hand is to be irradiated with radiation 12, using a picture of the left hand and the characters “LEFT HAND WITH BACK IRRADIATED”. The picture illustrates the fingernails in order for the doctor 38 to easily distinguish between the back of the left hand and the palm of the left hand.

The icon 414 indicates that the region to be imaged is a right hand and that the back of the right hand is to be irradiated with radiation 12, using a picture of the right hand and the characters “RIGHT HAND WITH BACK IRRADIATED”. The picture illustrates the fingernails in order for the doctor 38 to easily distinguish between the back of the right hand and the palm of the right hand.

The icon 416 indicates that the region to be imaged is a left hand and that the palm of the left hand is to be irradiated with radiation 12, using a picture of the left hand and the characters “LEFT HAND WITH PALM IRRADIATED”. The picture illustrates palm lines in order for the doctor 38 to easily distinguish between the palm of the left hand and the back of the left hand.

The icon 418 indicates that the region to be imaged is a right hand and that the palm of the right hand is to be irradiated with the radiation 12, using a picture of the right hand and the characters “RIGHT HAND WITH PALM IRRADIATED”. The picture illustrates palm lines in order for the doctor 38 to easily distinguish between the palm of the right hand and the back of the right hand.

In the foregoing manner, the icons 412 through 418 include pictures of regions to be imaged and characters concerning imaging methods corresponding to the pictures. Therefore, the doctor 38 operates the mouse 336 in order to place the mouse pointer displayed in the screen 400 on one of the icons 414 through 418 while viewing the order information displayed in the order information display area 408, and then clicks on the mouse 336 to select one of the procedures according to the order information (step S18, FIG. 40).

The image capturing menu display area 406 is displayed in a large size, and the sections of the icons 412 through 418, which include the pictures, are larger than the sections thereof that include the characters. The pictures are schematically representative of actual regions to be imaged. Consequently, the doctor 38 can easily identify one of the procedures according to the order information. Furthermore, since the camera image of the site is displayed in the on-site image display area 404, the doctor 38 also can easily grasp the situation at the site (e.g., the state of the subject 18) before a radiographic image of the subject 18 is captured.

According to the present embodiment, as described above, the image capturing menu and the camera images are displayed in a window format, similar to the case of displayed screens on general personal computers. The display content of the screen 400 shown in FIGS. 23 and 24 may also be changed to the display content shown in FIGS. 25 through 30.

In FIG. 25, almost the entirety (the order information display area 408) of the window 402 is assigned to the image capturing menu display area 406, and a switching button 420 is displayed on a lower end thereof for switching to display of a camera image captured by the web camera 30. If the doctor 38 operates the mouse 336 in order to place the mouse pointer displayed in the screen 400 on the switching button 420 and then clicks on the mouse 336, as shown in FIG. 26, the control processor 226 performs a display process for displaying the on-site image display area 404 in a larger size instead of the order information display area 408. At this time, a switching button 422 for switching to display of an image capturing menu is displayed on the lower end of the window 402 in place of the switching button 420.

In FIG. 26, if the doctor 38 clicks on the mouse 336 after having operated the mouse 336 to place the mouse pointer displayed in the screen 400 over the switching button 422, the control processor 226 performs a display process for changing from the display content shown in FIG. 26 to the display content shown in FIG. 25.

In FIGS. 27 and 28, the screen 400 displays another window 424, which is of substantially the same size as the window 402. At this time, the window 402 includes (the order information display area 408 of) the image capturing menu display area 406. The window 424 includes the on-site image display area 404. If the doctor 38 operates the mouse 336 in order to place the mouse pointer displayed in the screen 400 on a desired one of the displayed windows and then clicks on the mouse 336, the control processor 226 performs a display process for bringing the clicked window to a foremost position.

In FIGS. 29 and 30, the screen 400 displays two windows 402, 424. If the doctor 38 operates the mouse 336 in order to place the mouse pointer displayed in the screen 400 on a desired one of the displayed windows and then clicks on the mouse 336, the clicked window is displayed as a full-screen window (displayed fully on the screen 400). For example, if the doctor 38 clicks on the window 402 with the mouse 336, the control processor 226 performs a display process for displaying the window 402 as a full-screen window while hiding the window 424 behind the full-screen window 402 (see FIG. 30). If the doctor 38 clicks on the window 402 again with the mouse 336, the control processor 226 changes the display content of the screen 400 back to the display content shown in FIG. 29.

As shown in FIG. 31, the screen 430 of the display unit 64 of the portable information terminal 34 displays the window 432. The window 432 includes an upper left display area that serves as an image capturing menu display area 434 for displaying an image capturing menu, a lower left display area that serves as a waiting location image display area 436 for displaying a camera image of the doctor 38 captured by the web camera 116, and a right display area that serves as an on-site image display area 438 for displaying a camera image of the site, which is captured by the web camera 30.

In FIG. 31, the on-site image display area 438 is displayed as a relatively large display area, whereas the image capturing menu display area 434 and the waiting location image display area 436 are displayed as relatively small display areas. More specifically, since the operator 32 needs to grasp the situation at the site by observing a camera image of the site, the control processor 222 performs a display process for displaying the on-site image display area 438 in a size which is greater than the size of the image capturing menu display area 434.

Since an image capturing menu has not been sent from the medical organization 40 to the portable information terminal 34, the image capturing menu display area 434 displays the characters “NOT REGISTERED AT PRESENT”, thereby indicating that an image capturing menu has not yet been established (received). Therefore, the operator 32 at the site can easily recognize that an image capturing menu has not been established, since the portable information terminal 34 has not received an image capturing menu.

The on-site image display area 438 displays the same camera image as the camera image that is displayed in the on-site image display area 404 described above (see FIGS. 23 and 24). The on-site image display area 438 is displayed as a relatively large display area, as described above. Therefore, the operator 32 can easily grasp the situation at the site before a radiographic image is captured. Since the camera image of the doctor 38 is displayed in the waiting location image display area 436, the operator 32 recognizes that the doctor 38 at the medical organization 40 is paying attention, and hence the operator 32 can feel confident while working at the site.

Since the screen 430 displays the image capturing menu and the camera images in a window format, the display content of the screen 430 shown in FIG. 31 may be changed to display content shown in FIGS. 32 through 39.

In FIG. 32, almost the entirety of the window 432 is assigned to the on-site image display area 438, with one switching button 440 being displayed at the lower end thereof for switching to display of an image capturing menu, and another switching button 442 being displayed at the lower end thereof for switching to display of the camera image captured by the web camera 116. If the operator 32 operates the operating unit 60 in order to place a mouse pointer displayed on the screen 430 over the switching button 442 and then selects the switching button 442, as shown in FIG. 33, the waiting location image display area 436 is displayed in a larger size instead of the on-site image display area 438. At this time, the switching button 444 for switching to display of a camera image of the site is displayed at the lower end of the window 432 in place of the switching button 442.

If the operator 32 operates the operating unit 60 in order to place the mouse pointer displayed on the screen 430 over the switching button 440 and then selects the switching button 440, the image capturing menu display area 434 is displayed in a larger size, instead of the waiting location image display area 436 and the on-site image display area 438. At this time, both of the switching buttons 442, 444 are displayed at the lower end of the window 432.

Similar to the case with the control processor 226, the control processor 222 can change the display content of the screen 430 of the display unit 64 to the display content shown in any one of FIGS. 32 through 34.

In FIGS. 35 through 37, the screen 430 displays other windows 446, 448, which are substantially of the same size as the window 432. At this time, the window 432 includes the on-site image display area 438, the window 446 includes the waiting location image display area 436, and the window 448 includes the image capturing menu display area 434. If the operator 32 operates the operating unit 60 in order to place a mouse pointer displayed on the screen 430 over a desired one of the displayed windows and then selects the window, the control processor 222 performs a display process for bringing the clicked window to a foremost position.

As shown in FIGS. 38 and 39, the screen 430 displays three windows 432, 446, 448. If the doctor 38 operates the operating unit 60 in order to lace the mouse pointer displayed in the screen 430 on a desired one of the displayed windows and then selects the window, the selected window is displayed as a full-screen window (displayed fully on the screen 430). For example, if the operator 32 clicks on the window 432 with the operating unit 60, the control processor 222 performs a display process for displaying the window 432 as a full-screen window while hiding the windows 446, 448 behind the full-screen window 432 (see FIG. 39). If the operator 32 selects the window 432 again with the operating unit 60, the control processor 222 changes the display content of the screen 430 back to the display content shown in FIG. 38.

In step S18 shown in FIG. 15, if the doctor 38 (see FIG. 1) selects a procedure according to the image capturing menu, the control processor 226 (see FIG. 13) changes from the display content of the screen 400 of the display unit 112 to the display content shown in FIGS. 40 and 41.

In FIGS. 40 and 41, the image capturing menu display area 406 displays an icon 450 indicative of the order information, a procedure display area 410, which displays pictures and characters of the selected icon 412 (see FIG. 24) in a larger size, and an image capturing condition display area 452, which displays image capturing conditions depending on the selected icon 412 (procedure). The procedure display area 410, which is a relatively large display area, includes the description, “BACK OF RIGHT HAND IS IRRADIATED WITH RADIATION”, which provides more detail than the description of the icon 412.

If the doctor 38 operates the mouse 336 in order to place the mouse pointer on the icon 450 and then clicks on the mouse 336, the screen 400 displays the order information display area 408, as shown in FIG. 23. The image capturing condition display area 452 displays, by way of icons 454 through 458, a list of image capturing conditions, i.e., voltages and currents of the radiation source 14, and irradiation times for the radiation 12 (see FIGS. 1, 2, 3B through 6, 8, and 12) for the selected procedure (the picture and characters of the icon 412). Since the subject 18 is identified in advance, if the thickness of the region to be imaged (right hand) and the SID are known, then the image capturing condition display area 452 also displays information concerning the thickness of the region to be imaged and the SID.

The doctor 38 operates the mouse 336 in order to place the mouse pointer displayed in the screen 400 on one of the icons 454 through 458 and then clicks on the mouse 336 while viewing the procedure displayed in the procedure display area 410 together with the thickness of the region to be imaged and the SID that are displayed in the image capturing condition display area 452, thereby selecting image capturing conditions depending on the selected procedure (step S19 shown in FIG. 15).

As a result, as shown in FIG. 42, the image capturing condition display area 452 displays a list of selected settings, and the image capturing menu display area 406 displays, in a lower portion thereof, selection buttons 460, 462 for deciding that the selected settings are OK or need to be corrected.

If the doctor 38 determines that the selected settings are OK and clicks on the selection button 460 (step S20: YES), then an image capturing menu is established, which includes the order information, the procedure, and the image capturing conditions according to the settings selected by the doctor 38, and the display content of the window 402 changes in order to the display the content shown in FIG. 43, which indicates that “ORDER INFORMATION, PROCEDURE, AND IMAGE CAPTURING CONDITIONS ARE ESTABLISHED ACCORDING TO SELECTED SETTINGS”, thereby showing that the image capturing menu has been established (step S21).

Upon elapse of a given period of time after the display content shown in FIG. 43 is displayed, the display content of the window 402 changes to the display content shown in FIG. 44. In FIG. 44, the image capturing menu display area 406 is displayed in a smaller size as a left display area of the window 402, whereas the on-site image display area 404 is displayed in a larger size as a right display area of the window 402. In steps S18 through S21 shown in FIG. 15, the control processor 226 performs a display process for displaying the image capturing menu display area 406 in a larger size, in order to allow the doctor 38 to make settings reliably for the image capturing menu. After the doctor 38 has made the image capturing menu settings, the control processor 226 performs a display process for displaying the on-site image display area 404 in a larger size in preparation for capturing of a radiographic image of the subject 18. The control processor 226 then stores the image capturing menu settings in the memory 228.

The image capturing menu display area 406 displays an icon 450 indicating the order information, an icon 464 indicating the procedure, and an icon 466 indicating the image capturing conditions. If the doctor 38 operates the mouse 336 to click on a desired one of the icons, the image capturing menu display area 406 displays information depending on the clicked icon. Therefore, the doctor 38 can easily confirm the settings of the image capturing menu.

If the settings of the image capturing menu need to be corrected in step S20, then the doctor 38 clicks on the selection button 462 (step S20: NO), whereupon the control processor 226 returns to step S18 in which the display content shown in FIGS. 23 and 24 is displayed in the window 402.

For selecting image capturing conditions in the image capturing condition display area 452, the display content shown in FIGS. 45 through 49 may be displayed instead of the display content shown in FIGS. 41 and 42.

FIG. 45 shows the display content of the image capturing condition display area 452 at a time that the doctor 38 sets a thickness of the region to be imaged and an SID while observing the subject 18 who is included in the camera image displayed in the on-site image display area 404. The image capturing condition display area 452 shown in FIG. 45 is applicable to a situation in which the thickness of the region to be imaged and the SID are to be changed.

As shown in FIG. 45, the image capturing condition display area 452 includes a text box 470 for entering the thickness of the region to be imaged, a text box 472 for entering the SID, and selection buttons 474, 476 for indicating that the entered contents are OK or need to be corrected. The doctor 38 operates the operating unit 114 to enter the thickness of the region to be imaged and the SID respectively in text boxes 470 and 472. Thereafter, if the doctor 38 clicks on the selection button 474, the display content shown in FIG. 45 changes to the display content shown in FIG. 41. If the doctor 38 clicks on the selection button 476, the content entered in the text boxes 470, 472 is erased.

In FIG. 46, the image capturing condition display area 452 includes a text box 480 for entering the thickness of the region to be imaged, spin buttons 482, 484 for incrementing and decrementing the numerical value shown in the text box 480, a text box 486 for entering the SID, spin buttons 488, 490 for incrementing and decrementing the numerical value shown in the text box 486, and the selection buttons 474 and 476.

The doctor 38 operates the operating unit 114 or uses the mouse 336 in order to click on the spin buttons 482, 484, 488, 490, thereby setting desired numerical values in the text boxes 480, 486. After having set the desired numerical values, if the doctor 38 clicks on the selection button 474, the display content shown in FIG. 46 changes to the display content shown in FIG. 41. If the doctor 38 clicks on the selection button 476, the entered content in the text boxes 480, 486 is erased, or the numerical values before the spin buttons 482, 484, 488, 490 are clicked (initial values) are displayed in the text boxes 480, 486.

Voltages and currents of the radiation source 14 and irradiation times of the radiation 12 may also be set according to the setting methods shown in FIGS. 47 through 49, rather than the setting method shown in FIG. 41, which uses the icons 454 through 458.

In FIG. 47, the image capturing condition display area 452 includes a text box 500 for entering a voltage value, a text box 502 for entering a current value, a text box 504 for entering an irradiation time, and selection buttons 506, 508 for indicating that the entered contents are OK or need to be corrected.

The doctor 38 operates the operating unit 114 in order to enter numerical values in the text boxes 500 through 504. Thereafter, if the doctor 38 clicks on the selection button 506, the display content shown in FIG. 47 changes to the display content shown in FIG. 42. If the doctor 38 clicks on the selection button 508, the entered content in the text boxes 500 through 504 is erased.

In FIG. 48, the image capturing condition display area 452 includes a text box 510 for entering a voltage value, spin buttons 512, 514 for incrementing and decrementing a numerical value shown in the text box 510, a text box 516 for entering a current value, spin buttons 518, 520 for incrementing and decrementing a numerical value shown in the text box 516, a text box 522 for entering an irradiation time, spin buttons 524, 526 for incrementing and decrementing a numerical value shown in the text box 522, and the selection buttons 506 and 508.

The doctor 38 operates the operating unit 114 or uses the mouse 336 in order to click on the spin buttons 512, 514, 518, 520, 524, 526, thereby setting desired numerical values in the text boxes 510, 516, 522. After the desired numerical values have been set, if the doctor 38 clicks on the selection button 506, the display content shown in FIG. 48 changes to the display content shown in FIG. 42. If the doctor 38 clicks on the selection button 508, the content entered in the text boxes 510, 516, 522 is erased, or the numerical values before the spin buttons 512, 514, 518, 520, 524, 526 are clicked (initial values) are displayed in the text boxes 510, 516, 522.

The image capturing condition display area 452 shown in FIG. 49 differs from the image capturing condition display area 452 shown in FIG. 48, in that an mAs value (current×irradiation time) is set instead of a current value and an irradiation time value. The image capturing condition display area 452 shown in FIG. 49 also includes the text box 510 and the spin buttons 512, 514, a text box 528 for entering an mAs value, spin buttons 530, 532 for incrementing and decrementing a numerical value shown in the text box 528, and the selection buttons 506 and 508.

The doctor 38 operates the operating unit 114 or uses the mouse 336 in order to click on the spin buttons 512, 514, 530, 532, thereby setting desired numerical values in the text boxes 510, 528. After the desired numerical values have been set, if the doctor 38 clicks on the selection button 506, the display content shown in FIG. 49 changes to the display content shown in FIG. 42. If the doctor 38 clicks on the selection button 508, the content entered in the text boxes 510, 528 is erased, or the numerical values before the spin buttons 512, 514, 530, 532 are clicked (initial values) are displayed in the text boxes 510, 528.

Upon elapse of a given period of time after the display content has been changed to the content shown in FIG. 44, in step S22 (see FIG. 16), the control processor 226 (see FIG. 13) sends the content of the image capturing menu, which was set in step S21, to the portable information terminal 34 by way of wireless communications via the communication unit 104, the antenna 102, and the network 36 (see FIGS. 1 and 12). The screen 400 of the display unit 112 then displays the characters “SETTINGS HAVE BEEN SENT TO SITE”, thereby indicating that the image capturing menu has been set in the image capturing menu display area 406, as shown in FIG. 50. The displayed characters will be erased upon elapse of a given period of time, thereby changing the screen 400 to the display content shown in FIG. 41.

In step S23, if the portable information terminal 34 receives the image capturing menu via the antenna 216 and the communication unit 218, the control processor 222 changes the display content of the screen 430 of the display unit 64 to the display content shown in FIGS. 51 and 52. In FIGS. 51 and 52, an upper left area of the window 432 is assigned to the on-site image display area 438, a lower left area is assigned to the waiting location image display area 436, and a right area is assigned to the image capturing menu display area 434, which displays the received image capturing menu.

The control processor 222 displays the image capturing menu display area 434 (the image capturing menu) in a larger size, and displays the on-site image display area 438 (the on-site camera image) and the waiting location image display area 436 (the camera image of the medical organization 40) in a smaller size. More specifically, in order for the operator 32 to confirm the content of the image capturing menu set by the console 106, the control processor 222 performs a display process for displaying the image capturing menu display area 434 in a larger size than the on-site image display area 438 and the waiting location image display area 436.

The image capturing menu display area 434 includes an order information display area 540 for displaying order information, a procedure display area 542 for displaying a procedure, and a confirmation button 546 for indicating that the operator 32 has confirmed the settings shown by the image capturing menu. In other words, the image capturing menu display area 434 displays essentially the same content (the image capturing menu) as the content of the image capturing menu display area 406.

As shown in FIGS. 51 and 52, the image capturing menu display area 434 displays the procedure display area 542 in the largest size in order to allow the operator 32 to confirm a procedure with ease, while the order information display area 540, the procedure display area 542, and the confirmation button 546 are displayed in smaller sizes. By visually recognizing a large picture, which is displayed in the procedure display area 542, and characters displayed below the picture in the procedure display area 542, the operator 32 can easily grasp that the region to be imaged of the subject 18 is the right hand, and that the back of the right hand is to be irradiated with radiation 12.

After having confirmed the image capturing menu, which is displayed in the image capturing menu display area 406 (step S24), the operator 32 operates the operating unit 60 in order to place the mouse pointer displayed in the screen 430 over the confirmation button 546, and then clicks on the confirmation button 546. The control processor 222 sends a confirmation notice indicating that the operator 32 has confirmed the image capturing menu to the medical organization 40 by way of wireless communications via the communication unit 218, the antenna 216, and the network 36 (step S25). Thereafter, as shown in FIG. 53, the control processor 222 displays the characters “CONFIRMATION NOTICE HAS BEEN SENT TO HOSPITAL” in the image capturing menu display area 434, thereby indicating to the operator 32 that the confirmation notice has been sent. The control processor 222 stores the image capturing menu in the memory 224.

Upon elapse of a given period of time after the above characters have been displayed, the control processor 222 changes the display content shown in FIG. 53 to the display content shown in FIG. 54. In FIG. 54, an upper left area of the window 432 is assigned to the image capturing menu display area 434, a lower left area is assigned to the waiting location image display area 436, and a right area is assigned to the on-site image display area 438. More specifically, since various processes are carried out on the subject 18 (see FIGS. 1, 2, 4 through 6, and 12) at the site in steps S27 through S29, and in steps S37 through S40 (see FIGS. 16 and 17) after step S25, the control processor 222 performs a display process for displaying the on-site image display area 438 in a larger size, while the image capturing menu display area 434 and the waiting location image display area 436 are displayed in a smaller size for the purpose of performing such processes efficiently.

The image capturing menu display area 434 includes the procedure display area 542, which is displayed in a relatively large size, an icon 550 indicating order information, and an icon 552 indicating image capturing conditions. If the operator 32 selects the icons 550, 552, the information indicated by the selected icons 550, 552 is displayed in the image capturing menu display area 434.

In step S26, the console 106 receives a confirmation notice via the antenna 102 and the communication unit 104 (see FIGS. 1 and 13). The control processor 226 of the console 106 changes the display content in the window 402 shown in FIG. 44 to the display content in the window 402 shown in FIG. 55, and displays the characters “SETTINGS HAVE BEEN CONFIRMED AT SITE”, thereby indicating that the confirmation notice has been received in the image capturing menu display area 406. At this time, the doctor 38 can easily grasp that the operator 32 has confirmed the settings of the image capturing menu.

In step S24, since it is only necessary for the procedure to be displayed in a large size in the image capturing menu display area 434, the display content in the window 432 may be the content shown in FIG. 56 rather than the content shown in FIGS. 51 and 52. In FIG. 56, the image capturing menu display area 434 includes an icon 550 indicating order information, an icon 552 indicating image capturing conditions, the procedure display area 542, and the confirmation button 546.

In step S27, the operator 32 locates a subject 18 corresponding to the order information of the image capturing menu at the site. Then, the operator 32 brings the subject 18 in front of the web camera 30 and captures a camera image of the subject 18 (including the subject's pupils) (step S28). In step S29, the control processor 222 sends the camera image captured in step S28 to the medical organization 40 by way of wireless communications via the communication unit 218, the antenna 216, and the network 36.

Upon the control processor 226 receiving the camera image via the antenna 102 and the communication unit 104 (step S30), the authentication processor 344 performs an authentication process for judging whether or not the subject 18 included in the camera image captured by the web camera 30 and the subject 18 represented by the order information of the image capturing menu are in agreement with each other (step S31).

In step S31, the authentication processor 344 performs a known biological authentication process using the image of the face of the subject 18 or, more preferably, by way of a known pupil authentication process using the pupils of the subject 18.

If the result of the authentication process indicates that the subject 18 included in the camera image and the subject 18 represented by the order information are in agreement with each other (step S31: YES), then the authentication processor 344 determines that an appropriate radiographic image according to the image capturing menu has been obtained by capturing an appropriate radiographic image of the subject 18 included within the camera image, and as shown in FIG. 57, displays the decision result (authentication result) in the on-site image display area 404. The doctor 38 confirms the authentication result and operates the mouse 336 to click on the confirmation button 560 (step S32), whereupon the control processor 226 sends the authentication result to the portable information terminal 34 by way of wireless communications via the communication unit 104, the antenna 102, and the network 36 (step S33).

If the result of the authentication process indicates that the subject 18 included in the camera image and the subject 18 represented by the order information are not in agreement with each other (step S31: NO), then the authentication processor 344 determines that the subject 18 included in the camera image differs from the true subject 18 represented by the order information, and as shown in FIG. 58, displays such a decision result (authentication result) in the on-site image display area 404. The doctor 38 confirms the authentication result and operates the mouse 336 to click on the confirmation button 562 (step S34), whereupon the control processor 226 sends the authentication result to the portable information terminal 34 by way of wireless communications via the communication unit 104, the antenna 102, and the network 36 (step S33).

If the control processor 222 receives the authentication result via the antenna 216 and the communication unit 218 (step S35), the control processor 222 displays an authentication result in the on-site image display area 438. If the authentication result indicates that the subject 18 included in the camera image captured by the web camera 30 (see FIGS. 1, 2, 4 through 6, and 12) and the subject 18 represented by the order information agree with each other (i.e., that they are the same person) (step S36: YES), then as shown in FIG. 59, the operator 32 confirms the displayed content and thereafter carries out a sequence during and after step S37, as shown in FIG. 17.

If the authentication result indicates that the subject 18 included in the camera image captured by the web camera 30 and the subject 18 represented by the order information are different from each other (i.e., that the subject 18 included in the camera image is not a true subject) (step S36: NO), then as shown in FIG. 60, the operator 32 carries out the process of finding the true subject 18 again in step S27.

If the operator 32 turns on the switch 50 of the cassette device 22 (see FIGS. 1, 2, 4 through 7, 9, 11, and 12) in step S37 shown in FIG. 17, the battery 166 supplies electric power to the radiation detector 20, the cassette controller 168, and the communication unit 170, thereby activating the cassette device 22 in its entirety. The cassette controller 168 sends an activation notice signal, which indicates that the cassette device 22 has been activated, to the portable information terminal 34 by way of wireless communications. If the operator 32 grips the handle 310 with one hand, the touch sensor 312 (see FIGS. 3A through 6, 8, and 12) outputs a detection signal.

Based on the received activation notice signal, the control processor 222 sends an image capturing preparation command signal for image capturing preparations, and the image capturing conditions of the image capturing menu registered in the memory 224 to the radiation source device 16 and the cassette device 22 by way of wireless communications.

The battery 134 of the radiation source device 16 continuously supplies electric power to the communication unit 136 and the radiation source controller 138. Therefore, if the radiation source controller 138 receives the image capturing preparation command signal and the image capturing conditions, the radiation source controller 138 registers the image capturing conditions, and then controls the battery 134 to supply electric power to the irradiated field lamp 56 and the web camera 330.

Upon supplying the irradiated field lamp 56 with the electric power from the battery 134, the irradiated field lamp 56 emits irradiation light 54 (see FIGS. 5 and 8). The irradiation light 54 is reflected by the mirror 144 toward the collimator 146, and is output from the radiation source device 16 and applied to the irradiated surface 44 of the cassette device 22 (step S38).

If the imaging distance is adjusted to the SID, then the irradiated field of radiation 12 that is displayed on the irradiated surface 44 by application of the irradiation light 54 is substantially in agreement with the outer frame of the guide lines 46. On the other hand, if the position of the irradiated field, i.e., the range irradiated by the irradiation light 54, is not in agreement with the position of the outer frame of the guide lines 46, or if the size of the irradiated field is not in agreement with the size of the outer frame of the guide lines 46, then the operator 32 adjusts the positional relationship between the radiation source device 16 and the cassette device 22 in order to bring the imaging distance and the SID into agreement with each other.

The image capturing preparation command signal and the image capturing conditions also are sent to the cassette device 22, so as to enable the cassette controller 168 to recognize that the radiographic image capturing apparatus 10 has been in an image capturing preparation stage, and also to register image capturing conditions in the cassette ID memory 214.

It has been described above that the radiation detector 20 is activated by turning on the switch 50. However, the battery 166 may also supply electric power (bias voltage Vb) to the radiation detector 20 in order to activate the radiation detector 20 upon receipt of the image capturing preparation command signal by the cassette controller 168. Alternatively, if the communication unit 136 of the radiation source device 16 sends a detection signal from the touch sensor 312 by way of wireless communications, then the cassette controller 168 may control the battery 166 to start supplying electric power to the radiation detector 20 upon the communication unit 170 receiving the detection signal by way of wireless communications.

The web camera 330 starts to capture a camera image of the predetermined imaging area 332, which includes the outer frame of the guide lines 46 of the cassette device 22, and sends the captured camera image to the portable information terminal 34 via the communication unit 136 and the antenna 340. The control processor 222 of the portable information terminal 34 sends the received camera image to the medical organization 40 by way of wireless communications via the communication unit 218, the antenna 216, and the network 36, and stores the camera image in the memory 224 (step S39).

If the camera image captured by the web camera 330 is a moving image, then the control processor 222 sends the moving image to the medical organization 40 via the communication unit 218, the antenna 216, and the network 36.

If the camera image is a series of still images captured at given time intervals, then each time that the control processor 222 acquires one of the still images, the control processor 222 sends the still image to the medical organization 40 via the communication unit 218, the antenna 216, and the network 36.

During and after steps S38 and S39, therefore, the portable information terminal 34 continuously or intermittently sends the camera image to the medical organization 40 by way of wireless communications. During and after step S41, the medical organization 40 continuously or intermittently receives the camera image, which was captured by the web camera 330, via the antenna 102 and the communication unit 104.

If the web camera 330 captures a camera image at a certain time, the control processor 222 sends the captured camera image to the medical organization 40 via the communication unit 218, the antenna 216, and the network 36.

In step S40, after the imaging distance has been adjusted to the SID to bring the irradiated field of the radiation 12 into agreement with the outer frame of the guide lines 46, the operator 32 places the subject 18 in front of the irradiated surface 44 of the cassette device 22, and positions the subject 18 in order to position the region to be imaged of the subject 18 inside of the outer frame of the guide lines 46.

The web camera 30 captures a camera image of the imaging area 28, which covers the region (right hand) to be imaged of the subject 18, the radiation source device 16, and the irradiated surface 44 of the cassette device 22. The web camera 330 captures a camera image of the imaging area 332, which covers the region (right hand) to be imaged of the subject 18 and the irradiated surface 44 of the cassette device 22. If the right hand of the subject 18 is injured and a radiographic image of the right hand as a region to be imaged is to be captured, then the camera images captured by the web cameras 30 and 330 provide information of how the region to be imaged is injured.

The display unit 64 of the portable information terminal 34 displays camera images captured by the web cameras 30, 330. Since the camera images captured by the web cameras 30, 330 were sent from the portable information terminal 34 to the medical organization 40 by way of wireless communications via the network 36 (steps S11, S39), the camera images captured by the web cameras 30, 330 also are displayed on the display unit 112 of the console 106.

FIGS. 61 and 63 show camera images displayed in the window 432 on the screen 430 of the display unit 64 (see FIGS. 1 and 12). FIGS. 62 and 64 show camera images displayed in the window 402 on the screen 400 of the display unit 112 (see FIGS. 1 and 13).

The on-site image display areas 404, 438 are divided into respective portable terminal image display areas 404a, 438a for displaying camera images captured by the web camera 30 (see FIGS. 1, 2, 4 through 6, and 12), and radiation source image display areas 404b, 438b for displaying camera images captured by the web camera 330 (see FIGS. 1, 2, 3B through 6, 8, and 12).

As described above, the camera images captured by the web cameras 30, 330 are sent in real time from the portable information terminal 34 via the network 36 to the medical organization 40. Therefore, the portable terminal image display areas 404a, 438a display the same camera image captured by the web camera 30, whereas the radiation source image display areas 404b, 438b display the same camera image captured by the web camera 330. Such camera images are displayed in a larger size than the size of the content displayed in the image capturing menu display areas 406, 434 and the waiting location image display area 436.

In FIGS. 61 and 62, the region (right hand) to be imaged of the subject 18 is positioned within the outer frame of the guide lines 46. In FIGS. 63 and 64, the region to be imaged is not positioned within the outer frame.

In step S42 shown in FIG. 17, the doctor 38 judges whether or not the region to be imaged of the subject 18 is properly positioned while observing the camera images displayed respectively in the portable terminal image display area 404a and the radiation source image display area 404b.

For example, if as shown in FIGS. 61 and 62, for example, the camera image indicates that the region (right hand) to be imaged of the subject 18 is included within the outer frame of the guide lines 46, the doctor 38 decides that an appropriate radiographic image of the subject 18 will be produced by capturing a radiographic image having the positional relationship between the guide lines 46 and the region to be imaged that is currently displayed (step S42: YES). Then, by using the microphone 122 to enter a voice signal or by operating the operating unit 114 and the mouse 336, the doctor 38 indicates to the operator 32 at the site that the subject 18 is properly positioned (i.e., that image capturing preparations have been completed) (step S43).

The control processor 226 sends the voice signal detected by the microphone 122 or a signal generated by the operating unit 114 and the mouse 336 to the portable information terminal 34 by way of wireless communications via the communication unit 104, the antenna 102, and the network 36 (step S44).

Based on the signal received via the antenna 216 and the communication unit 218 (step S45), the control processor 222 of the portable information terminal 34 displays the characters “POSITIONING IS OK”, thereby indicating that image capturing preparations have been completed in the waiting location image display area 436 or the on-site image display area 438, as shown in FIG. 65 or 66. Alternatively, the control processor 222 may inform the operator 32 of completion of image capturing preparations by outputting speech sounds from the speakers 78. Therefore, the operator 32 can grasp that image capturing preparations have been completed by confirming the displayed contents in the window 432, or by hearing the speech sounds from the speakers 78 (step S46: YES).

If the characters “POSITIONING IS OK” are displayed over the image of the doctor 38 in the waiting location image display area 436, then the operator 32 feels confident knowing that the doctor 38 has confirmed completion of the image capturing preparations. Furthermore, if the characters “POSITIONING IS OK” are displayed over the camera image in the on-site image display area 438, then the operator 32 can easily understand that a radiographic image can be captured of the positioned subject 18.

If the camera image indicates that the region (right hand) to be imaged of the subject 18 is not included within the outer frame of the guide lines 46, as shown in FIGS. 63 and 64, then the doctor 38 determines that an appropriate radiographic image of the subject 18 cannot be produced by capturing a radiographic image in the positional relationship between the guide lines 46 and the region to be imaged that is currently displayed (step S42: NO). Then, the doctor 38 indicates to the operator 32 at the site that the subject 18 has not been properly positioned (i.e., that the region to be imaged is to be moved into the outer frame of the guide lines 46) by using the microphone 122 to enter a voice signal, or by operating the operating unit 114 and the mouse 336 (step S47).

The control processor 226 sends the voice signal detected by the microphone 122 or a signal generated by the operating unit 114 and the mouse 336 to the portable information terminal 34 by way of wireless communications via the communication unit 104, the antenna 102, and the network 36 (step S44).

Based on the signal received via the antenna 216 and the communication unit 218 (step S45), the control processor 222 of the portable information terminal 34 displays the characters “MOVE PATIENT'S RIGHT HAND TO CENTER”, thereby indicating that the positioning is No-Good in the waiting location image display area 436 or the on-site image display area 438, as shown in FIG. 67 or 68. Alternatively, the control processor 222 may inform the operator 32 that the positioning is No-Good by outputting speech sounds from the speakers 78. Therefore, the operator 32 can grasp that the region to be imaged must be positioned again by confirming the displayed contents in the window 432, or by hearing the speech sounds from the speakers 78 (step S46: NO).

If the characters “MOVE PATIENT'S RIGHT HAND TO CENTER” are displayed over the image of the doctor 38 in the waiting location image display area 436, then the operator 32 can immediately understand the characters as a command from the doctor 38. Furthermore, if the characters “MOVE PATIENT'S HAND TO CENTER” are displayed over the camera image in the on-site image display area 438, then the operator 32 can easily understand that a radiographic image cannot be captured of the positioned subject 18.

The fact that the positioning is OK or No-Good may be indicated to the operator 32 using the display content shown in FIG. 69 or 70, rather than the display content shown in FIGS. 65 through 68. In FIGS. 69 and 70, the waiting location image display area 436 is displayed in a larger size in a right area of the window 432, whereas the image capturing menu display area 434 and the waiting location image display area 436 are displayed in a smaller size in a left area of the window 432. Further, the characters “POSITIONING IS OK” or “MOVE PATIENT'S RIGHT HAND TO CENTER” are displayed over the image of the doctor 38, which is displayed in the waiting location image display area 436. More specifically, during the preparatory stage for capturing a radiographic image, the subject 18 needs to be positioned accurately. Therefore, the waiting location image display area 436 is displayed in a larger size, and the above characters are displayed at an enlarged scale, in order to indicate and emphasize the decision of the doctor 38 to the operator 32.

In FIGS. 61, 62, 65, 66, 69, and 71, camera images displayed in the portable terminal image display areas 404a, 438a and the radiation source image display areas 404b, 438b include the right hand, which is the region to be imaged of the subject 18, and at least a portion of the right arm of the subject 18. At least in the image capturing preparatory stage, the doctor 38 is required to instruct the operator 32 while comprehending the entire region to be imaged, whereas the operator 32 is required to perform a given process while comprehending the entire region to be imaged. Since camera images including the right hand and at least a portion of the right arm are displayed, image capturing preparations can be performed effectively.

In a case where the subject 18 is positioned, at least the camera images at the site, i.e., the camera images captured by the web cameras 30, 330, may be displayed in the window 402. Therefore, as shown in FIG. 32, only a camera image of the site may be displayed, while simultaneously the image capturing menu and the camera image captured by the web camera 116 are concealed.

If the characters “POSITIONING IS OK” or “MOVE PATIENT'S RIGHT HAND TO CENTER” are fixed phrases, then as shown in FIG. 71, selection buttons 564, 566 for deciding that positioning is OK or No-Good may be displayed, rather than a speech instruction from the doctor 38. In this manner, upon the doctor 38 simply operating the mouse 336 in order to click on the selection button 564 or the selection button 566, the medical organization 40 sends an instruction depending on the clicked selection button to the portable information terminal 34. Consequently, the work burden imposed on the doctor 38 is reduced. The window 432 on the screen 430 of the display unit 64 may display any of the content shown in FIGS. 65 through 70.

While the doctor 38 judges or assesses the positioning of the region to be imaged in step S42, the window 402 may display the display content shown in FIG. 72 or 73.

In FIG. 72, the camera image captured by the web camera 30 is displayed in a larger size, and a switching button 568 for enabling display of the camera image captured by the web camera 330 is displayed at a lower end of the window 402. If the doctor 38 operates the mouse 336 to click on the switching button 568, the camera image captured by the web camera 330 is displayed in a larger size, and a switching button 570 for enabling display of the camera image captured by the web camera 30 is displayed at a lower end of the window 402, as shown in FIG. 73. Therefore, as shown in FIG. 72 or FIG. 73, since the camera image of the site is displayed at an enlarged scale, the doctor 38 can easily judge whether or not the subject 18 has been properly positioned.

As shown in FIG. 74, after step S44, an icon 572 representative of an exposure switch may be displayed in the window 402 below the image capturing menu display area 406. As described above, the console 106 includes the exposure switch 120. However, if the exposure switch 120 cannot be used under certain circumstances, the doctor 38 may use the icon 572 to give an instruction to apply radiation 12.

In step S48 shown in FIG. 18, provided that image capturing preparations have been completed (step S42: YES, steps S43, S44 in FIG. 17), the doctor 38 turns on the exposure switch 120 shown in FIGS. 1 and 13 or clicks on the icon 572 shown in FIG. 74, which represents the exposure switch. The control signal generator 346 of the control processor 226 then generates an exposure control signal for starting emission of radiation 12 from the radiation source 14, and sends the generated exposure control signal to the portable information terminal 34 via the communication unit 104, the antenna 102, and the network 36 (step S49).

If the control processor 222 (see FIG. 12) receives the exposure control signal via the antenna 216 and the communication unit 218 (step S50), the control processor 222 generates a synchronization control signal for capturing a radiographic image of the subject 18 by synchronizing start of emission of radiation 12 from the radiation source 14 with detection and conversion of radiation 12 into a radiographic image by the radiation detector 20. The generated synchronization control signal is sent to the radiation source device 16 and the cassette device 22 via the communication unit 218 and the antenna 216.

Upon the radiation source controller 138 (see FIGS. 8 and 12) receiving the synchronization control signal through the antenna 340 and the communication unit 136, the radiation source controller 138 stops supplying electric power from the battery 134 to the irradiated field lamp 56, thereby de-energizing the irradiated field lamp 56 and stopping emission of irradiation light 54, and in step S51, the radiation source controller 138 controls the radiation source 14 to apply radiation 12 at a predetermined dose to the subject 18 according to the image capturing conditions registered in the radiation source controller 138.

In the radiation source 14, the rotating mechanism 148 is controlled by the radiation source controller 138 to rotate the rotational shaft 150 and the rotary anode 152. The power supply 142 applies a negative voltage to the field-emission-type electron source 158 based on electric power supplied from the battery 134, and the power supply 140 applies a voltage between the rotary anode 152 and the cathode 156 based on electric power supplied from the battery 134. The field-emission-type electron source 158 emits electrons, which are accelerated by the voltage applied between the rotary anode 152 and the cathode 156 and bombard the target layer 154. The surface of the target layer 154, which is bombarded with electrons, emits radiation 12 from the focus point 160, the intensity of which depends on the applied electrons.

Radiation 12 passes through the mirror 144 and the irradiation area thereof is constricted by the collimator 146, after which radiation 12 is output from the radiation source device 16 and applied to the subject 18. Radiation 12 is applied to and passes through the subject 18 for a given exposure time (irradiation time) depending on the image capturing conditions, and reaches the radiation detector 20 in the cassette device 22.

In step S52, since the radiation detector 20 (see FIGS. 4 through 6, and FIGS. 9 through 12) is of an indirect conversion type, the scintillator of the radiation detector 20 emits visible light having an intensity that depends on the intensity of the radiation 12. The pixels 180 of the photoelectric conversion layer 186 convert visible light into electric signals and store the electric signals as electric charges. The electric charges stored by the pixels 180, which are representative of a radiographic image of the subject 18, are read from the pixels 180 according to address signals, which are supplied from the address signal generator 212 of the cassette controller 168 to the line scanning driver 190 and the multiplexer 192.

More specifically, in response to the address signal supplied from the address signal generator 212, the address decoder 194 of the line scanning driver 190 outputs a selection signal to select one of the switches SW1, which supplies the control signal Von to the gates of the TFTs 188 connected to the gate line 182 corresponding to the selected switch SW1. In response to the address signal supplied from the address signal generator 212, the address decoder 200 of the multiplexer 192 outputs a selection signal to successively turn on the switches SW2, so as to switch between the signal lines 184 for thereby reading, through the signal lines 184, the electric charges stored in the pixels 180 connected to the selected gate line 182.

The electric charges read from the pixels 180 connected to the selected gate line 182 are amplified by the respective amplifiers 196, sampled by the sample and hold circuits 198, and supplied to the multiplexer 192. Based on the supplied electric charges, the multiplexer 192 generates and supplies a radiographic image signal to the A/D converter 202, which converts the radiographic image signal into a digital signal. The digital signal, which represents radiographic image information, is stored in the image memory 210 of the cassette controller 168 (step S53).

Similarly, the address decoder 194 of the line scanning driver 190 successively turns on the switches SW1 in order to switch between the gate lines 182 according to address signals supplied from the address signal generator 212. The electric charges stored in the pixels 180 connected to the successively selected gate lines 182 are read through the signal lines 184, and are processed into digital signals by the multiplexer 192 and the A/D converter 202, whereupon the digital signals are stored in the image memory 210 of the cassette controller 168 (step S53).

The radiographic image information represented by the digital signals stored in the image memory 210 is transmitted together with the cassette ID information stored in the cassette ID memory 214 to the portable information terminal 34 via the communication unit 170 by way of wireless communications. The control processor 222 of the portable information terminal 34 stores the radiographic image information and the cassette ID information received via the antenna 216 and the communication unit 218 in the memory 224, and sends the radiographic image and the cassette ID information by way of wireless communications via the communication unit 218, the antenna 216, and the network 36 (step S54). The control processor 222 also displays the radiographic image stored in the memory 224 on the screen 430 of the display unit 64 (step S55).

The control processor 226 stores the radiographic image information and the cassette ID information received via the antenna 102 and the communication unit 104 in the memory 228 (step S56), and displays the radiographic image stored in the memory 228 on the screen 400 of the display unit 112 (step S57).

FIGS. 75 and 76 show the display content of the screen 430 of the display unit 64 in step S55, and FIG. 77 shows the display content of the screen 400 of the display unit 112 in step S57.

In FIG. 75, an upper left area of the window 432 on the screen 430 is assigned to the image capturing menu display area 434, a lower left area is assigned to the waiting location image display area 436, and a right area is assigned to a radiographic image display area 580, which displays a radiographic image. At the lower end of the window 432, there are displayed a switching button 574 for displaying the camera image captured by the web camera 30, and a switching button 576 for displaying the camera image captured by the web camera 330.

As shown in FIGS. 1, 61, 62, 72, and 73, since a radiographic image of the right hand of the subject 18 is captured while the right hand is placed transversely across the irradiated surface 44 of the cassette device 22, a radiographic image, which is oriented in the same direction as the camera image, normally is displayed in the radiographic image display area 580. However, in order for the operator 32 to easily recognize the radiographic image, the control processor 222 displays the captured radiographic image in a 90°-turned orientation in the radiographic image display area 580 (see FIG. 75).

In FIG. 75, since the radiographic image is displayed in a large size, the operator 32 can easily confirm whether or not the radiographic image has been acquired. If the operator 32 operates the operating unit 60 to select one of the switching buttons 574, 576, then a camera image depending on the selected switching button is displayed in a large size instead of the radiographic image.

In FIG. 76, an upper left area of the window 432 on the screen 430 is assigned to the image capturing menu display area 434, a lower left area is assigned to the radiographic image display area 580, and a right area is assigned to the portable terminal image display area 438a and the radiation source image display area 438b, which are arranged side by side. At the lower end of the window 432, a switching button 578 is provided for displaying a camera image of the doctor 38.

Even after a radiographic image has been captured, there is a possibility that the doctor 38 may give an instruction to capture a radiographic image again, as described later. The portable terminal image display area 438a and the radiation source image display area 438b, which are displayed in a large size as shown in FIG. 76, allow the operator 32 to quickly handle such an instruction from the doctor 38 in the event it is necessary to capture a radiographic image again.

In FIG. 77, an upper left area of the window 402 on the screen 400 is assigned to the image capturing menu display area 406, and a right area is assigned to a radiographic image display area 582, which displays a radiographic image. The switching button 570 for displaying the camera image captured by the web camera 30, and the switching button 568 for displaying the camera image captured by the web camera 330 are displayed at the lower end of the window 402. The image capturing menu display area 406 includes, in addition to the icons 450, 464, and 466, selection buttons 584, 586 for judging whether or not the radiographic image displayed in the radiographic image display area 582 is an appropriate image, i.e., whether the radiographic image displayed in the radiographic image display area 582 is suitable for interpretation and for making a diagnosis of the subject 18.

Even in this case, the control processor 226 displays the captured radiographic image in a 90°-turned orientation in the radiographic image display area 582, so as to allow the doctor 38 to easily confirm the radiographic image.

In FIGS. 75 and 77, the radiographic image of the right hand as the region to be imaged is displayed in a large size across the entirety of the radiographic image display areas 580, 582. This is because other body parts (e.g., the right arm) apart from the region to be imaged do not need to be displayed, since it is only necessary to accurately judge whether or not the radiographic image of the right hand is an image suitable for interpretation to make a diagnosis of the subject 18 in step S58 (see FIG. 19), to be described later.

Therefore, the radiographic image display areas 580, 582 display the radiographic image of the right hand fully therein at an enlarged scale, compared to the camera image that includes the right hand and a portion of the right arm, which is displayed in the portable terminal image display areas 404a, 438a and the radiation source image display areas 404b, 438b (see FIGS. 61, 62, 65, 66, 69, and 71). More specifically, the radiographic image of the right hand is displayed in the radiographic image display areas 580, 582 in a larger (wider) size than the optical image of the right hand, which is displayed in the portable terminal image display areas 404a, 438a and the radiation source image display areas 404b, 438b.

In step S58 shown in FIG. 19, if the doctor 38 determines that the radiographic image is an appropriate image (step S58: YES), then the doctor 38 operates the mouse 336 to click on the selection button 584, thereby indicating that the image capturing process is OK (step S59).

In step S60, the association processor 348 (see FIG. 13) of the control processor 226 associates with each other (1) the image capturing menu for capturing the present radiographic image, (2) the camera image of the face of the true subject 18 used in steps S31, S32 shown in FIG. 16, (3) the camera image of the region to be imaged of the subject 18, the positioning of which is OK, used in steps S42, S43 shown in FIG. 17, (4) the radiographic image, (5) the cassette ID information, and (6) the camera image captured by the web camera 116 of the doctor 38 in charge of capturing the present radiographic image, and stores these items in the memory 228. The associated camera images may be either still images or moving images.

Since these items of information all are stored in the memory 228, the association processor 348 reads information (an image capturing menu, various images, and cassette ID information) to be associated from the memory 228, associates the items with each other, and stores the items in the memory 228. The association processor 348 can easily perform the association process.

Since the various items of information are associated with each other in this manner, the association between the items is clearly indicated, thus enabling the doctor 38 to interpret the radiographic image and make a diagnosis of the subject 18 efficiently. More specifically, for the doctor 38 to interpret the radiographic image and make a diagnosis of the subject 18, the doctor 38 can operate the operating unit 114 of the console 106 to enter the cassette ID information or the ID information of the subject 18. The control processor 226 searches the memory 228 in order to ascertain whether or not ID information is stored in the memory 228, which is in agreement with the entered ID information. If the control processor 226 finds ID information in agreement with the entered ID information, then the control processor 226 displays on the display unit 112 various items of information associated with the ID information. The above interpretation and diagnosis process may be performed not only by way of the console 106, but also in the RIS.

Inasmuch as the camera image of the face of the subject 18 is an image used in the actual authentication process (biological authentication process, pupil authentication process), the items of information are prevented from being associated with each other based on erroneous information produced by mistaking the subject 18. Furthermore, since the camera image of the region to be imaged of the subject 18 and the radiographic image are associated with each other, the situation of the region to be imaged (e.g., how the right hand is injured) and the radiographic image can be compared with each other.

After association between the items of information has been completed, the control processor 226 changes the display content in the window 402 to the content shown in FIG. 78, thereby asking the doctor 38 to determine whether or not the subject 18 should be continuously imaged (step S61). At this time, instead of the selection buttons 584, 586, the image capturing menu display area 406 includes selection buttons 588, 590 for selecting whether the subject 18 should be continuously imaged. If the subject 18 should not be continuously imaged, the doctor 38 operates the mouse 336 to click on the selection button 590 for not continuously imaging the subject 18 (step S61: NO).

The control processor 226 then switches from the display content shown in the window 402 to the content shown in FIG. 79, thereby asking the doctor 38 to determine whether the subject 18 should be imaged under different conditions (different order information, a different procedure, or different image capturing conditions), or whether another subject 18 should be imaged (step S62). At this time, instead of the selection buttons 588, 590, the image capturing menu display area 406 includes selection buttons 592, 594 for selecting whether or not the subject 18 is to be imaged under different conditions, or whether another subject 18 should be imaged. If an image capturing process, which differs from the present image capturing process, should not be performed, then the doctor 38 operates the mouse 336 to click on the selection button 594, thereby indicating that a different image capturing process should not be performed (step S62: NO).

After completion of the above steps S58 through S62, the control processor 226 sends instruction content indicating that the present image capturing process is OK, and that capturing of the radiographic image in the present image capturing process has been completed, to the portable information terminal 34 by way of wireless communications via the communication unit 104, the antenna 102, and the network 36.

If the control processor 222 of the portable information terminal 34 receives the instruction content via the antenna 216 and the communication unit 218 (step S64), the control processor 222 successively judges whether the instruction content indicates that the present image capturing process is OK (step S65 shown in FIG. 20), whether the instruction content indicates a continuous image capturing process (step S66), and whether the instruction content indicates an image capturing process under different image capturing conditions, or an image capturing process for capturing a radiographic image of a different subject 18 (step S67).

If the instruction content indicates that the present image capturing process is OK (step S65: YES), the instruction content does not indicate a continuous image capturing process (step S66: NO), and the instruction content does not indicate an image capturing process under different image capturing conditions or an image capturing process for capturing a radiographic image of another subject 18 (step S67: NO), then the control processor 222 determines that the present image capturing process is OK, and that capturing of the radiographic image in the present image capturing process has been completed (step S68). Then, the control processor 222 switches from the display content shown by the screen 430 of the display unit 64 to the display content shown in FIG. 80.

At this time, the waiting location image display area 436 is displayed in a larger size, instead of the on-site image display area 438 (the portable terminal image display area 438a, and the radiation source image display area 438b). In addition, the characters “IMAGE CAPTURE IS OK” are displayed over the camera image of the doctor 38. The operator 32 is now capable of grasping easily that capturing of the radiographic image of the subject 18 has been completed.

In step S69, the operator 32 operates the operating unit 60 or presses the power supply switch 76 in order to shut off the portable information terminal 34, whereupon the battery 220 stops supplying electric power to various parts in the portable information terminal 34, and wireless communications between the portable information terminal 34 and the medical organization 40 are cut off.

If the operator 32 becomes spaced from the handle 310, the touch sensor 312 stops outputting the detection signal. Therefore, the radiation source controller 138 stops supplying electric power from the battery 134 to various parts in the radiation source device 16.

At this time, wireless communications between the radiation source device 16 and the portable information terminal 34 are cut off, and the web camera 330 stops capturing the camera image.

The operator 32 presses the switch 50 in order to shut off the cassette device 22, whereupon the battery 166 stops supplying electric power from the battery 166 to various parts in the cassette device 22, and wireless communications between the cassette device 22 and the portable information terminal 34 are cut off.

In step S70, the operator 32 turns the lid 66 toward the main body 62 about the hinges 70 and the shaft 68 of the portable information terminal 34, thereby bringing the teeth 72 into the recesses 74 and folding the portable information terminal 34.

Then, the operator 32 places the radiation source device 16, the cassette device 22, and the portable information terminal 34 in the attaché case 98. The operator 32 carries the attaché case 98 back to the medical organization 40 to which the operator 32 belongs (step S71).

If a radiographic image is captured of a region to be imaged of the subject 18 but the subject 18 is not placed within the outer frame of the guide lines 46, and the captured radiographic image is displayed on the screens 400, 430 of the display units 64, 112, as shown in FIGS. 81 and 82, the doctor 38 then determines that an appropriate radiographic image could not be acquired. In this case, the doctor 38 realizes that the radiographic image needs to be captured again (step S58 in FIG. 19: NO), and operates the mouse 336 to click on the selection button 586, thereby indicating that the image capturing process is No-Good (step S72).

The control processor 226 sends instruction content indicating that a radiographic image needs to be recaptured to the portable information terminal 34 by way of wireless communications via the communication unit 104, the antenna 102, and the network 36 (step S63). Upon the control processor 222 of the portable information terminal 34 receiving the instruction content via the antenna 216 and the communication unit 218 (step S64), the control processor 222 determines whether or not the instruction content indicates that a radiographic image needs to be captured again (step S65). Since, in this case, the instruction content indicates that a radiographic image needs to be captured again (the image capturing process is No-Good) (step S65: NO), the control processor 222 switches from the display content shown on the screen 430 of the display unit 64 to the display content shown in FIG. 83.

In FIG. 83, the waiting location image display area 436 displays the camera image of the doctor 38, and also displays the characters “IMAGE CAPTURING PROCESS IS NO-GOOD. RE-IMAGING IS NEEDED”, thereby indicating over the camera image of the doctor 38 that a radiographic image needs to be recaptured. Therefore, the operator 32 reliably confirms that a radiographic image needs to be captured again. The operator 32 repositions the subject 18 and carries out preparations for capturing a radiographic image again, as indicated back in step S40 shown in FIG. 17.

As shown in FIGS. 84 and 85, the screens 400, 430 may display the on-site image display areas 404, 438 (the portable terminal image display areas 404a, 438a and the radiation source image display areas 404b, 438b) in a larger size, while displaying the improperly captured radiographic image in a smaller size in the radiographic image display areas 580, 582.

It has been described above that, in the event that the region to be imaged of the subject 18 was not positioned within the outer frame of the guide lines 46 and an erroneous radiographic image was captured, the doctor 38 instructs the operator 32 to capture a radiographic image again. However, the present embodiment is not limited to this description. Instead, the present embodiment also is applicable in cases where, although the region to be imaged is positioned within the outer frame of the guide lines 46 at the time the subject 18 is positioned, the region to be imaged moves outside of the outer frame of the guide lines 46 due to body movement of the subject 18 during capturing of a radiographic image of the subject 18. In such a case, since the radiographic image is displayed in the window 402 in a large size, as shown in FIG. 82, the doctor 38 can easily comprehend that capturing of the radiographic image is a failure, and can quickly instruct the operator 32 to recapture the radiographic image.

In step S61 shown in FIG. 19 and FIG. 78, if the doctor 38 operates the mouse 336 to click on the selection button 588, thereby indicating that the subject 18 is to be continuously imaged (step S61: YES), the medical organization 40 sends instruction content indicative of a continuous image capturing process to the portable information terminal 34 (steps S63, S64). Based on the instruction content indicative of a continuous image capturing process (step S66: YES), the control processor 222 undertakes preparations for a subsequent image capturing process, as described back in step S40 shown in FIG. 17 or step S50 shown in FIG. 18. The medical organization 40 also undertakes preparations for a subsequent image capturing process, as described back in step S42 or step S48.

In step S62 shown in FIG. 19 and FIG. 79, if the doctor 38 operates the mouse 336 to click on the selection button 592, thereby indicating that the subject 18 is to be imaged under different conditions or that a different subject 18 should be imaged (step S62: YES), the medical organization 40 sends instruction content indicating that a new image capturing process is required to the portable information terminal 34 (steps S63, S64). Based on the instruction content indicative of the new image capturing process (step S67 in FIG. 20: YES), the control processor 222 undertakes preparations for a subsequent image capturing process as described back in step S23 shown in FIG. 16. The medical organization 40 also undertakes preparations for a subsequent image capturing process, as described back in step S8 shown in FIG. 14 or step S18 shown in FIG. 15.

The first embodiment is not limited to the above description, but rather, the screens shown in FIGS. 86 through 92 may be displayed.

FIGS. 86 and 87 illustrate a case in which only the radiographic image is displayed in a larger size in the windows 402, 432, similar to the case of FIG. 32. Since the radiographic image is displayed in a larger size, the doctor 38 can easily judge whether or not the radiographic image is appropriate, and the operator 32 can easily grasp what type of radiographic image has been obtained.

The windows 402, 432 display at lower ends thereof a plurality of switching buttons 422, 440, 442, 568, 570, 574, 576. If any one of the switching buttons is selected (clicked on), information (an image) depending on the selected switching button is displayed in a larger size instead of the radiographic image. Therefore, the doctor 38 or the operator 32 can easily confirm the displayed information in a large size, whereas unwanted information is not displayed.

FIGS. 88 and 89 show a right area of the windows 402, 432, in which four images are displayed, i.e., a radiographic image, a camera image of the face of the subject 18 that is captured by the web camera 30 prior to capturing of the radiographic image (portable terminal image display areas 404c, 438c), a camera image that is captured by the web camera 30 prior to capturing of the radiographic image (an image of the radiation source device 16, the region to be imaged, and the cassette device 22 displayed in the portable terminal image display areas 404a, 438a), and a camera image that is captured by the web camera 30 before the radiographic image is captured (an image of the region to be imaged, and the cassette device 22 that is displayed in the radiation source image display areas 404b, 438b).

As described above, the association processor 348 associates (1) the image capturing menu, (2) the camera image of the face of the subject 18, (3) the camera image of the region to be imaged, (4) the radiographic image, (5) the cassette ID information, and (8) the camera image of the doctor 38 with each other, and stores these items in the memory 228. In FIGS. 88 and 89, such items of information (images), apart from the cassette ID information, are display altogether.

If the images shown in FIG. 88 are displayed in step S59 before the items of information are associated (step S60 shown in FIG. 19), then going forward, the doctor 38 can grasp the items of information to be associated, and can quickly identify the subject 18 while grasping the situation of the region to be imaged. If the images shown in FIG. 89 are displayed in step S68 shown in FIG. 20, the doctor 38 can easily understand the kinds of information that have been associated with each other. If such items of information are displayed altogether while the doctor 38 interprets the radiographic image and makes a diagnosis of the subject 18, then the doctor 38 can interpret the radiographic image and make a diagnosis of the subject 18 efficiently.

FIGS. 90 and 91 illustrate close-up images of the pupils of the subject 18, which are captured by the web camera 30 in step S28 shown in FIG. 19, and are displayed at an enlarged scale in the on-site image display areas 404, 438, which are displayed in a larger size in the windows 402, 432. The close-up images of the pupils of the subject 18 allow the authentication processor 344 to perform the pupil authentication process more accurately in step S31. As a result, items of information are reliably prevented from being associated with each other based on erroneous information produced by mistaking the subject 18.

FIG. 92 shows not only a procedure according to the order information, but also a list of all of the procedures that are registered in the memory 228, which are displayed as icons 630 through 638 in the procedure display area 410. The icons 630 through 638 include pictures showing regions to be imaged of the subject 18, and characters indicating the regions that are to be imaged. The icon 630 indicates that the region to be imaged is a hand. The icon 632 indicates that the region to be imaged is a leg. The icon 634 indicates that the region to be imaged is a lung. The icon 636 indicates that the region to be imaged is a group of ribs. The icon 638 indicates that the region to be imaged is a stomach. If the doctor 38 operates the mouse 336 to click on the icon 630, for example, the display content changes to the display content shown in FIG. 24, which indicates a detailed list of procedures concerning capturing of radiographic images of the hand.

The first embodiment has been described above.

Next, a second embodiment will be described below.

Following step S16 shown in FIG. 15, in step S81 shown in FIG. 21, the operator 32 (see FIGS. 1, 2, and 3B through 7) finds a subject 18 whose radiographic image needs to be captured due to an injury suffered at a disaster site, for example. Then, the operator 32 regards the injured region of the subject 18 as a region to be imaged, and directs the web camera 30 toward the injured region (the region to be imaged), after which the operator 32 captures a camera image of the subject 18 including the injured region (step S82). In step S83, the control processor 222 (see FIG. 12) sends the camera image captured in step S82 to the medical organization 40 by way of wireless communications via the communication unit 218, the antenna 216, and the network 36.

Upon receipt of the camera image by the control processor 226 (see FIG. 13) via the antenna 102 and the communication unit 104 (step S84), the control processor 226 switches from displaying the content shown by the screen 400 of the display unit 112 to the content shown in FIG. 93.

In FIG. 93, a left area of the window 402 is assigned to the on-site image display area 404, and a right area is assigned to the image capturing menu display area 406. The image capturing menu display area 406 displays an order information editing area 600 for enabling changes (setting, editing) to be made to the order information.

The order information editing area 600 includes a text box 602 for entering the ID of the subject 18 included within the camera image, a text box 604 for entering the name of the subject 18, a text box 606 for entering the gender of the subject 18, a text box 608 for entering the age of the subject 18, a text box 610 for entering an imaging method for the subject 18, a text box 612 for entering a region to be imaged of the subject 18, and selection buttons 614, 616 for indicating whether the entered content is OK or No-Good.

In step S85, the doctor 38 operates the operating unit 114 and the mouse 336 in order to enter necessary content into the text boxes 602 through 612 while observing the camera image of the subject 18 displayed in the on-site image display area 404. If the entered content is acceptable, then the doctor 38 operates the mouse 336 to click on the selection button 614, whereupon the order information is established (step S86) and a list of procedures depending on the order information is displayed in the image capturing menu display area 406 (step S18 shown in FIG. 15, FIGS. 23 and 24).

If the doctor 38 wishes to correct the entered content, the doctor 38 clicks on the selection button 616 in order to delete the content entered in the text boxes 602 through 612. Since the portable information terminal 34 has the microphone 80 and the speakers 78, whereas the console 106 has the microphone 122 and the speaker 118, the doctor 38 may ask the operator 32 for additional information (e.g., the name of the subject 18) concerning the content entered in the text boxes 602 through 612, and the operator 32 may answer the doctor 38 appropriately.

At a disaster site, a number of injured persons may possibly be present, and the operator 32 may need to quickly deal with such injured persons. As a result, the doctor 38 may not possibly obtain adequate answers from the operator 32, and it may be difficult to fill in all of the text boxes 602 through 612. In such a case, the doctor 38 may leave such unfilled text boxes as they are, and may enter information only in text boxes that can be filled.

According to the second embodiment, the doctor 38 needs to change the image capturing menu while observing the camera image of the site. Therefore, the on-site image display area 404 and the image capturing menu display area 406 in the window 402 preferably are of the same size.

The second embodiment has been described above.

Next, a third embodiment will be described below.

In the third embodiment, order information is established (step S86 in FIG. 21), and an image capturing menu is established based on such order information (steps S18 through S21 in FIG. 15). The established image capturing menu is sent from the medical organization 40 to the portable information terminal 34 (steps S22, S23 in FIG. 16). In step S91 shown in FIG. 22, after the operator 32 has confirmed the image capturing menu (step S24), the operator 32 judges whether the content of the confirmed image capturing menu is appropriate content corresponding to the injured region (region to be imaged) of the subject 18.

More specifically, as shown in FIG. 94, selection buttons 620, 622 for determining whether the established content is OK are displayed in a lower portion of the image capturing menu display area 434. The operator 32 judges whether or not the injured subject 18 and the content of the image capturing menu match each other. If the content of the image capturing menu is OK, then the operator 32 operates the operating unit 60 to click on the selection button 620 (step S91: YES).

The control processor 222 (see FIG. 12) performs step S25 in order to send a confirmation notice indicating that the content of the established image capturing menu is OK to the medical organization 40 by way of wireless communications via the communication unit 218, the antenna 216, and the network 36, and changes the display content in the window 432 from the content shown in FIG. 94 to the content shown in FIG. 95, whereupon the characters “OK NOTICE HAS BEEN SENT TO HOSPITAL” are displayed in the image capturing menu display area 434.

Upon the control processor 226 (see FIG. 1) of the console 106 receiving the confirmation notice via the antenna 102 and the communication unit 104, the control processor 226 changes the display content of the screen 400 of the display unit 112 to the display content shown in FIG. 96. At this time, the doctor 38 can grasp that the operator 32 has confirmed the content of the image capturing menu, and that the content of the established image capturing menu is OK.

If the injured subject 18 and the content of the image capturing menu do not match each other (step S91: NO), the operator 32 operates the operating unit 60 to click on the selection button 622 (step S92). The control processor 222 sends a confirmation notice (No-Good notice), which indicates that the content of the established image capturing menu does not match the subject 18, to the medical organization 40 by way of wireless communications via the communication unit 218, the antenna 216, and the network 36, and changes the display content in the window 432 from the content shown in FIG. 94 to the content shown in FIG. 97, thereby displaying the characters “NO-GOOD NOTICE HAS BEEN SENT TO HOSPITAL” in the image capturing menu display area 434 (step S93).

Upon the control processor 226 of the console 106 receiving the No-Good notice via the antenna 102 and the communication unit 104 (step S94), the control processor 226 changes the display content of the screen 400 of the display unit 112 to the display content shown in FIG. 98. In this manner, the doctor 38 can grasp that the operator 32 has confirmed the content of the image capturing menu, and that the image capturing menu needs to be established again. In addition to displaying the No-Good notice on the display unit 112, the console 106 may also output a sound from the speaker 118 in order to provide a warning that the image capturing menu needs to be established again (step S95).

It has been described in relation to the third embodiment whether or not the full content of the image capturing menu is appropriate at the site. However, only one individual item, such as the order information, the procedure, or the image capturing conditions, may be judged at the site, and such a judgment result may be sent to the medical organization. For example, if it is to be judged at the site whether the procedure is appropriate, the display unit 112 of the console 106 may display the characters “PROCEDURE IS OK” or “PROCEDURE IS NO-GOOD” on the screen 400 of the display unit 112.

### [Advantages of the Present Exemplary Embodiments]

The radiographic image capturing system 11, the radiographic image capturing method, and the image displaying method according to the present exemplary embodiments described above offer the following advantages.

With the radiographic image capturing system 11, the radiographic image capturing method, and the image displaying method according to the present exemplary embodiments, prior to capturing a radiographic image, the communication unit 218 of the portable information terminal 34 sends camera images of the site (optical images captured by the web cameras 30, 330) to the communication unit 104 of the medical organization 40, and the communication unit 104 sends the image capturing menu and the camera image of the medical organization 40 (the optical image captured by the web camera 116). Therefore, the display unit 112 of the console 106 can display at least one of the image capturing menu and camera images of the site, whereas the display unit 64 of the portable information terminal 34 can display at least one of the image capturing menu, camera images of the site, and the camera image of the medical organization 40.

Consequently, the doctor 38, who waits in a waiting location such as the medical organization 40 or the like, can give the operator 32 at the site an appropriate instruction (e.g., an instruction to position the subject 18) while observing camera images of the site that are displayed on the display unit 112. If the image capturing menu is displayed on the display unit 112, the doctor 38 can confirm (monitor) whether the operator 32 is working according to the content of the image capturing menu while simultaneously observing camera images of the site.

By viewing the image capturing menu displayed on the display unit 64, the operator 32 can grasp what type of radiographic image is to be captured of the subject 18, and can perform an appropriate operation (e.g., positioning of the subject 18) depending on the image capturing menu. If camera images of the site are displayed on the display unit 64, then the operator 32 can easily confirm the situation at the site. If the camera image of the medical organization 40 is displayed, the operator 32 can recognize that the medical organization 40 has grasped the situation of the site, and can work at the site with confidence.

After a radiographic image is captured, the communication unit 218 sends camera images of the site and the radiographic image to the communication unit 104. At this time, the display unit 112 of the console 106 can display at least one of the image capturing menu, camera images of the site, and the radiographic image. The display unit 64 of the portable information terminal 34 can display at least one of the image capturing menu, camera images of the site, the camera image of the medical organization 40, and the radiographic image.

In this manner, the doctor 38 can judge whether an appropriate radiographic image (an image suitable for interpretation and to make a diagnosis of the subject 18) according to the image capturing menu while observing the radiographic image displayed on the display unit 112. The operator 32 can judge whether or not the radiographic image has been captured suitably by observing the radiographic image displayed on the display unit 64.

According to the present exemplary embodiments, therefore, assuming that the doctor 38 waits in a waiting location (the medical organization 40) where the doctor 38 is unable to see the subject 18 directly, the doctor 38 can instruct the operator 32 at the site how to capture a radiographic image of the subject 18 in real time while observing the display content on the display unit 112. Consequently, a radiographic image of the subject 18 can be captured without requiring the doctor 38 to travel directly to the site.

Inasmuch as signals representative of the image capturing menu, camera images of the site, the camera image of the medical organization 40, and the radiographic image are sent and received between the communication units 104, 218, the display unit 112 is capable of displaying information on the screen 400 that the doctor 38 wants to see at present in as large a size as possible, depending on how the radiographic image is captured (before and after the radiographic image is captured). Further, the display unit 64 can display on the screen 430 information that the operator 32 wants to see at present in as large a size as possible, depending on how the radiographic image is captured. Thus, the doctor 38 can instruct the operator 32 appropriately, and the operator 32 can work efficiently at the site according to the content (an instruction from the doctor 38, etc.) displayed on the display unit 64.

Since information that the doctor 38 wants to see is displayed on the screen 400 in a large size, and the information that the operator 32 wants to see is displayed on the screen 430 in a large size depending on how the radiographic image is captured, the screens 400, 430 may display the same display content or different display content thereon.

For example, in a case where the subject 18 is positioned, the screens 400, 430 preferably display the same display content, since it is necessary to adjust the position of the region to be imaged of the subject 18 while judging whether or not the region to be imaged of the subject 18 is positioned within the outer frame of the guide lines 46. At this time, if the camera image including both the region (right hand) to be imaged of the subject 18 and a region (right arm) near the region to be imaged is displayed, the doctor 38 can appropriately instruct to the operator 32 while grasping the overall region to be imaged, and in addition, the operator 32 can perform a given operation while grasping the overall region to be imaged. As a result, image capturing preparations can be performed efficiently.

In a case where an image capturing menu is established, or after capturing of a radiographic image, the screens 400, 430 may display different content respectively.

According to the present exemplary embodiment, at the site, the web camera 330 is integral with the radiation source device 16, whereas the web camera 30 is integral with the portable information terminal 34. Accordingly, the number of parts used is reduced, thereby allowing the radiographic image capturing apparatus 10 to be assembled and accommodated easily at the site.

According to the present exemplary embodiment, furthermore, the web camera 330 captures an image of at least one of the cassette device 22 and the subject 18, whereas the web camera 30 captures an image of at least one of the cassette device 22, the radiation source device 16, and the subject 18. Further, the web camera 116 captures an image of the doctor 38.

Since the camera image captured by the web camera 330 and the camera image captured by the web camera 30 are displayed on the display unit 64, the operator 32 can grasp the situation of the subject 18, the cassette device 22, and the radiation source device 16 simply by confirming the display content of the display unit 64 without directly viewing the subject 18, the cassette device 22, and the radiation source device 16. Since the camera image captured by the web camera 116 is displayed on the display unit 64, the operator 32 can recognize that the doctor 38 is observing the site and hence can work at the site with confidence. Furthermore, since the camera image captured by the web camera 330 and the camera image captured by the web camera 30 are displayed on the display unit 112, the doctor 38 can adequately grasp the situation at the site, including the subject 18, the cassette device 22, and the radiation source device 16, and can provide appropriate instructions to the operator 32 without travelling directly to the site.

Until a radiographic image is captured, the display units 64, 112 display camera images captured by the web cameras 30, 330 or the image capturing menu at a size larger than other images. Accordingly, the subject 18 can be positioned and an image capturing menu can be established efficiently before capturing a radiographic image.

As described above, depending on how the radiographic image is captured, the display unit 112 displays information on the screen 400 that the doctor 38 wants to see in as large a size as possible, and the display unit 64 can display information on the screen 430 that the operator 32 wants to see in as large a size as possible. More specifically, the information is displayed in the following manner.

In a case where a procedure and image capturing conditions are established based on the order information on the console 106, the display unit 112 displays an image capturing menu in a larger size than other images. Therefore, the image capturing conditions can be established more easily. In the image capturing menu received via the communication unit 218, the display unit 64 displays the procedure in a larger size than the order information and the image capturing conditions, thereby indicating for sure to the operator 32 the procedure for capturing a radiographic image in a present cycle.

After a radiographic image has been captured, preferably, at least the display unit 112 displays the radiographic image in a larger size than other images. It is thus possible to easily judge whether or not an appropriate radiographic image has been obtained according to the image capturing menu. If the radiographic image of the region (right hand) to be imaged is displayed in a larger (wider) size than the camera image of the region to be imaged and the region (portion of the right arm) in the neighborhood thereof, then it is possible to accurately judge whether the radiographic image is suitable to enable interpretation of the image and to make a diagnosis of the subject 18.

If a radiographic image needs to be captured again, the display units 64, 112 may display the camera images captured by the web cameras 30, 330 or the image capturing menu in a larger size than other images. Accordingly, the time required to undertake preparations for recapturing the image can be shortened.

If a radiographic image does not need to be captured again, or if capturing of only one radiographic image is sufficient, then the display units 64, 112 need not necessarily display an image capturing menu, but may display other images in a larger size after the radiographic image has been captured.

According to the present exemplary embodiments, since the authentication processor 344 judges whether or not the subject 18 included within the camera images captured by the web cameras 30, 330 and the subject 18 indicated by the order information are in agreement with each other, items of information are prevented from being wrongly associated with each other by mistaking the subject 18, and radiographic images are prevented from being captured in error.

More specifically, the web cameras 30, 330 capture camera images of the face of the subject 18, and the authentication processor 344 determines whether or not the subject 18 included within the camera images and the subject 18 indicated by the order information are in agreement with each other based on camera images of the face, which are captured by the web cameras 30, 330. Since the authentication process is performed using facial images, it is possible to reliably judge whether or not the subject 18 included within the camera images is a true subject.

More preferably, the camera images of the face include the pupils of the subject 18. The authentication processor 344 determines whether or not the subject 18 having the pupils included within the camera images and the subject 18 indicated by the order information are in agreement with each other based on camera images that include the pupils, which are captured by the web cameras 30, 330. According to the pupil authentication process, it is possible to perform an authentication process for the subject 18 who is included within the camera images and the subject 18 indicated by the order information, thereby reliably preventing items of information from being wrongly associated with each other by mistaking the subject 18, and also preventing radiographic images from being captured by mistake.

The display units 64, 112 may display at least one of the camera images captured by the web cameras 30, 330 and the image capturing menu. This makes it easy to judge whether or not (the region to be imaged of) the subject 18 included within the camera images captured by the web cameras 30, 330 and the content of the image capturing menu correspond to each other.

Furthermore, after the radiographic image has been captured, the display units 64, 112 may display at least both a radiographic image and the image capturing menu. Thus, it is possible to efficiently judge whether or not an appropriate radiographic image has been obtained according to the image capturing menu.

With the radiographic image capturing system 11, the radiographic image capturing method, and the image displaying method according to the present exemplary embodiments, as illustrated in the second embodiment, prior to capturing a radiographic image, the communication unit 218 at the site sends a camera image of the site to the communication unit 104 of the medical organization 40 (waiting location), and the console 106 establishes (changes) the content of the image capturing menu depending on the situation of the subject 18 who is currently included within the camera image, whereupon the established image capturing menu is sent from the communication unit 104 to the communication unit 218. As a result, the portable information terminal 34 controls the radiation source device 16 and the cassette device 22 based on the established image capturing menu in order to capture a radiographic image of the subject 18.

According to the present exemplary embodiments, therefore, before a radiographic image is captured, an image capturing menu is established (changed) depending on the subject 18, and a radiographic image of the subject 18 is captured based on the established image capturing menu. Therefore, an appropriate radiographic image can be acquired.

Before a radiographic image is captured, the portable information terminal 34 determines whether or not the subject 18 included within the camera images captured by the web cameras 30, 330 and the subject 18 indicated by the image capturing menu are in agreement with each other. The control processor 222 then sends the judgment result via the communication unit 218 to the communication unit 104. At this time, the doctor 38 is capable of grasping whether the established image capturing menu is an appropriate image capturing menu that depends on the subject 18 at the site.

If the portable information terminal 34 determines that the subject 18 included within the camera images and the subject 18 indicated by the image capturing menu are in agreement with each other, the display unit 112 displays the judgment result on the screen 400, whereby the doctor 38 can easily grasp the judgment result.

After a judgment result indicating that the subjects 18 are in agreement with each other has been indicated, the doctor 38 presses the exposure switch 120 (or clicks on the icon 572), whereupon the control signal generator 346 sends an exposure control signal from the communication unit 104 to the communication unit 218. Based on a synchronization control signal, which depends on the received judgment result and the image capturing menu, the portable information terminal 34 controls the radiation source device 16 and the cassette device 22 in order to capture a radiographic image. Therefore, the radiographic image can be captured reliably.

If the judgment result indicates that the subject 18 included within the camera images and the subject 18 indicated by the image capturing menu are not in agreement with each other, then as shown in the third embodiment, the control processor 226 may display a judgment result on the screen 400 of the display unit 112, and output a sound warning from the speaker 118. Thus, it is possible to indicate to the doctor 38 that the image capturing menu does not correspond to the subject 18 at the site, thereby allowing the doctor 38 to immediately perform a process to change the image capturing menu depending on the subject 18, for example.

In this case, the console 106 changes at least one of the order information, the procedure, and the image capturing conditions in the image capturing menu depending on the situation of the subject 18 who is included within the camera images. In this manner, an appropriate image capturing menu can be established depending on the subject 18.

If the subject 18 included in the camera images is different from the subject 18 indicated by the order information, or if the subject 18 included in the camera images is in agreement with the subject 18 indicated by the order information but a different region of the subject 18 needs to be imaged, then the content of the image capturing menu may be modified such that the sequence of the order information, the procedure, and the image capturing conditions thereof are changed. If the subject 18 is different or the region to be imaged is different, then since the order information per se requires changes to be made thereto, the content of the image capturing menu is modified such that the sequence of the order information, the procedure, and the image capturing conditions thereof are changed, thereby establishing a more accurate image capturing menu.

If the subject 18 who is included within the camera images is in agreement with the subject 18 indicated by the order information, but a different procedure is involved, then the content of the image capturing menu may be changed to modify the sequence of the procedure and the image capturing conditions. In this case, a more accurate image capturing menu can be established.

The communication unit 218 may send at least a camera image of the site and a radiographic image to the communication unit 104. The display units 64, 112 may display at least one of the camera image, the radiographic image, and the image capturing menu. The doctor 38 can establish an accurate image capturing menu depending on the subject 18 while confirming the display content on the display unit 112, whereas the operator 32 can confirm the display content on the display unit 64 and can easily determine whether or not the image capturing menu depends on the subject 18.

If the image capturing menu is changed, the camera image of the site and the image capturing menu are displayed together on the display unit 112, such that the camera image of the site is displayed in a larger size than other images. Thus, the burden imposed on the doctor 38 in a case where the doctor 38 establishes the image capturing menu is reduced.

With the radiographic image capturing system 11, the radiographic image capturing method, and the image displaying method according to the present exemplary embodiments, camera images of the site (optical images captured by the web cameras 30, 330) and the radiographic image are sent from the communication unit 218 to the communication unit 104, whereas at least the image capturing menu is sent from the communication unit 104 to the communication unit 218. The console 106 or the portable information terminal 34 associate at least the image capturing menu, the radiographic image, and camera images of the site with each other, and stores these items in the memories 224, 228.

As described above, after a radiographic image has been captured, at least the image capturing menu, the radiographic image, and camera images of the site are associated with each other and stored in the memories 224, 228. Therefore, information of the subject 18 at the site and the radiographic image of the subject 18 are accurately associated with each other, thus enabling the doctor 38 to adequately and efficiently perform a process of interpreting the radiographic image and to make a diagnosis of the subject 18.

The web camera 330 captures a camera image of the cassette device 22 and the region to be imaged of the subject 18 who has been positioned with respect to the cassette device 22, whereas the web camera 330 captures camera images of the cassette device 22, the region to be imaged of the subject 18 who has been positioned with respect to the cassette device 22, and the radiation source device 16. Since the radiographic image of the subject 18 and camera images (e.g., the optical image of the region to be imaged) related to the radiographic image are associated with each other, the doctor 38 finds it easy to interpret the radiographic image and to make a diagnosis of the subject 18.

As described above, the authentication processor 344 performs a biological authentication process (pupil authentication process) using an image of the face (including the pupils) of the subject 18 in the camera image captured by the web camera 30. Therefore, incorrect items of information are prevented from being associated with each other, and as a result, reliability of the associated items of information can be increased.

The authentication processor 344 judges whether or not the subject 18 included in the camera images and the subject 18 indicated by the order information are in agreement with each other, and sends the authentication result from the communication unit 104 to the communication unit 218. The display unit 64 of the portable information terminal 34 displays the authentication result received via the communication unit 104 on the screen 430. If the subject 18 included in the associated camera images and the subject 18 indicated by the order information are not in agreement with each other, then the operator 32 can search at the site and locate the true subject 18 indicated by the order information.

In addition to the items of information (images) described above, either the console 106 or the portable information terminal 34 may associate the camera image captured by the web camera 116, and store the camera image in the memories 224, 228, thereby making it possible to associate information concerning the doctor 38 who has instructed the operator 32 how to capture the radiographic image.

The display units 64, 112 may display the image capturing menu, the radiographic image, camera images of the site, and the camera image captured by the web camera 116 in a juxtaposed relationship. Since the associated images and information are displayed together, the doctor 38 and the operator 32 can easily grasp the kinds of information that have been associated with each other.

The associated items of information preferably are stored in the memory 228 of the console 106, the memory 224 of the portable information terminal 34, the memory card 92, or the USB memory 334. More preferably, the associated items of information are stored after being encrypted.

The memory card 92 can be removably inserted in the card slot 94 of the portable information terminal 34, and the USB memory 334 can be removably connected to the USB terminals 84, 88, 90. Therefore, if the associated items of information, which have been encrypted, are stored in at least one of the memory card 92 and the USB memory 334 connected to the portable information terminal 34, the control processor 222 may display on the display unit 64 a notice for permitting removal of at least one of the memory card 92 and the USB memory 334 from the portable information terminal 34, or may output a sound representative of such a notice from the speakers 78, after the associated items of information have been stored in at least one of the memory card 92 and the USB memory 334.

Alternatively, if the portable information terminal 34 includes a lock mechanism, not shown, for preventing at least one of the memory card 92 and the USB memory 334 from being removed from the portable information terminal 34, then the control processor 222 may control the lock mechanism to cancel the removal preventing function thereof, so as to allow at least one of the memory card 92 and the USB memory 334 to be removed from the portable information terminal 34 after the associated items of information have been stored in at least one of the memory card 92 and the USB memory 334. In this case, the control processor 222 may display a memory removal permission notice on the display unit 64, or may output a sound representative of such a notice from the speakers 78.

The present exemplary embodiments offer the following advantages in addition to the advantages noted above.

At a disaster site, a home care treatment site, or the like, the web camera 30, which is integral with the portable information terminal 34 (incorporated in the portable information terminal 34), and the web camera 30, which is integral with the radiation source device 16 (incorporated in the radiation source device 16), capture camera images of at least (the guide lines 46 corresponding to the radiation detector 20 housed in) the cassette device 22, whereupon the communication unit 218 sends the camera images captured by the web cameras 30, 330 via the network 36 to the communication unit 104 of the medical organization 40.

Based on camera images received by the communication unit 104, the doctor (or radiological technician) 38 waiting in the medical organization 40 (in a remote location) and who is unable to see the subject 18 directly can instruct the operator 32 of the radiographic image capturing apparatus 10 at the site, such as the disaster site, the home care treatment site, or the like, in real time to capture a radiographic image of the subject 18. Therefore, even though the doctor 38 is not present at the site directly, i.e., even though the doctor 38 does not go with the operator 32, who is not qualified as a medical radiological technician, i.e., who doesn't have authority to apply radiation 12 to the subject 18, a radiographic image of the subject 18 can be captured.

The outer frame of the guide lines 46 corresponds to the irradiated field of radiation 12 at the time the imaging distance is set to the SID and the web camera 30 captures a camera image of the outer frame of the guide lines 46. Therefore, if the doctor 38 sees the camera image captured by the web camera 30 and if the doctor 38 finds that the region to be imaged of the subject 18 is positioned (included) within the outer frame of the guide lines 46, the doctor 38 is capable of determining that an appropriate radiographic image can be obtained by applying radiation 12 to the subject 18. If the region to be imaged of the subject 18 is positioned outside of the outer frame of the guide lines 46, or if only a portion of the region to be imaged is included within the outer frame of the guide lines 46, then the doctor 38 is capable of determining that a desired radiographic image cannot be obtained by applying radiation 12 to the subject 18.

The web camera 30 captures a camera image of the guide lines 46, and the doctor 38 sees (monitors) the camera image captured by the web camera 30 and judges whether or not the region to be imaged of the subject 18 is positioned within the outer frame of the guide lines 46, thereby judging whether an appropriate radiographic image can be obtained. As a consequence, even if the doctor 38 is unable to directly see the subject 18 and the operator 32, the doctor 38 can still instruct the operator 32 at the site how to carry out appropriate imaging preparations.

As described above, the web camera 30 is integral with the portable information terminal 34 as a result of being incorporated in the upper surface of the lid 66 of the portable information terminal 34. Since the web camera 30 captures a camera image of the radiation source device 16, the subject 18, and the cassette device 22, including the guide lines 46, which are positioned as shown in FIGS. 1, 2, and 4 through 6, a camera image including the guide lines 46 therein can reliably be obtained.

As described above, the web camera 330 is integral with the radiation source device 16 as a result of being incorporated in the radiation source device 16 near a region where radiation 12 is output. Since the web camera 330 captures a camera image of the region to be imaged of the subject 18 and the cassette device 22, including the guide lines 46, which are positioned as shown in FIGS. 1, 2, 3B, and 4, a camera image including the guide lines 46 therein can reliably be obtained.

While operating the portable information terminal 34, the operator 32 instructs the subject 18 to position the subject 18 with respect to the guide lines 46. Even if radiation 12 is applied from the radiation source 14 to the subject 18 while the operator 32 operates the portable information terminal 34, the operator 32 is reliably prevented from being exposed to radiation 12.

The communication unit 218 of the portable information terminal 34, which incorporates the web camera 30, sends a camera image to the medical organization 40 via the antenna 216 and the network 36. Consequently, the camera image can reliably be sent to the medical organization 40.

The control processor 222 of the portable information terminal 34 generates a synchronization control signal, which synchronizes outputting of radiation 12 from the radiation source 14, and conversion of radiation 12 into a radiographic image in the radiation detector 20 with each other. The communication unit 218 sends the synchronization control signal to the communication unit 136 of the radiation source device 16 and to the communication unit 170 of the cassette device 22. Therefore, the radiation source 14 and the radiation detector 20 can reliably be synchronized during times that radiographic images are being captured.

Furthermore, inasmuch as the camera images captured by the web cameras 30, 330 and the radiographic image are sent from the portable information terminal 34 to the medical organization 40 by way of wireless communications via the network 36, the doctor 38 at the medical organization 40 can give appropriate instructions to the operator 32 and the subject 18 at the site by visually recognizing the camera images and the radiographic image, which are displayed on the display unit 112 of the console 106.

The console 106 includes the exposure switch 120 for initiating output of radiation 12 from the radiation source 14. If the doctor 38 turns on the exposure switch 120 based on camera images displayed on the display unit 112, the control processor 226 of the console 106 generates an exposure control signal for initiating output of radiation 12 from the radiation source 14, and sends the generated exposure control signal from the communication unit 104 to the portable information terminal 34 via the network 36. The control processor 222 of the portable information terminal 34 generates a synchronization control signal based on the exposure control signal received by the communication unit 218, and sends the generated synchronization control signal to the radiation source device 16 and the cassette device 22.

Consequently, the doctor 38 can capture a radiographic image of the subject 18 in real time while monitoring the subject 18 at the medical organization 40 where the doctor 38 is unable to observe the subject 18 directly, without requiring the doctor 38 to travel to the disaster site or the home care treatment site.

More specifically, in a case where image capturing preparations have been completed, if the region to be imaged of the subject 18 is included within the outer frame of the guide lines 46, as shown in the camera images captured by the web cameras 30, 330, the doctor 38 turns on the exposure switch 120 to start capturing a radiographic image of the subject 18. On the other hand, if the region to be imaged of the subject 18 is not included within the outer frame of the guide lines 46 in the camera images, or if only a portion of the region to be imaged is included within the outer frame of the guide lines 46 in the camera images, the doctor 38 does not turn on the exposure switch 120, but instead instructs the operator 32 to carry out image capturing preparations once again.

Thus, an image capturing process can easily and reliably be performed under remote control from the medical organization 40.

The doctor 38 instructs the operator 32 at the site through screens, which are displayed on the display unit 64, and via speech sounds, which are output from the speakers 78. Accordingly, the doctor 38 can accurately and efficiently send instructions to the operator 32 at the site.

If the camera images output from the web cameras 30, 330 are moving images, or still images that are captured intermittently at given time intervals, the doctor 38 can provide timely instructions to the operator 32 at the site. Even if the camera images are still images captured at certain times during image capturing preparations, the doctor 38 can judge whether or not the subject 18 is in a state that enables radiographic images to be captured by observing the still images.

If the web cameras 30, 330 are optical cameras, then the web cameras 30, 330 can produce camera images that are highly visible to the doctor 38.

It has been described above that the doctor 38 provides instructions to the operator 32 through screens that are displayed on the display unit 64, and via speech sounds that are output from the speakers 78. However, the doctor 38 may provide instructions to the operator 32 only through the screens displayed on the display unit 64, or only via speech sounds output from the speakers 78.

It also has been described above that the doctor 38 gives instructions to the operator 32 and the operator 32 positions the subject 18 according to the content of such instructions. Since speech sounds output from the speakers 78 are heard by the subject 18, the doctor 38 may send instructions directly to the subject 18 in order to position the subject 18. Alternatively, before the subject 18 is positioned, the subject 18 may confirm the content of the instructions, which are displayed on the display unit 64, and the subject 18 may position him or herself with respect to the guide lines according to the instructions.

It also has been described above that the portable information terminal 34 sends the synchronization control signal to the radiation source device 16 and the cassette device 22. Instead, the control signal generator 346 of the console 106 may generate a synchronization control signal, and may send the generated synchronization control signal to the radiation source device 16 and the cassette device 22 via the network 36 and the portable information terminal 34.

Rather than activating the cassette device 22 by turning on the switch 50, the operator 32 may activate the cassette device 22 by operating the operating unit 60. Alternatively, the doctor 38 may activate the cassette device 22 by operating the operating unit 114. Alternatively, the operator 32 may activate the cassette device 22 by gripping the handle 310 to cause the touch sensor 312 to output a detection signal.

The handle 310 that is gripped by the operator 32 is disposed on a side of the radiation source device 16 opposite from the side on which radiation 12 is output. The operator 32 can grip the handle 310 with one hand to orient the radiation source device 16 incorporating the web camera 330 therein toward the subject 18 and the cassette device 22, and at the same time, the operator 32 can operate the portable information terminal 34 with the other hand while observing the display unit 64. Since the camera image captured by the web camera 330 is displayed on the display unit 64, by observing the displayed camera image, the operator 32 can position the subject 18 while moving the radiation source device 16 to a desired position. Even if radiation 12 is emitted while the operator 32 is gripping the handle 310, radiation 12 is reliably prevented from being applied toward the operator 32, i.e., the operator 32 is reliably prevented from being exposed to radiation 12.

The present exemplary embodiments are applicable effectively to disaster sites where obstacles may be present. Since there are many obstacles, and it is difficult to move the subject 18 due to injury of the subject 18 at the disaster site, as a practical matter, it is difficult to secure the radiation source device 16 and the cassette device 22 at given positions and to guide the subject 18 to a position between the radiation source device 16 and the cassette device 22. Accordingly, the radiation source device 16 and the cassette device 22 frequently need to be located at positions that are convenient to the subject 18. Although the operator 32 can direct the radiation source device 16 toward the subject 18, it may be difficult to observe the subject 18 directly due to the presence of obstacles. Consequently, it may not be easy to position the subject 18.

According to the present exemplary embodiment, if the operator 32 grips the handle 310 with one hand and directs the radiation source device 16 toward the subject 18 and the cassette device 22, the web camera 330 captures a camera image of the subject 18 and the cassette device 22, and the captured camera image is displayed on the display unit 64. Thus, the operator 32 can easily operate the portable information terminal 34 with the other hand, or adjust the position of the radiation source device 16 and position the subject 18, while viewing the camera image displayed on the display unit 64.

Since the handle 310 includes an electrostatic capacitance or resistance film type of touch sensor 312, the radiation source controller 138 or the control processor 222 is able to activate the radiation source device 16 or operate the cassette device 22 based on the aforementioned detection signal.

The portable information terminal 34, the radiation source device 16, and the cassette device 22 may be electrically connected to each other by USB cables, not shown. Further, the battery 220 of the portable information terminal 34 may charge the battery 134 of the radiation source device 16 and the battery 166 of the cassette device 22. Such a charging configuration allows the batteries 134, 166 to be charged while also allowing signals to be sent and received reliably. More specifically, the portable information terminal 34 can reliably send synchronization control signals and image capturing conditions to the radiation source device 16 and the cassette device 22, and the cassette device 22 can reliably send radiographic images to the portable information terminal 34.

In a case where the batteries 134, 166 are charged, the batteries 134, 166 may be charged to a capacity that is large enough to capture at least as many radiographic images of the subject 18 as required. Accordingly, required radiographic images of the subject 18 can reliably be captured.

Alternatively, the batteries 134, 166 may be charged only during a time period before radiographic images are captured. Thus, since the batteries 134, 166 are not charged while radiographic images are being sent before and after radiographic images are captured, noise caused by battery charging is prevented from being added to the electric charge signals (analog signals), or from being added to the radiographic image while radiographic images are being sent.

It has been described above that a radiographic image begins to be captured if the exposure switch 120 is turned on, or if the icon 572 is clicked. Since a radiographic image may start to be captured by an instruction from the doctor 38, an exposure button (exposure switch) may be displayed on the screen of the display unit 112, which is a touch panel display unit, for example, and the doctor 38 may press the displayed exposure button to start capturing a radiographic image. Alternatively, one button on the operating unit 114 may be used as a dedicated exposure switch, whereby a radiographic image starts to be captured by pressing the button.

The cassette device 22 is in the shape of a box. However, a portion of the cassette device 22 where the radiation detector 20, etc., is positioned may be in the shape of a flexible sheet. Since the flexible sheet can be wound into a roll, the overall radiographic image capturing apparatus 10 including the cassette device 22 can be reduced in size and weight.

For charging the batteries 134, 166 with the battery 220, one of the three batteries may be regarded as a power supply for the overall radiographic image capturing apparatus 10, which is capable of charging the other remaining batteries.

It has been described above that the camera image captured by the web camera 30 is sent from the communication unit 218 of the portable information terminal 34 to the communication unit 104 of the medical organization 40 via the network 36. However, the present exemplary embodiments are not limited to such a configuration.

The communication unit 136 of the radiation source device 16 and the communication unit 170 of the cassette device 22 may include a function for communicating with the communication unit 104 via the network 36, whereby a camera image can be sent from the communication units 136, 170.

With the communicating function incorporated in the communication units 136, 170, it is possible for the communication unit 170 to send radiographic images directly to the communication unit 104 via the network 36 and also to send radiographic images to the communication unit 104 via the communication unit 136 and the network 36.

Furthermore, all of the signals may be sent and received between the radiographic image capturing apparatus 10 and the medical organization 40 through the communication units 136, 104 or through the communication units 170, 104.

According to the present exemplary embodiments, since the portable information terminal 34, the radiation source device 16, and the cassette device 22 are connected via the same link (communications link) by way of wireless communications, and a cable (USB cable) is not required to send and receive signals, the operator 32 does not encounter obstacles in carrying out work. Therefore, the operator 32 can work efficiently. Further, since cables are not required, the number of parts of the radiographic image capturing apparatus 10 is reduced, and the radiographic image capturing apparatus 10 can be assembled with ease at the site.

Since the portable information terminal 34, the radiation source device 16, and the cassette device 22 are provided in the same link as described above, signals representative of camera images, radiographic images, etc., may be sent to and received from the communication unit 104 of the medical organization 40 through any one of the communication units 136, 170, 218.

According to the present exemplary embodiments, signals may be sent and received by way of optical wireless communications using infrared rays, rather than wireless communications.

As described above, signals are sent and received between the radiographic image capturing apparatus 10 and the medical organization 40 via the network 36 by way of wireless communications. However, the present exemplary embodiments are not limited to such a configuration. Signals may be sent and received by way of other forms of communication.

More specifically, signals may be sent and received between the radiographic image capturing apparatus 10 and the medical organization 40 by way of wired communications via the network 36.

Alternatively, signals may be sent and received by way of wired and wireless communications via the network 36. More specifically, if the network 36 includes a repeater (repeating device), then signals may be sent and received by way of wired communications (or wireless communications) up to the repeater, and then sent and received by way of wireless communications (or wired communications) beyond the repeater.

Another portable terminal such as a mobile telephone or the like may be electrically connected to the portable information terminal 34. Signals may be sent and received between the portable information terminal 34 and the medical organization 40, or may be sent and received between the radiation source device 16 and the cassette device 22, using a communicating function of the other portable terminal. According to this modification, the communication unit of the other portable terminal functions as the communication unit 218.

The present exemplary embodiments are applicable to acquisition of radiographic images using a light readout type of radiation detector. Such a light readout type radiation detector operates in the following manner. If radiation is applied to a matrix of solid-state detecting devices, the solid-state detecting devices store an electrostatic latent image depending on the dose of radiation applied thereto. For reading the stored electrostatic latent image, reading light is applied to the solid-state detecting devices to cause the solid-state detecting devices to generate an electric current representing a radiation image. If erasing light is applied to the radiation detector, a radiographic image represented by the residual electrostatic latent image is erased from the radiation detector, which can be reused (see Japanese Laid-Open Patent Publication No. 2000-105297).

In order to prevent the radiographic image capturing apparatus 10 from being contaminated with blood and bacteria, the entire radiographic image capturing apparatus 10 may be of a water-resistant and hermetically sealed structure, and the radiographic image capturing apparatus 10 can be sterilized and cleaned as necessary so that it can be used repeatedly.

In the present exemplary embodiments, as shown in FIG. 99, a cradle 230 for charging the batteries 134, 166, 220 (see FIG. 12) is positioned at a desired location in the medical organization 40.

The cradle 230 is electrically connected to the portable information terminal 34 by a USB cable 234 having connectors 236, 238, is electrically connected to the radiation source device 16 by a USB cable 24 having connectors 58, 82, and is electrically connected to the cassette device 22 by a USB cable 26 having connectors 52, 86.

The cradle 230 may be capable not only of charging the batteries 134, 166, 220, but may also have a wireless or wired communication function to send and receive necessary information to and from the console 106 and the RIS of the medical organization 40. Information that is sent from the cradle 230 may include radiographic images, which are recorded in the radiographic image capturing apparatus 10 that is connected to the cradle 230.

The cradle 230 has a display unit 232 for displaying a charged state of the radiographic image capturing apparatus 10 connected to the cradle 230, together with other necessary information including radiographic images acquired from the radiographic image capturing apparatus 10.

A plurality of cradles 230 may be connected to a network, and charged states of radiographic image capturing apparatus 10, which are connected to the cradles 230, may be retrieved through the network, so that the user can confirm the locations of radiographic image capturing apparatus 10 that are charged sufficiently, based on the retrieved charged stages.

The radiographic image capturing apparatus 10 according to the present exemplary embodiments has been illustrated as being used to capture radiographic images at disaster sites and home care treatment sites. However, the radiographic image capturing apparatus 10 according to the present exemplary embodiments is not limited to capturing radiographic images at disaster sites and home care treatment sites. Alternatively, the radiographic image capturing apparatus 10 may be mounted on medical checkup cars for capturing radiographic images for use in medical checkups, or may be used to capture radiographic images of patients during a doctor's rounds in the medical organization 40. Furthermore, the radiographic image capturing apparatus 10 according to the present exemplary embodiments is not limited to being used for capturing radiographic images in the medical field, but may be applied to capturing radiographic images in various nondestructive tests, for example.

### [Modifications of the Present Exemplary Embodiments]

Modifications ranging from first through eleventh modifications of the above exemplary embodiments will be described below with reference to FIGS. 100 through 110B.

Structural components of the modifications, which are identical to those shown in FIGS. 1 through 99, are denoted by identical reference characters, and such features will not be described in detail below. In FIGS. 100 through 110B, the network 36 and the medical organization 40 are omitted from illustration.

### [First Modification]

As shown in FIG. 100, a radiographic image capturing apparatus 10A and a radiographic image capturing system 11A according to a first modification differ from the exemplary embodiments shown in FIGS. 1 through 99, in that signals are sent and received between the portable information terminal 34, the radiation source device 16, and the cassette device 22 by way of wired communications.

The portable information terminal 34, the radiation source device 16, and the cassette device 22 are electrically connected to each other by USB cables 24 and 26. Therefore, the battery 220 (see FIG. 12) of the portable information terminal 34 can reliably charge the battery 134 of the radiation source device 16 and the battery 166 of the cassette device 22, and also can reliably send and receive signals. Since electric power can be supplied and signals can be sent and received (transmitted) by way of wired communications using the USB cables 24, 26, a synchronization control signal and image capturing conditions can reliably be sent from the portable information terminal 34 to the radiation source device 16 and the cassette device 22. Also, radiographic images can reliably be sent from the cassette device 22 to the portable information terminal 34.

### [Second Modification]

As shown in FIG. 101, a radiographic image capturing apparatus 10B and a radiographic image capturing system 11B according to a second modification differ from the exemplary embodiments shown in FIGS. 1 through 100, in that the web camera 330 is included in the radiation source device 16, and the web camera 30 is not provided.

The web camera 330 captures an image of the face including the pupils of the subject 18 before a radiographic image is captured. In other words, the web camera 330 also carries out the functions of the web camera 30 according to the exemplary embodiments shown in FIGS. 1 through 100.

Since the second modification is the same as the exemplary embodiments shown in FIGS. 1 through 100 except that the web camera 30 is not provided, the second modification offers the same advantages as those of the exemplary embodiments shown in FIGS. 1 through 100, apart from advantages that come about due to the presence of the web camera 30.

### [Third Modification]

As shown in FIG. 102, a radiographic image capturing apparatus 10C and a radiographic image capturing system 11C according to a third modification differ from the exemplary embodiments shown in FIGS. 1 through 100, in that the web camera 30 is included in the radiation source device 16, and the web camera 330 is not provided.

Since the third modification is the same as the exemplary embodiments shown in FIGS. 1 through 100, except that the web camera 330 is not provided, the third modification offers the same advantages as those of the exemplary embodiments shown in FIGS. 1 through 100, apart from advantages that come about due to the presence of the web camera 330.

### [Fourth Modification]

As shown in FIG. 103, a radiographic image capturing apparatus 10D and a radiographic image capturing system 11D according to a fourth modification differs from the second modification (see FIG. 101), in that the web camera 330 and the radiation source device 16 are connected to each other (i.e., are made integral with each other) by a USB cable 240.

Since the fourth modification is the same as the second modification, except that the web camera 330 is connected to the radiation source device 16 by the USB cable 240, the fourth modification offers the same advantages as those of the second modification. Furthermore, inasmuch as the web camera 330 can be placed as a standalone camera in a desired position, within a range defined by the length of the USB cable 240, the web camera 330 can be positioned with greater freedom than if the web camera 330 were incorporated in the radiation source device 16. The web camera 330 includes a communication unit 260 for directly sending and receiving signals representative of radiographic images, camera images, etc., to and from the communication unit 104 of the medical organization 40 via the network 36 (see FIG. 1).

### [Fifth Modification]

As shown in FIG. 104, a radiographic image capturing apparatus 10E and a radiographic image capturing system 11E according to a fifth modification differ from the third modification (see FIG. 102), in that the web camera 30 and the portable information terminal 34 are connected to each other (i.e., are made integral with each other) by way of a USB cable 240.

Since the fifth modification is the same as the third modification, except that the web camera 30 is connected to the portable information terminal 34 by the USB cable 240, the fifth modification offers the same advantages as those of the third modification. Furthermore, inasmuch as the web camera 30 can be placed as a standalone camera in a desired position, within a range defined by the length of the USB cable 240, the web camera 30 can be positioned with greater freedom than if the web camera 30 were incorporated in the portable information terminal 34. The web camera 30 includes a communication unit 260 for directly sending and receiving signals representative of radiographic images, camera images, etc., to and from the communication unit 104 of the medical organization 40 via the network 36 (see FIG. 1).

### [Sixth Modification]

As shown in FIG. 105, a radiographic image capturing apparatus 10F and a radiographic image capturing system 11F according to a sixth modification differ from the exemplary embodiments shown in FIGS. 1 through 104, in that the web camera 30 (330), the portable information terminal 34, and the radiation source device 16 are separate from each other.

Since the portable information terminal 34, the radiation source device 16, and the cassette device 22 are wirelessly connected in a single link, signals representative of radiographic images, camera images, etc., may be sent to and received from the communication unit 104 (see FIGS. 1 and 13) of the medical organization 40 via any one of the communication units 136, 170, 218 (see FIG. 12). For example, the web camera 30 (330) may send a camera image from the communication unit 260 directly to the medical organization 40 via the network 36, or may send a camera image from the communication unit 260 indirectly to the medical organization 40 via the communication unit 136 of the radiation source device 16, the communication unit 170 of the cassette device 22, or the communication unit 218 of the portable information terminal 34. Thus, the doctor 38 is able to see the camera image captured by the web camera 30 (330).

Since the web camera 30 (330) is provided as a standalone camera, the web camera 30 (330) can be placed in any desired position, and hence can be positioned with increased freedom.

Since the sixth modification is essentially the same as the exemplary embodiments shown in FIGS. 1 through 104, except that signals are sent and received between the portable information terminal 34, the web camera 30 (330), the radiation source device 16, and the cassette device 22 by way of wireless communications, the sixth modification offers the same advantages as those of the exemplary embodiments shown in FIGS. 1 through 104, except that signals are sent and received by way of wireless communications.

### [Seventh Modification]

As shown in FIG. 106, a radiographic image capturing apparatus 10G and a radiographic image capturing system 11G according to a seventh modification differ from the exemplary embodiments shown in FIGS. 1 through 105, in that a web camera 246 (portable terminal camera, on-site camera) for capturing a camera image of the operator 32 in a case where the operator 32 operates the portable information terminal 34 is mounted on the lid 66.

The portable information terminal 34 sends a camera image (an image of the operator 32) captured by the web camera 246 to the medical organization 40 (see FIGS. 1 and 13) via the network 36. The operator 32 seeks an instruction concerning capturing of radiographic images while viewing the image of the doctor 38, which is displayed on the display unit 64. The doctor 38 issues an instruction to the operator 32 while viewing the image of the operator 32, which is displayed on the display unit 112. Since the operator 32 is familiar with the doctor 38 who is present in the remote medical organization 40, and the doctor 38 also is familiar with the operator 32 who is present at the site, the doctor 38 and the operator 32 can carry out image capturing preparations with confidence. The camera image captured by the web camera 246 may be associated with other information and stored in the memory 228, etc. Since the seventh modification is the same as the exemplary embodiments shown in FIGS. 1 through 105, except that the web camera 246 is added thereto, the seventh modification offers the same advantages as those of the exemplary embodiments shown in FIGS. 1 through 105.

### [Eighth Modification]

As shown in FIG. 107, a radiographic image capturing apparatus 10H and a radiographic image capturing system 11H according to an eighth modification differ from the exemplary embodiments shown in FIGS. 1 through 106, in that the radiation source device 16 is integral with the lid 66 as a result of being joined thereto.

The radiographic image capturing apparatus 10H can thus be assembled and accommodated more easily at the site. Since the radiation source device 16 and the portable information terminal 34 are made integral with each other, the battery 134, the communication unit 136, and the radiation source controller 138 (see FIG. 12) may be dispensed with. More specifically, the battery 220 is shared as a battery of the radiation source device 16, the control processor 222 is shared as a radiation source controller of the radiation source device 16, and the communication unit 218 is shared as a communication unit of the radiation source device 16. Thus, the radiation source device 16 is simplified in structure, thereby making the radiographic image capturing apparatus 10H smaller in size as a whole.

Since the radiation source device 16 and the portable information terminal 34 are made integral with each other, the operator 32 can change the position and direction of the portable information terminal 34 while viewing the display unit 64 or while operating the operating unit 60, thereby simultaneously adjusting the position and direction of the radiation source device 16 with respect to the cassette device 22 and the subject 18. According to the eighth modification, therefore, the position and direction of the radiation source device 16 with respect to the cassette device 22 and the subject 18 can be adjusted easily.

In FIG. 107, the web camera 30 (330) is incorporated in the lid 66. However, the web camera 30 (330) may be incorporated in the radiation source device 16. Since the eighth modification is the same as the exemplary embodiments shown in FIGS. 1 through 105, except for the fact that the radiation source device 16 and the portable information terminal 34 are made integral with each other, the eighth modification offers the same advantages as the exemplary embodiments shown in FIGS. 1 through 105, except that the radiation source device 16 and the portable information terminal 34 are made integral with each other.

### [Ninth Modification]

As shown in FIG. 108, a radiographic image capturing apparatus 10I and a radiographic image capturing system 11I according to a ninth modification differ from the exemplary embodiments shown in FIGS. 1 through 107, in that the radiation source 14 comprises a conventional thermionic-emission radiation source, and the radiographic image capturing apparatus 10I includes a high-voltage power supply 252 for energizing a filament of the radiation source 14.

The radiation source 14 and the communication unit 136 are housed in a casing 250, which is mounted on the upper end of a stand 248, and the casing 250 is electrically connected to the high-voltage power supply 252 by a USB cable 24. Further, the high-voltage power supply 252 and the portable information terminal 34 are electrically connected to each other by a USB cable 254 having connectors 256 and 258. The portable information terminal 34 can control the high-voltage power supply 252 to cause the radiation source 14 to emit radiation 12.

According to the ninth modification, the radiographic image capturing apparatus 10I is relatively large in size and has a relatively large number of parts, because the radiographic image capturing apparatus 10I includes a conventional thermionic-emission radiation source. However, the ninth modification offers the same advantages as those of the exemplary embodiments shown in FIGS. 1 through 107, except that the radiation source 14 is a thermionic-emission type.

As shown in FIG. 108, the high-voltage power supply 252 includes a communication unit 262, which is capable of sending and receiving signals representing radiographic images, camera images, etc., to and from the communication unit 104 of the medical organization 40 via the network 36 (see FIG. 1).

### [Tenth Modification]

As shown in FIG. 109, a radiographic image capturing apparatus 10J and a radiographic image capturing system 11J according to a tenth modification differ from the exemplary embodiments shown in FIGS. 1 through 108, in that the console 106 and a plurality of attaché cases 98, each of which houses therein the radiographic image capturing apparatus 10J, are provided in a medical checkup car 300 (waiting location) in which the doctor 38 is available, and the operator 32 (see FIG. 1) carries at least one of the attaché cases 98 from the medical checkup car 300 to the site. The cabin of the medical checkup car 300 where the doctor 38 is available serves as the waiting location from which the doctor 38 is unable to observe the subject 18 directly.

The radiographic image capturing apparatus 10J, which has been carried to the site, and the communication unit 104 in the medical checkup car 300 are capable of sending and receiving signals therebetween by way of wireless communications. For example, the radiographic image capturing apparatus 10J can send wireless signals representing camera images and radiographic images to the communication unit 104 by way of wireless communications. Therefore, the radiographic image capturing apparatus 10J offers the same advantages as the exemplary embodiments shown in FIGS. 1 through 108. Although the radiographic image capturing apparatus 10J and the communication unit 104 are illustrated in FIG. 109 as sending and receiving signals directly therebetween by way of wireless communications, the radiographic image capturing apparatus 10J and the communication unit 104 may send and receive signals therebetween by way of wireless communications via the network 36.

### [Eleventh Modification]

In each of the radiographic image capturing apparatus 10, 10A through 10J (radiographic image capturing systems 11, 11A through 11J), the radiation detector 20 may be constructed as shown in FIGS. 110A and 110B (eleventh modification). According to the eleventh modification, a specific structure of the radiation detector, which comprises a scintillator of CsI, will be described in detail below.

According to the eleventh modification shown in FIGS. 110A and 110B, the radiation detector 20 includes a scintillator 700 for converting radiation 12 that has passed through the subject 18 (see FIGS. 1, 2, 4 through 6, 12, and 100 through 109) into visible light (absorbing radiation 12 and emitting visible light), and a radiation detector 702 for converting visible light generated by the scintillator 700 into electric signals (electric charges) depending on a radiographic image. In FIGS. 110A and 111B, the grid 162 and the lead plate 164 (see FIGS. 4 through 6) are omitted from illustration.

As shown in FIGS. 110A and 110B, the radiation detector 20 may be a face-side readout type, i.e., and ISS (Irradiation Side Sampling) type, which includes the radiation detector 702 and the scintillator 700 arranged successively with respect to the irradiated surface 44 that is irradiated with radiation 12, or a reverse-readout type, i.e., a PSS (Penetration Side Sampling) type, which includes the scintillator 700 and the radiation detector 702 arranged successively with respect to the irradiated surface 44.

The scintillator 700 emits stronger light from the irradiated surface 44, which is irradiated with radiation 12. According to an ISS type, the light emission position of the scintillator 700 is closer to the radiation detector 702. Therefore, the ISS type allows the captured radiographic image to exhibit higher resolution, while also allowing the radiation detector 702 to detect a greater amount of visible light than a PSS type. The ISS type is thus effective to increase the sensitivity of the radiation detector 20 (cassette device 22) higher than that of a PSS type.

The scintillator 700 may be made of a material such as CsI:Tl (cesium iodide with added thallium), CsI:Na (sodium-activated cesium iodide), GOS (Gd₂O₂S:Tb), or the like.

FIG. 110B shows by way of example a scintillator 700, which includes a columnar crystalline region formed by evaporating a material containing CsI on an evaporated substrate 704.

More specifically, the scintillator 700 shown in FIG. 110B includes a columnar crystalline region of columnar crystals 700a near the irradiated surface 44 (the radiation detector 702) to which radiation 12 is applied, and a non-columnar crystalline region of non-columnar crystals 700b positioned remotely from the irradiated surface 44. The evaporated substrate 704 preferably is made of a highly heat resistant material, e.g., aluminum (Al), in light of its low cost. The columnar crystals 700a have an average diameter, which is substantially uniform along the longitudinal directions of the columnar crystals 700a.

As described above, the scintillator 700 is made up of the columnar crystalline region (columnar crystals 700a) and the non-columnar crystalline region (non-columnar crystals 700b), in which the columnar crystalline region, which comprises columnar crystals 700a that are capable of emitting light highly efficiently, is disposed near the radiation detector 702. Therefore, visible light generated by the scintillator 700 travels through the columnar crystals 700a and is emitted toward the radiation detector 702. As a result, visible light emitted toward the radiation detector 702 is prevented from scattering, which in turn prevents the radiographic image detected by the cassette device 22 from becoming blurred. Visible light that has reached a deeper region (non-columnar crystalline region) of the scintillator 700 is reflected toward the radiation detector 702 by the non-columnar crystals 700b. Consequently, the amount of visible light that is applied to the radiation detector 702 (the efficiency at which visible light emitted by the scintillator 700 is detected) can be increased.

If it is assumed that the thickness of the columnar crystalline region of the scintillator 700, which is positioned near the irradiated surface 44, is represented by t1, whereas the thickness of the non-columnar crystalline region of the scintillator 700, which is positioned near the evaporated substrate 704, is represented by t2, then the thicknesses t1, t2 preferably satisfy the relationship 0.01≦t2/t1≦0.25.

Since the thickness t1 of the columnar crystalline region and the thickness t2 of the non-columnar crystalline region satisfy the above relationship, the ratio along thickness-wise directions of the scintillator 700 between the region (columnar crystalline region), which has high light emission efficiency to prevent visible light from being scattered, and the region (non-columnar crystalline region), which reflects visible light, is in an appropriate range for increasing light emission efficiency of the scintillator 700, the efficiency at which the visible light emitted by the scintillator 700 is improved, together with enhancing the resolution of the radiographic image.

If the thickness t2 of the non-columnar crystalline region is too large, then a region with low light emission efficiency increases, resulting in a reduction in sensitivity of the cassette device 22. Therefore, the ratio (t2/t1) preferably is in a range from 0.02 to 0.1.

It has been described above that the scintillator 700 includes the columnar crystalline region and the non-columnar crystalline region in a continuous array. However, the scintillator 700 may include the columnar crystalline region and a light reflecting layer of Al or the like, which replaces the non-columnar crystalline region.

The radiation detector 702 serves to detect visible light emitted from the light emission side (columnar crystal 700a) of the scintillator 700. As shown in FIG. 110A, the radiation detector 702 includes an insulating substrate 708, a TFT layer 710, and photoelectric transducers 712, which are successively deposited on the irradiated surface 44 along the direction in which radiation 12 is applied. A planarization layer 714 is disposed in covering relation to the photoelectric transducers 712 on the bottom surface of the TFT layer 710.

The radiation detector 702 is constructed as a TFT active matrix board (hereinafter referred to as a “TFT board”) having a matrix of pixels 720 as viewed in plan on the insulating substrate 708. Each of the pixels 720 includes one of the photoelectric transducers 712, which may comprise a photodiode (PD) or the like, a storage capacitor 716, and a TFT 718.

The TFT 718 corresponds to the TFT 188 (see FIG. 11) described above, and the photoelectric transducer 712 and the storage capacitor 716 correspond to a pixel 180.

The photoelectric transducer 712 comprises a lower electrode 712a near the scintillator 700, an upper electrode 712b near the TFT layer 710, and a photoelectric conversion film 712c disposed between the lower electrode 712a and the upper electrode 712b. The photoelectric conversion film 712c absorbs visible light emitted from the scintillator 700, and generates electric charges depending on the absorbed visible light.

The lower electrode 712a preferably is made of an electrically conductive material, which is transparent at least to the wavelength of light emitted by the scintillator 700, because the lower electrode 712a needs to apply visible light emitted by the scintillator 700 to the photoelectric transducer 712. More specifically, it is preferable to make the lower electrode 712a of a transparent conducting oxide (TCO), which is highly permeable to visible light and has a low resistance value.

Although the lower electrode 712a may be made of a thin metal film of Au or the like, preferably, the lower electrode 712a is made of TCO, because the resistance value of a thin metal film of Au or the like tends to increase if the thin metal film has a light transmittance of 90% or higher. The lower electrode 712a may be made of ITO (Indium Tin Oxide), IZO (Indium Zinc Oxide), AZO (Aluminum-doped Zinc Oxide), FTO (Fluorine-doped Tin Oxide), SnO₂, TiO₂, ZnO₂, or the like. However, most preferably, the lower electrode 712a is made of ITO in light of processing simplicity, low resistance, and transparency. The lower electrode 712a may be a single electrode shared by all of the pixels 720, or may be divided into a plurality of electrodes assigned to each of the pixels 720 respectively.

The photoelectric conversion film 712c may be made of a material that absorbs visible light and generates electric charges. For example, the photoelectric conversion film 712c may be made of amorphous silicon (a-Si), or an organic photoelectric conversion material (OPC). If the photoelectric conversion film 712c is made of amorphous silicon, the photoelectric conversion film 712c can absorb visible light range emitted from the scintillator 700 in a wide wavelength range. However, since a photoelectric conversion film 712c made of amorphous silicon needs to be evaporated, heat resistance of the insulating substrate 708 should be taken into consideration if the insulating substrate 708 is made of synthetic resin.

If the photoelectric conversion film 712c is made of a material including an organic photoelectric conversion material, then the photoelectric conversion film 712c provides an absorption spectrum, which exhibits a high absorption rate mainly in the visible range, and hence does not absorb electromagnetic waves other than visible light emitted by the scintillator 700. Therefore, noise produced if radiation 12 such as X-rays, γ-rays, or the like is absorbed by the photoelectric conversion film 712c can be minimized.

The photoelectric conversion film 712c, which is made of an organic photoelectric conversion material, can be formed by applying an organic photoelectric conversion material to a target using a liquid droplet ejection head such as an ink jet head or the like. Therefore, the target does not need to be heat resistant. According to the eleventh modification, the photoelectric conversion film 712c is made of an organic photoelectric conversion material.

The photoelectric conversion film 712c, which is made of an organic photoelectric conversion material, absorbs almost no radiation 12. Therefore, in an ISS type of radiation detector 20 having the radiation detector 702 arranged to pass radiation 12 therethrough, attenuation of radiation 12 that has passed through the radiation detector can be reduced, thereby minimizing a reduction in sensitivity to radiation 12. Therefore, it is preferable for the photoelectric conversion film 712c to be made of an organic photoelectric conversion material, particularly if the radiation detector 20 is of an ISS type.

In order for the organic photoelectric conversion material of the photoelectric conversion film 712c to absorb visible light emitted by the scintillator 700 most efficiently, the absorption peak wavelength thereof should preferably be as close as possible to the light emission peak wavelength of the scintillator 700. Although the absorption peak wavelength of the organic photoelectric conversion material and the light emission peak wavelength of the scintillator 700 ideally are in agreement with each other, it is possible to sufficiently absorb light emitted by the scintillator 700 if the difference between the absorption peak wavelength and the light emission peak wavelength is small enough. More specifically, the difference between the absorption peak wavelength of the organic photoelectric conversion material and the light emission peak wavelength of the scintillator 700 with respect to the radiation 12 preferably is 10 nm or smaller, and more preferably, is 5 nm or smaller.

Organic photoelectric conversion materials that meet the above requirements include quinacridone-based organic compounds and phthalocyanine-based organic compounds, for example. Since quinacridone, for example, has an absorption peak wavelength of 560 nm in the visible range, if quinacridone is used as the organic photoelectric conversion material and CsI:Tl is used as the material of the scintillator 700, the difference between the above peak wavelengths can be reduced to 5 nm or smaller, thus making it possible to substantially maximize the quantity of electric charges generated by the photoelectric conversion film 712c.

The photoelectric conversion film 712c, which is applicable to the radiation detector 20, will be described in specific detail below.

The radiation detector 20 includes an electromagnetic wave absorption/photoelectric conversion region provided by an organic layer, including the upper electrode 712b and the lower electrode 712a, and the photoelectric conversion film 712c sandwiched between the upper electrode 712b and the lower electrode 712a. The organic layer may be formed by the superposition or mixture of an electromagnetic wave absorption region, a photoelectric conversion region, an electron transport region, a hole transport region, an electron blocking region, a hole blocking region, a crystallization prevention region, an electrode, and an interlayer contact improving region, etc.

The organic layer preferably includes an organic p-type compound or an organic n-type compound. An organic p-type semiconductor (compound) is a donor organic compound typified mainly by a hole transport organic compound, and refers to an organic compound that tends to donate electrons. More specifically, in a case where two organic materials are placed in contact with each other, one of the organic materials, which has a lower ionization potential, is referred to as a donor organic compound. Any type of organic compound which is capable of donating electrons can be used as a donor organic compound. An organic n-type semiconductor (compound) is an acceptor organic compound typified mainly by an electron transport organic compound, and refers to an organic compound that tends to accept electrons. More specifically, in a case where two organic materials are placed in contact with each other, one of the organic materials, which has a larger electron affinity, is referred to as an acceptor organic compound. Any type of organic compound which is capable of accepting electrons can be used as an acceptor organic compound.

Materials that can be used as the organic p-type semiconductor and the organic n-type semiconductor, and arrangements of the photoelectric conversion film 712c are disclosed in detail in Japanese Laid-Open Patent Publication No. 2009-032854, and will not be described in detail below.

The photoelectric transducer 712 of each pixel may include at least the upper electrode 712b, the lower electrode 712a, and the photoelectric conversion film 712c. For preventing dark current from increasing, the photoelectric transducer 712 preferably includes at least one of an electron blocking film and a hole blocking film, and more preferably, includes both the electron blocking film and the hole blocking film.

The electron blocking film may be disposed between the upper electrode 712b and the photoelectric conversion film 712c. If a bias voltage is applied between the upper electrode 712b and the lower electrode 712a, the electron blocking film can prevent electrons from being injected from the upper electrode 712b into the photoelectric conversion film 712c, thereby preventing dark current from increasing. The electron blocking film may be made of an organic material that can donate electrons. The electron blocking film actually is made of a material which is selected depending on the material of the adjacent electrode and the material of the adjacent photoelectric conversion film 712c. A preferable material has an electron affinity (Ea), which is at least 1.3 eV greater than the work function (Wf) of the material of the adjacent electrode, and an ionization potential (Ip) equal to or smaller than the Ip of the material of the adjacent photoelectric conversion film 712c. Materials that can be used as an organic material and which are capable of donating electrons are disclosed in detail in Japanese Laid-Open Patent Publication No. 2009-032854, and such materials will not be described in detail below.

The thickness of the electron blocking film preferably is in the range from 10 nm to 200 nm, more preferably, is in the range from 30 nm to 150 nm, and particularly preferably, is in the range from 50 nm to 100 nm, in order to reliably achieve a dark current reducing capability and to prevent the photoelectric conversion efficiency of the photoelectric transducer 712 from being lowered.

The hole blocking film may be disposed between the photoelectric conversion film 712c and the lower electrode 712a. In the case that a bias voltage is applied between the upper electrode 712b and the lower electrode 712a, the hole blocking film can prevent holes from being injected from the lower electrode 712a into the photoelectric conversion film 712c, thereby preventing dark current from increasing. The hole blocking film may be made of an organic material capable of accepting electrons. The hole blocking film actually is made of a material, which is selected depending on the material of the adjacent electrode and the material of the adjacent photoelectric conversion film 712c. A preferable material should have an ionization potential (Ip), which is at least 1.3 eV greater than the work function (Wf) of the material of the adjacent electrode, and an electron affinity (Ea), which is equal to or greater than the Ea of the material of the adjacent photoelectric conversion film 712c. Materials that can be used as an organic material and which can accept electrons are disclosed in detail in Japanese Laid-Open Patent Publication No. 2009-032854, and such materials will not be described in detail below.

The thickness of the hole blocking film preferably is in the range from 10 nm to 200 nm, more preferably, is in the range from 30 nm to 150 nm, and particularly preferably, is in the range from 50 nm to 100 nm, in order to reliably achieve a dark current reducing capability and to prevent the photoelectric conversion efficiency of the photoelectric transducer 712 from being lowered.

For setting a bias voltage to move holes, which exist among the electric charges generated in the photoelectric conversion film 712c, toward the lower electrode 712a, and to move electrons, which exist among the electric charges generated in the photoelectric conversion film 712c, toward the upper electrode 712b, the electron blocking film and the hole blocking layer may be switched in position. Both the electron blocking film and the hole blocking layer are not necessarily required, and either one of them may be included to provide a certain dark current reducing capability.

Each of the TFTs 718 of the TFT layer 710 includes a stacked assembly of a gate electrode, a gate insulating film, and an active layer (channel layer). A source electrode and a drain electrode are disposed on the active layer and are spaced from each other with a gap therebetween. The active layer may be made of any one of amorphous silicon, an amorphous oxide, an organic semiconductor material, and carbon nanotubes. However, the active layer is not limited to being made from such materials.

Amorphous oxide, which the active layer may be made of, preferably is an oxide (e.g., In—O oxide) including at least one of In, Ga, and Zn, more preferably, is an oxide (e.g., In—Zn—O oxide, In—Ga oxide, or Ga—Zn—O oxide) including at least two of In, Ga, and Zn, and particularly preferably, is an oxide including In, Ga, and Zn. An In—Ga-An-O amorphous oxide preferably is an amorphous oxide the crystalline composition of which is represented by InGaO₃ (ZnO)ₘ where m represents a natural number smaller than 6, and particularly preferably, is InGaZnO₄. The amorphous oxide, which the active layer may be made of, is not limited to these materials.

The organic semiconductor material, which the active layer may be made of, preferably is a phthalocyanine compound, pentacene, vanadyl phthalocyanine, or the like, but is not limited to these materials. The composition of such a phthalocyanine compound is disclosed in detail in Japanese Laid-Open Patent Publication No. 2009-212389, and will not be described in detail below.

If the active layer of each of the TFTs 718 is made of one of an amorphous oxide, an organic semiconductor material, and carbon nanotubes, then the active layer is effective at reducing noise generated in the radiation detector 702, because the active layer does not absorb radiation 12 such as X-rays, or absorbs only an extremely small amount of radiation 12.

If the active layer is made of carbon nanotubes, then the TFTs 718 can have a high switching speed and exhibit a low absorption rate for visible light in the TFTs 718. If the active layer is made of carbon nanotubes, since the performance of the TFTs 718 could be greatly degraded by trace metal impurities mixed therewith, it is necessary to separate and extract highly pure carbon nanotubes using a centrifugal separator or the like.

Films made of an organic photoelectric conversion material, and films made of an organic semiconductor material are sufficiently flexible. If the photoelectric conversion film 712c, which is made of an organic photoelectric conversion material, and the TFT 718, the active layer of which is made of an organic semiconductor, are combined, then the radiation detector 702, on which the weight of the subject 18 is applied as a load, does not necessarily need to be made highly rigid.

The insulating substrate 708 may be made of any material insofar as the material is permeable to light and does not absorb a significant amount of radiation 12. The amorphous oxide of the active layer of the TFT 718 and the organic photoelectric conversion material of the photoelectric conversion film 712c of the photoelectric transducer 712 can be deposited as films at low temperatures. Therefore, the insulating substrate 708 is not limited to a highly heat-resistant substrate, such as a semiconductor substrate, a quartz substrate, a glass substrate, or the like, but may be a flexible substrate made of synthetic resin, a substrate of aramid fibers, or a substrate of bionanofibers. More specifically, the insulating substrate 708 may be a flexible substrate made of polyester such as polyethylene terephthalate, polybutylene phthalate, polyethylene naphthalate, or the like, polystyrene, polycarbonate, polyethersulfone, polyarylate, polyimide, polycycloolefine, norbornene resin, poly(chlorotrifluoroethylene), or the like. The flexible substrate made of plastic enables the radiation detector 20 to be light in weight and hence easy to carry around. The insulating substrate 708 may include an insulating layer for making the insulating substrate 708 electrically insulative, a gas barrier layer for making the insulating substrate 708 impermeable to water and oxygen, and an undercoat layer for making the insulating substrate 708 flat or to facilitate intimate contact with the electrode.

Since a high-temperature process at 200 degrees Celsius may be applied to aramid fibers, aramid fibers allow a transparent electrode material to be set at a high temperature for providing lower resistance. Such aramid fibers also allow driver ICs to be automatically mounted thereon by a process including a solder reflow process. Furthermore, inasmuch as aramid fibers have a coefficient of thermal expansion close to ITO and glass, an insulating substrate made of aramid fibers is less liable to warp and crack after fabrication. In addition, an insulating substrate made of aramid fibers may be made thinner than a glass substrate or the like. Such an insulating substrate 708 may be in the form of a stacked assembly made of an ultrathin glass substrate together with aramid fibers.

Bionanofibers are made by compounding a bundle of cellulose microfibrils (bacteria cellulose) produced by bacteria (acetic acid bacteria, Acetobacter Xylinum) and a transparent resin. The bundle of cellulose microfibrils has a width of 50 nm, which is 1/10 of the wavelength of visible light, is highly strong and highly resilient, and is subject to low thermal expansion. Bionanofibers that contain 60% to 70% fibers and which exhibit a light transmittance of about 90% at a wavelength of 500 nm can be produced by impregnating bacteria cellulose with a transparent resin such as an acrylic resin, an epoxy resin, or the like, and setting the transparent resin. In addition to being flexible, bionanofibers have a low coefficient of thermal expansion ranging from 3 ppm to 7 ppm, which is comparable to silicon crystals, a high strength of 460 MPa that matches the strength of steel, and a high resiliency of 30 GPa. Therefore, an insulating substrate 708 made of bionanofibers can be thinner than glass substrates or the like.

If a glass substrate is used as the insulating substrate 708, then the radiation detector 702 (TFT substrate) has an overall thickness of about 0.7 mm, for example. According to the eleventh modification, the insulating substrate 708 comprises a thin substrate made of a light-permeable synthetic resin, in order to make the cassette device 22 thinner. The overall thickness of the insulating substrate 708 is reduced to about 0.1 mm, thereby making the radiation detector 702 flexible. The radiation detector 702, which is made flexible in this manner, increases the shock resistance of the cassette device 22, such that the radiation detector 702 is less liable to break even if the cassette device 22 is subjected to shocks. Plastic resins, aramid fibers, bionanofibers, etc., do not absorb radiation 12 significantly. Thus, if the insulating substrate 708 is made of any of the aforementioned materials, the amount of radiation 12 absorbed by the insulating substrate 708 can be reduced. Consequently, even though radiation 12 passes through an ISS type of radiation detector 702, a reduction in sensitivity to radiation 12 is minimized.

The insulating substrate 708 of the cassette device 22 need not necessarily comprise a substrate of synthetic resin, but may be a substrate made of another material, such as a glass substrate or the like.

A planarization layer 714 for planarizing the radiation detector 702 is disposed on a side of the radiation detector 702 (TFT substrate), which is remote from the side (near the scintillator 700) to which radiation 12 is applied.

According to the eleventh modification, the radiation detector 20 may be constructed in the following manner.

(1) The photoelectric transducer 712 including the PD may be made of an organic photoelectric conduction material, and the TFT layer 701 may comprise CMOS sensors. Since only the PD is made of an organic material, the TFT layer 710 including the CMOS sensors is not required to be flexible. The photoelectric transducer 712, which is made of an organic photoelectric conduction material, and the CMOS sensors are disclosed in Japanese Laid-Open Patent Publication No. 2009-212377, and will not be described in detail below.

(2) The photoelectric transducer 712 including the PD may be made of an organic photoelectric conduction material, and the TFT layer 701, which is flexible, may comprise a CMOS circuit having TFTs made of an organic material. Pentacene may be used as a p-type organic semiconductor material used in the CMOS circuit, and cupric fluoride phthalocyanine (F₁₆CuPc) may be used as an n-type organic semiconductor material. Thus, the TFT layer 710 is made flexible enough to achieve a smaller radius of curvature. With the TFT layer 710 being constructed in this manner, the gate insulating film may be made significantly thinner for thereby reducing the drive voltage. The gate insulating film, the semiconductor, and the electrodes may be fabricated at room temperature or at a temperature of 100° C. or lower. The CMOS circuit may be fabricated directly on the flexible insulating substrate 708. The TFTs, which are made of an organic material, can be microfabricated by a fabrication process according to a scaling law. The insulating substrate 708 can be formed as a flat substrate that is free of surface irregularities by coating a thin polyimide substrate with a polyimide precursor according to a spin-coating process, and heating the coated substrate to change the polyimide precursor into polyimide.

(3) The PDs and TFTs, which are made of crystalline Si, may be disposed on a resin insulating substrate 708 according to a fluidic self-assembly process for placing a plurality of micron order device blocks in given positions on a substrate. More specifically, the PDs and TFTs as minute device blocks on a micron order are fabricated on another substrate, and then are cut off from the substrate. Then, the PDs and TFTs are scattered over the insulating substrate 708 as a target substrate and placed statistically thereon. The insulating substrate 708 is processed to match the device blocks, so that the device blocks can selectively be placed on the insulating substrate 708. Therefore, optimum device blocks (PDs and TFTs), which are made of an optimum material, can be integrated on an optimum substrate (insulating substrate 708). Therefore, it is possible to integrate PDs and TFTs on a non-crystalline insulating substrate 708 (resin substrate).

### [Other Arrangements of Present Exemplary Embodiment]

According to the present exemplary embodiment, as shown in FIGS. 111A and 111B, the radiation source device 16 includes a recess 324 defined in a side remote from the side where radiation 12 is emitted, and a collapsible handle 320 disposed in the recess 324. A touch sensor 322, which has the same function as the touch sensor 312, is incorporated in the handle 320.

In a case where the operator 32 is not carrying the radiation source device 16, the handle 320 is accommodated inside the recess 324, as shown in FIG. 111A. If the operator 32 turns the handle 320 about a proximal end, the handle 320 is raised out of the recess 324, so that the operator 32 can grip the handle 320. The handle 320 and the touch sensor 322 offer the same advantages as those of the handle 320 and the touch sensor 312 described above.

In a case where the handle 320 is accommodated in the recess 324 (i.e., in a case where the radiation source device 16 is moved as shown in FIG. 7), the electrodes of the touch sensor 312 are kept out of contact with the hand of the operator 32. Therefore, during activation of the radiation source device 16, the radiation source 14 is prevented from emitting radiation 12 in error.

In the above exemplary embodiment, signals are sent and received by way of at least one of wireless communications and wired communications. However, if the subject 18 is held in contact with the radiation source device 16 and the cassette device 22 with a short SID, then signals (e.g., a synchronization control signal) may be sent and received between the radiation source device 16 and the cassette device 22 by way of intrabody communications through the subject 18. Further, if the operator 32 is held in contact with both the radiation source device 16 and the portable information terminal 34, then signals may be sent and received between the radiation source device 16 and the portable information terminal 34 by way of intrabody communications through the subject 18.

The present invention is not limited to the above exemplary embodiments, and various alternative arrangements may be adopted without departing from the scope of the invention.

1. A radiographic image capturing system comprising:
a radiation source for outputting radiation;
a radiation detector for detecting radiation that has passed through a subject and converting the detected radiation into a radiographic image upon application of the radiation to the subject from the radiation source;
a portable terminal for controlling the radiation source and the radiation detector based on an image capturing menu concerning capturing of the radiographic image;
an on-site camera disposed together with the radiation source, the radiation detector, and the portable terminal at a site where the subject is present, for capturing an on-site image representative of the site;
an on-site communication unit disposed at the site;
a console disposed at a waiting location where a doctor or radiological technician having authority to apply the radiation to the subject waits while being unable to see the subject directly; and
a waiting location communication unit disposed at the waiting location,
wherein the on-site communication unit and the waiting location communication unit are able to send signals to and receive signals from each other; and
the waiting location communication unit transmits at least the image capturing menu to the on-site communication unit.

2. The radiographic image capturing system according to claim 1, wherein the portable terminal includes a portable terminal display unit;
the console includes a console display unit;
the on-site communication unit transmits at least one of the radiographic image and the on-site optical image captured by the on-site camera to the waiting location communication unit; and
each of the portable terminal display unit and the console display unit is capable of displaying at least one of the on-site optical image, the radiographic image, and the image capturing menu, and of changing displayed content depending on how the radiographic image is captured.

3. The radiographic image capturing system according to claim 2, further comprising:
a waiting location camera for capturing a waiting location optical image representative of the waiting location,
wherein the waiting location communication unit transmits the waiting location optical image captured by the waiting location camera to the on-site communication unit; and
the portable terminal display unit is capable of displaying at least one of the waiting location optical image, the on-site optical image, the radiographic image, and the image capturing menu.

4. The radiographic image capturing system according to claim 3, wherein the on-site camera comprises a radiation source camera integral with the radiation source, a portable terminal camera integral with the portable terminal, or a camera separate from the radiation source and the portable terminal.

5. The radiographic image capturing system according to claim 4, further comprising:
a radiation source device accommodating the radiation source therein, the radiation source device being permeable to the radiation,
wherein the radiation source camera is made integral with the radiation source device by being accommodated in the radiation source device, or by being connected to the radiation source device through a cable; and
the portable terminal camera is made integral with the portable terminal by being accommodated in the portable terminal, or by being connected to the portable terminal through a cable.

6. The radiographic image capturing system according to claim 5, wherein the on-site communication unit transmits a radiation source optical image captured by the radiation source camera and a portable terminal optical image captured by the portable terminal camera to the waiting location communication unit;
the portable terminal display unit is capable of displaying at least one of the radiation source optical image, the portable terminal optical image, the waiting location optical image, the radiographic image, and the image capturing menu; and
the console display unit is capable of displaying at least one of the radiation source optical image, the portable terminal optical image, the radiographic image, and the image capturing menu.

7. The radiographic image capturing system according to claim 6, further comprising:
a cassette device accommodating the radiation detector, the cassette device being permeable to the radiation,
wherein the radiation source camera captures at least one of an image of the cassette device and an image of the subject;
the portable terminal camera captures at least one of an image of the cassette device, an image of the radiation source device, and an image of the subject; and
the waiting location camera captures at least an image of the doctor or radiological technician.

8. The radiographic image capturing system according to claim 1, wherein the on-site communication unit transmits at least the on-site optical image captured by the on-site camera to the waiting location communication unit; and
the console is capable of changing content of the image capturing menu depending on a situation of the subject included in the on-site optical image before the radiographic image is captured.

9. The radiographic image capturing system according to claim 8, wherein the portable terminal includes a judgment processor for judging whether or not the subject included in the on-site optical image and the subject according to the image capturing menu agree with each other; and
the judgment processor transmits a judgment result through the on-site communication unit to the waiting location communication unit.

10. The radiographic image capturing system according to claim 8, wherein the image capturing menu includes order information for requesting capturing of the radiographic image of the subject, a procedure representative of a region to be imaged of the subject and a direction along which the radiation is applied based on the order information, and image capturing conditions for applying the radiation to the subject; and
the console changes at least one of the order information, the procedure, and the image capturing conditions depending on a situation of the subject included in the on-site optical image.

11. The radiographic image capturing system according to claim 8, wherein the portable terminal includes a portable terminal display unit;
the console includes a console display unit;
the on-site communication unit transmits at least one of the on-site optical image and the radiographic image to the waiting location communication unit;
each of the portable terminal display unit and the console display unit is capable of displaying at least one of the on-site optical image, the radiographic image, and the image capturing menu; and
the console displays the on-site optical image and the image capturing menu together on the console display unit such that the on-site optical image is displayed at a greater scale than other images if the image capturing menu is changed.

12. The radiographic image capturing system according to claim 1, further comprising:
a memory,
wherein the on-site communication unit transmits at least one of the radiographic image and the on-site optical image captured by the on-site camera to the waiting location communication unit; and
the console or the portable terminal stores at least one of the image capturing menu, the radiographic image, and the on-site optical image in association with each other in the memory.

13. The radiographic image capturing system according to claim 12, further comprising:
a radiation source device accommodating the radiation source therein, the radiation source device being permeable to the radiation,
wherein the on-site camera comprises a radiation source camera integral with the radiation source, a portable terminal camera integral with the portable terminal, or a camera separate from the radiation source and the portable terminal;
the radiation source camera is made integral with the radiation source device by being accommodated in the radiation source device, or by being connected to the radiation source device through a cable;
the portable terminal camera is made integral with the portable terminal by being accommodated in the portable terminal, or by being connected to the portable terminal by a cable;
the on-site communication unit transmits a radiation source optical image captured by the radiation source camera and a portable terminal optical image captured by the portable terminal camera to the waiting location communication unit; and
the console or the portable terminal stores the image capturing menu, the radiographic image, the radiation source optical image, and the portable terminal optical image in association with each other in the memory.

14. The radiographic image capturing system according to claim 13, further comprising:
a cassette device accommodating the radiation detector, the cassette device being permeable to the radiation;
wherein the radiation source camera captures images of the cassette device and a region to be imaged of the subject who is positioned with respect to the cassette device;
the portable terminal camera captures an image of the subject, or an image of the cassette device, an image of a region to be imaged of the subject who is positioned with respect to the cassette device, and an image of the radiation source device; and
the console includes an authentication processor for judging whether or not the subject included in the portable terminal optical image and the subject according to the image capturing menu agree with each other.

15. The radiographic image capturing system according to claim 12, further comprising:
a waiting location camera for capturing a waiting location optical image representative of the waiting location,
wherein the waiting location communication unit transmits the waiting location optical image captured by the waiting location camera to the on-site communication unit; and
the console or the portable terminal stores the image capturing menu, the radiographic image, and the on-site optical image, or the image capturing menu, the radiographic image, the on-site optical image, and the waiting location optical image in association with each other in the memory.

16. An image displaying method comprising:
placing a radiation source, a radiation detector, a portable terminal, an on-site camera, and an on-site communication unit at a site where a subject is present, and placing a console and a waiting location communication unit at a waiting location where a doctor or radiological technician having authority to apply the radiation to the subject waits while being unable to see the subject directly;
capturing an on-site optical image representative of the site with the on-site camera, transmitting the on-site optical image from the on-site communication unit to the waiting location communication unit, and transmitting at least an image capturing menu from the waiting location communication unit to the on-site communication unit, for thereby allowing a portable terminal display unit of the portable terminal and a console display unit of the console to display at least one of the on-site optical image and the image capturing menu; and
controlling the radiation source and the radiation detector with the portable terminal based on the image capturing menu, so as to apply the radiation from the radiation source to the subject, and to convert the radiation that has passed through the subject into a radiographic image with the radiation detector, and transmitting the radiographic image from the on-site communication unit to the waiting location communication unit, for thereby allowing each of the portable terminal display unit and the console display unit to display at least one of the radiographic image, the on-site optical image, and the image capturing menu.

17. A radiographic image capturing method comprising:
placing a radiation source, a radiation detector, a portable terminal, an on-site camera, and an on-site communication unit at a site where a subject is present, and placing a console and a waiting location communication unit at a waiting location where a doctor or radiological technician having authority to apply the radiation to the subject waits while being unable to see the subject directly;
capturing an on-site optical image representative of the site with the on-site camera, and transmitting the on-site optical image from the on-site communication unit to the waiting location communication unit;
changing content of an image capturing menu with the console depending on a situation of the subject included in the on-site optical image;
transmitting at least the image capturing menu from the waiting location communication unit to the on-site communication unit; and
controlling the radiation source and the radiation detector with the portable terminal based on the image capturing menu received through the on-site communication unit, so as to apply the radiation from the radiation source to the subject, and to detect the radiation that has passed through the subject and convert the detected radiation into a radiographic image with the radiation detector.

18. A radiographic image capturing method comprising:
placing a radiation source, a radiation detector, a portable terminal, an on-site camera, and an on-site communication unit at a site where a subject is present, and placing a console and a waiting location communication unit at a waiting location where a doctor or radiological technician having authority to apply the radiation to the subject waits while being unable to see the subject directly;
capturing an on-site optical image representative of the site with the on-site camera, transmitting the on-site optical image from the on-site communication unit to the waiting location communication unit, and transmitting at least an image capturing menu from the waiting location communication unit to the on-site communication unit;
controlling the radiation source and the radiation detector with the portable terminal based on the image capturing menu, so as to apply the radiation from the radiation source to the subject, and to convert the radiation that has passed through the subject into a radiographic image with the radiation detector, and transmitting the radiographic image from the on-site communication unit to the waiting location communication unit; and
storing at least the image capturing menu, the radiographic image, and the on-site optical image in association with each other in a memory, with the console or the portable terminal.
